(19) United States
(12) Patent Application Publication
(10) Pub. No.: US 2014/0051729 A1
(43) Pub. Date: Feb. 20, 2014

(54) COMPOSITIONS AND METHODS FOR TREATING DEGENERATIVE MUSCLE CONDITIONS

(71) Applicant: University of Washington, Seattle, WA (US)

(72) Inventors: Nicholas Ieronimakis, Seattle, WA (US); Hannele Ruohola-Baker, Seattle, WA (US); Morayma Reyes, Seattle, WA (US); Junlin Qi, Seattle, WA (US); Mario Pantoja, Seattle, WA (US); Karin Fischer, Seattle, WA (US)

(73) Assignee: University of Washington, Seattle, WA (US)

(21) Appl. No.: 13/941,355

(22) Filed: Jul. 12, 2013

Related U.S. Application Data
(63) Continuation-in-part of application No. PCT/US2012/021362, filed on Jan. 13, 2012.
(60) Provisional application No. 61/433,117, filed on Jan. 14, 2011.
(60) Provisional application No. 61/548,581, filed on Oct. 18, 2011.
(60) Provisional application No. 61/671,245, filed on Jul. 13, 2012.

(51) Int. Cl. C07D 417/04; C07D 233/64; C07D 403/04; C07D 401/04; A61K 31/4164; C07D 413/04

(52) U.S. Cl. CPC C07D 417/04; A61K 31/4164; C07D 413/04; C07D 403/04; C07D 401/04; C07D 233/64
USPC 514/341; 514/400; 514/378; 514/365; 514/383; 548/205; 548/247; 548/266.2; 546/275.1

(57) ABSTRACT

Methods and compositions for treating degenerative muscle conditions are disclosed. The compositions disclosed herein include S1P promoting compositions that include an S1P promoting agent. Embodiments of the methods disclosed herein include administering therapeutically effective amounts to subjects suffering from a degenerative muscle condition, such as, for example, subjects suffering from sarcopenia and subjects suffering from muscular dystrophy, including subjects suffering from Duchenne Muscular Dystrophy and Becker Muscular Dystrophy.

### RELATED APPLICATIONS

This application is a continuation-in-part of International Application No. PCT/US2012/021362, with an international filing date of Jan. 13, 2012, which application claims the benefit of U.S. Provisional Application No. 61/548,581, filed on Oct. 18, 2011, and U.S. Provisional Application No. 61/433,117, filed on Jan. 14, 2011. This continuation-in-part application claims the benefit of U.S. Provisional Application No. 61/671,245, filed Jul. 13, 2012. The entirety of each of these international and provisional applications is incorporated herein by reference.

### FEDERALLY SPONSORED RESEARCH OR DEVELOPMENT

This invention was made with government support under grant numbers 1RC1AR058520-01 and T32 AG00057 awarded by the National Institutes of Health. The government has certain rights in the invention.

### TECHNICAL FIELD

This disclosure relates to compositions and methods for the treatment of degenerative muscle conditions. Embodiments of the compositions and methods described herein are suited to inhibiting muscle degeneration associated with, for example, muscular dystrophy, and to regeneration of muscle in subjects suffering from a degenerative muscle condition.

### BACKGROUND

Muscular dystrophy refers to a group of hereditary muscle disorders characterized by progressive skeletal muscle weakness, defects in muscle proteins, and the death of muscle cells and tissue. Generally muscular dystrophies are multi-system disorders with manifestations in several different systems of the body ranging from the musculoskeletal system to the cardio-vascular, gastrointestinal, and nervous systems. The prognosis for people with muscular dystrophy varies according to the type and progression of the disorder. In some cases, the effects may be mild and progress slowly over a normal lifespan. In other cases, the disorder causes severe muscle weakness, functional disability, loss of the ability to walk, and premature death. There is no known cure or specific treatment for any of the muscular dystrophies.

Duchenne Muscular Dystrophy (DMD) is a severe, X-linked, recessive form of muscular dystrophy that affects 1 in 3500 male births and involves primary mutations in the gene encoding the muscle protein dystrophin. The dystrophin mutations in DMD patients result in dystrophin deficiencies and cause sarcolemmal instability, which leads to frequent muscle fiber damage and repair. In dystrophic muscles, regeneration gradually fails and the normal cycle of degeneration-regeneration is tipped in favor of degeneration. Currently, treatment of DMD is aimed merely at managing symptoms to improve the quality of life. By age 10, braces may be required for walking, and by age 12, most patients are confined to a wheelchair. Boys with DMD rarely live past age 20.

Sarcopenia is the degenerative loss of skeletal muscle mass and strength associated with aging (e.g., 0.5-1% loss per year after the age of 25) and is a component of the frailty syndrome. Sarcopenia is characterized first by a decrease in the size of the affected muscle, which causes weakness and frailty. As sarcopenia progresses, there is a replacement of muscle fibers with fat and an increase in fibrosis. Though the precise etiology of sarcopenia is not known, it has been theorized that the condition is caused, at least in part, by a failure in satellite cell activation, which impairs the body's ability to repair damaged muscles and properly respond to nutritional signals.

Dysferlinopathy is an autosomal recessive neuromuscular disorder caused by a deficiency of functional dysferlin protein due to mutations in the dysferlin gene. Although the role of the dysferlin protein is uncertain, a mutated dysferlin gene results in chronic muscle wasting that primarily affects limb and girdle muscles. As the muscles become atrophic, chronic fibrosis and fat accumulate. Dysferlinopathy is less severe than DMD but patients are often wheelchair bound between 30-40 years of age.

Sphingosine-1-phosphate (S1P) is a bioactive molecule with potent effects on multiple organ systems. Saba, J. D. and Hla, T. Circ. Res. 94:724-734 (2004). S1P is a signaling sphingolipid, and though some believe the compound is an intracellular secondary messenger, its mode of action is a subject of debate. Id. Researchers currently believe that S1P is formed by the phosphorylation of sphingosine, and degraded by dephosphorylation or cleavage. Its cleavage into ethanolamine phosphate and a long-chain aldehyde is reportedly catalyzed by sphingosine-1-phosphate lyase (SPL). Id.; Pyne & Pyne, Biochem J. 349:385-402 (2000).

SPL is a vitamin B₆-dependent enzyme localized in the membrane of the endoplasmic reticulum. Van Veldhoven and Mannaerts, J. Biol. Chem. 266:12502-12507 (1991); Van Veldhoven and Mannaerts, Adv. Lipid. Res. 26:69 (1993). The polynucleotide and amino acid sequences of human SPL and its gene products are described in U.S. Pat. No. 7,674,580. A component of caramel color III, 1-[5-[(1R,2S,3R)-1,2,3,4-tetrahydroxybutyl]-1H-imidazol-2-yl]-ethanone (THI), inhibits SPL activity when administered to mice. Schwab, S. et al., Science 309:1735-1739 (2005). Derivatives of THI for treatment of immunological and inflammatory diseases, including, for example, rheumatoid arthritis, are described in U.S. Pat. No. 7,825,150, U.S. Pat. No. 7,598,280, and by Bagdanoff et al. (Bagdanoff et al., J. Med. Chem, 53: 8650-8662 (2010)).

### BRIEF DESCRIPTION OF THE DRAWINGS

FIG. 1 shows that Dystrophic flies (Drosophila melanogaster) with reduced wunen have less age-dependent muscle degeneration over time. (a) Confocal images of individual indirect flight muscle (IFM) myofibrils of 3-5 and 13-15 day old flies of wild type (w), w;DysDf and w;Dys^{det1}, respectively (Actin (1st and 3rd rows) and projectin (2nd and 4th rows)). Brackets indicate width of the myofibril. (b) Graph quantifying the percentage of wild type IFM myofibrils from each genotype in (a) at 3-5 days and 13-15 days, respectively. (c) Confocal images of individual IFM myofibrils of 5 and 15 day old flies of the following genotypes: undriven UAS-wun^{RNAi}/+, tubulin-Gal4 driven Dys RNAi (TubGal4:UAS-Dys^{C-RNAi}/+), and tubulin-Gal4 driven wun RNAi, Dys RNAi double knockdown (UAS-wun^{RNAi}/TubGal4:UAS-Dys^{C-RNAi}), respectively. Actin (1st and 3rd rows) and projectin (2nd and 4th rows). Brackets indicate the width of the myofibril. Arrow indicates reduction/loss of Projectin staining at the Z-band. (d) Graph quantifying the percentage of wild type IFM myofibrils from each genotype in (c) at 5 and 15 days, respectively. (e) Activity analysis of DysDf and w (wild type) flies over 144 hours. (f) Activity analysis of tubulin- or actin-Gal4 driven Dys RNAi mutants alone and with wunen mutants (wun^{k10201}, wun^{RNAi}, wun⁴, respectively) over 72 hours. 4-5 flies (n=24-30 myofibrils) of each genotype were analyzed. For activity assays, 6 flies of each genotype were analyzed per experiment; repeated 3 times, total n=18. (*p<0.05, **p<0.01, ***p<0.001; error bars represent SEM).

FIG. 2 shows dystrophic phenotypes suppressed with altered sphingolipid metabolism gene dosage. (a) De novo sphingolipid synthesis pathway. (b) Confocal images of individual IFM myofibrils of 5 and 15 day old flies of the following genotypes: tubulin-Gal4 (TubGal4/+) tubulin-Gal4 driven Dys RNAi (TubGal4:UAS-Dys^{C-RNAi}/+), Sply, Dys double heterozygote (Sply/+; TubGal4:UAS-Dys^{C-RNAi}/+), and the Dys mutant with over-expressed lace (UASlace/+; TubGal4:UAS-Dys^{C-RNAi}/+), respectively. Actin (1st and 3rd rows) and projectin (2nd and 4th rows). Brackets indicate the width of the myofibril. Arrow indicates reduction/loss of Projectin staining at the Z-band. (c) Graph quantifying the percentage of wild type IFM myofibrils from each genotype in (b) at 5 and 15 days as well as from flies with tubulin-Gal4 driven Dys RNAi with the second chromosome balancer CyO (+/CyO; TubGal4:UAS-Dys^{C-RNAi}/+). (d) Activity analysis of tubulin-Gal4 driven Dys RNAi (TubGal4:UAS-Dys^{C-RNAi}/+) mutant alone and with reduced Sply (Sply/+; TubGal4:UAS-Dys^{C-RNAi}/+). (e) Activity analyses of the tubulin- and actin-Gal4 Dys RNAi mutants alone and with over-expressed lace (TubGal4:UAS-Dys^{C-RNAi}/+ and UAS-lace/+; TubGal4:UAS-Dys^{C-RNAi}/+) and (ActGal4: UAS-Dys^{N-RNAi}/+ and UAS-lace/ActGal4:UAS-Dys^{N-RNAi}) respectively. (f) Confocal images of individual IFM myofibrils isolated at 10 days of tubulin-Gal4 driven Dys RNAi (TubGal4:UAS-Dys^{C-RNAi}/+) mutants alone and with reduced Sply (Sply/+; TubGal4:UAS-Dys^{C-RNAi}/+) that were fed a fructose solution with and without THI. Actin (1st and 3rd rows) and projectin (2nd and 4th rows). Brackets indicate the width of the myofibril. Arrow indicates reduction/loss of Projectin staining at the Z-band. (g) Graph quantifying the percentage of wild type myofibrils from tubulin-Gal4 driven Dys RNAi (TubGal4:UAS-Dys^{C-RNAi}/+), Sply, Dys double heterozygote (Sply/+; TubGal4:UAS-Dys^{C-RNAi}/+), and wild type (w) flies fed a fructose solution with and without THI. For activity assays, 6 flies of each genotype listed on the x-axis were analyzed per experiment, repeated 3 times. Total time per experiment was 72 hours; total n=18. (*p<0.05, **p<0.01, ***p<0.001; error bars represent SEM).

FIG. 3 shows that THI administration reduces muscle fibrosis, muscle fat deposition, and dystrophy pathology following muscle injury. (a) Experimental schematic of THI (0.075 μg/day) and PBS (vehicle) treated mdx mice injected intraperitoneally (IP) twice daily for the first 72 hours following cardiotoxin (CTX) injury. Muscles, spleen or plasma from 5MO (n=6) mdx males were harvested for S1P and creatin kinase (CK) analysis at day 4 post CTX injury. Muscles from aged mdx mice (n=7 THI treated: 3 11MO females, 4 16MO males. n=6 vehicle: 3 11MO females, 3 16MO males) were harvested for histopathology analysis 18 days post CTX injury. (b-c) THI treatment significantly increased the S1P levels in spleens and CTX injured Quadriceps and decreased plasma CK. (d) Quantification of Picrosirius Red staining indicates reduced fibrosis following injury in both Tibialis Anterior (TA) and Quadriceps (Quads) muscles in mice treated with THI. (e) Representative photographs of injured Quadriceps stained with Picrosirius Red, show THI treatment reduced collagen deposition while improving overall muscle morphology and organization. Scale bars=50 μm. (f) Oil Red-O staining depicts fat deposits (arrows) over the entire cross-sectional area of THI treated and control injured TAs. Scale bars=500 μm, (g) The ratio of fat deposition in injured TAs over uninjured contralateral TAs quantified from Oil Red-O staining, was significantly reduced in THI treated vs. control animals in 11MO (*) but not 16 MO mdx mice. In contrast, the ratio of injured over uninjured fat deposits in quadriceps was significantly reduced in 16MO (#) but not in the 11MO mdx mice. *, # p<0.05, **p<0.01. Error bars represent SEM.

FIG. 4 shows that increasing S1P levels with THI increases muscle fiber size. (a) Staining for laminin and DAPI depict a dramatic increase in muscle fiber size in both injured and uninjured quadriceps with THI treatment. Accumulation of Evans blue dye in muscle fibers, injected on day 17 post injury, indicates persistent myofiber membrane damage in 11MO mice. Evans Blue positive fibers in injured quadriceps muscles (<5 over entire cross-sectional area), while almost zero were observed in uninjured contralaterals with THI treatment. In contrast >5 Evans Blue positive fibers were observed in injured and 1-5 were visible over the entire cross sectional area of uninjured quadriceps in vehicle controls. 11 MO mice depicted for fiber size increases. Scale bars=50 μm. (b-d) Quantification of minimum muscle fiber diameter reveals a significant increase in myofiber size in THI treated animals. Increased myofiber size was observed in both injured (b) and uninjured (c) Quadriceps in THI treated 11MO mdx mice whereas only uninjured Quadriceps in THI treated 16 MO mdx mice showed increased myofiber size (d) compared to vehicle controls. As indicated by the distributions, mean and median values of muscle fiber minimum diameters, there is an overall increase in muscle fiber size with THI treatment. *p<0.05, ***p<0.0005. Error bars represent SEM.

FIG. 5 shows that direct administration of S1P promotes muscle regeneration following acute injury. (a) Experimental schematic of S1P and PBS (vehicle) injected daily for the first 72 hours into TA of mdx:Myf5^{nlacZ/+} mice (n=3, left TAs injected S1P, right TAs injected PBS) following CTX injury. (b) X-gal staining reveals an increased number of β-galactosidase positive nuclei at the sites of injury in S1P treated TA muscles compared to vehicle controls. (c) Quantification of β-galactosidase+ nuclei indicates the number of Myf5+ cells is significantly increased at the site of injury in S1P treated compared to untreated flies. (d) A significant increase in β-galactosidase+ nuclei was also observed over the entire cross sectional area of each S1P treated TA muscle. (e) and (f) Staining for embryonic myosin heavy chain (eMyHC) with DAB reveals a significant increase in the number of newly regenerated muscle fibers in S1P treated TAs. (g) Quantification of the minimum diameter of the largest eMyHC+ myofibers indicates an increase in regenerated fiber size with S1P treatment. (*p<0.05, **p<0.005, ***p<0.0005; error bars represent SEM. Scale bars=50 μm).

FIG. 6 shows that administration of S1P leads to increased levels of phosphorylated ribosomal S6 in vivo. (a) Experimental schematic of S1P and PBS (vehicle) injected daily for the first 72 hours into TA of uninjured mdx mice (n=3, left TAs injected S1P, right TAs injected PBS). (b) Western blot analysis of TAs indicated neither phosphorylated Akt nor mTOR are significantly increased with S1P treatment. The levels of phosphorylated riS6 were significantly higher indicating an increased level of protein synthesis with S1P treatment. (c) Summary of experimental results indicating that S1P is a novel myogenic activator that promotes regeneration in dystrophic skeletal muscle. (*p<0.05; error bars represent SEM).

FIG. 7 shows the characterization of dystrophin mutant muscle phenotypes. (a) Confocal images of individual IFM myofibrils of 3-5 and 13-15 day old flies of w;Dys^{det1}. Actin (1st and 3rd rows) and projectin (2nd and 4th rows). Brackets indicate width of the myofibril. Arrows indicate reduction/loss of Projectin staining at the Z-band. (b) Graph quantifying the percentage of wild type staining IFM myofibrils from flies of wild type (w) and w;Dys^{det1} at 3-5 days and 13-15 days, respectively. (c) and (d) Transverse histological sections of indirect flight muscles (IFMs) viewed after H & E staining. (c) Wild type section (OregonR) from a 12 day old fly. (d) DysDf mutant section from a 12 day old fly. Arrow indicates a hole in the flight muscle indicative of the most severe degeneration. (e) Graph quantifying sectioning data. Muscle Integrity Index is a weighted average of scored sections measuring the intactness of the IFMs (see Experimental Procedures). 10 flies were scored for each experiment, each experiment was repeated 3 times. (f) and (g) Transverse histological sections of IFMs viewed after H & E staining. (f) Wild type (w) section from a 12 day old fly. (g) Dys^{det1} mutant section from a 12 day old fly. Arrow indicates a hole in the flight muscle indicative of the most severe degeneration. (h) Graph quantifying sectioning data of Dys^{det1} and w (wild type) flies. The y-axis is the Muscle Integrity Index, a weighted average of scored sections measuring the intactness of the IFMs (see Experimental Procedures). 10 flies were scored per group for this experiment. (i) Graph quantifying the climbing data. The Climbing Index is a weighted average comparing wild type flies (w) and Dystrophin mutants DysDf and Dys^{det1} (see Experimental Procedures). 20-30 flies per group were used for each experiment, which was repeated 3 times. (j) Graph comparing total activity of Dys^{det1} and w (wild type) flies over the course of 72 hrs using a Trikinetics, Inc. monitoring system. The y axis is a measure of the number of times a fly crossed an infrared light beam. Total time per experiment was 6 days. Activity was measured for 6 flies of each genotype listed on the x-axis. (**p<0.01, ***p<0.001; error bars represent SEM).

FIG. 8 shows that dystrophic flies with reduced wunen have less muscle degeneration. (a) Confocal images of individual IFM myofibrils of 5 day old flies of the following genotypes: DysDf, and the same mutant with reduced wunen (wun^{k10201} and wun⁴, respectively). Actin (top rows) and projectin (bottom rows). (b) Graph quantifying the percentage of wild type IFM myofibrils from each genotype in (a) at 5 days, respectively. (c) Climbing Index comparing 12-14 day old dystrophic flies (TubGal4:UAS-Dys^{C-RNAi}/+) and the same mutant with reduced wunen (wunk¹⁰²⁰¹, wun^{RNAi}, wun⁴, respectively). 20 flies were used per group in each experiment; repeated 3 times. (d) Activity analysis of tubulin-Gal4 driven Dys RNAi mutant and the control flies that make the composite mutant, TubGal4/+, the driver, and UAS-Dys^{C-RNAi}/+, the dsRNAi construct. (e) tubulin-Gal4 driven Dys RNAi mutant transverse section from a 12 day old fly. Arrow shows a hole in the flight muscle indicative of severe degeneration. (f) Transverse section of a 12 day old tubulin-Gal4 driven Dys RNAi mutant with reduced wunen (wun^{k10201}) in the background. At least 9 flies of each genotype were used per experiment. Each experiment was repeated 3 times. (g) Muscle Integrity Index of the tubulin-Gal4 driven Dys RNAi mutant alone and with reduced wunen (wun^{k10201}, wun^{RNAi}, wun⁴, respectively). (*p<0.05, **p<0.01, ***p<0.001; error bars represent SEM).

FIG. 9 shows that additional dystrophic phenotypes are suppressed with altered sphingolipid metabolism gene dosage. (a) Confocal images of individual IFM myofibrils of 5 day old flies of the following genotypes: DysDf, and the same mutant with reduced Sply. Actin (top rows) and projectin (bottom rows). (b) Graph quantifying the percentage of wild type IFM myofibrils from each genotype in (a) at 5 days, respectively. (c) Transverse section of a tubulin-Gal4 driven Dys RNAi 12 day old mutant fly (TubGal4:UAS-Dys^{C-RNAi}/+). (d) Transverse section of a tubulin-Gal4 driven Dys RNAi 12 day old mutant with reduced Sply (Sply/+; TubGal4:UAS-Dys^{C-RNAi}/+). (e) Transverse section of a tubulin-Gal4 driven Dys RNAi 12 day old mutant with lace over-expressed (UAS-lace/+; TubGal4:UAS-Dys^{C-RNAi}/+). (f) Graph comparing the muscle integrity index of the tubulin-Gal4 driven Dys RNAi mutant alone (TubGal4:UAS-Dys^{C-RNAi}/+), with reduced Sply (Sply/+; TubGal4:UAS-Dys^{C-RNAi}/+), and with over-expressed lace (UAS-lace/+; TubGal4:UAS-Dys^{C-RNAi}/+). 8 flies were sectioned per group in each experiment; repeated three times; total n=24. (*p<0.05, **p<0.01, ***p<0.001; error bars represent SEM).

FIG. 10 shows micrograph montages covering cross sectional areas of each TA from S1P and vehicle treated mdx4CV:Myf5^{nlacZ/+} animals. The montages were created by combing individual 10× photos. Pictures are representative of 8 μm thick, x-gal stained sections. Individual β-gal+ nuclei were counted from montages using the ImageJ v1.40 cell counter plugin. Scale bar=0.5 mm.

FIG. 11 shows the results of S1P treatment of a dysferlinopathy mouse model (A/J mice) examined following CTX induced acute injury. (a-b) Representative photographs of cross sectional areas from S1P and vehicle treated TAs from A/J mice. (c) A graph showing S1P treatment of CTX injured TA muscles in the A/J mice significantly decreased the average percentage of fibrosis.

FIG. 12 shows the beneficial effects of THI administration on muscle function in dystrophic mice. (a) Graph of the specific maximum force of the muscle in vehicle treated and THI-treated muscle. (b) Graph of the muscle force measured at elevating frequency. (c) Graph of muscle fatigue with stimulation. (d) Graph of muscle recovery after fatigue.

FIG. 13 shows a graph of myofibril analysis after administration of THI-oxime and negative control to dystrophic Drosophila.

FIG. 14 shows a graph of myofibril analysis after administration of FTY720 and negative control to dystrophic Drosophila.

FIG. 15 shows a graph of myofibril analysis after administration of THI and without administration of THI to dystrophic Drosophila.

FIG. 16 is a graph of the ratio of dry weight from each CTX injured over uninjured muscles (TAs and quadriceps) normalized to body weight of 16MO mdx male mice. *p<0.05. Error bars represent SEM.

FIG. 17 shows a graph of diaphragm muscle diameters of THI treated mice. As indicated by the distributions, mean and median values of muscle fiber minimum diameters, there is an overall increase in muscle fiber size with THI treatment. *p<0.05.

FIG. 18 shows an increase in satellite cells in THI-treated mice muscles. (a) Photographs showing staining for Pax7 (AlexaFluor488 depicted in green) revealing a greater number of satellite cells, collectively in THI-treated limb muscles. Representative nuclear Pax7 staining of CTX injured TA muscles from THI (left column) and Vehicle treated (right column) mdx animals. Arrowheads designate Pax7+ nuclei. Scale bar=50 μm. (b) Graph showing quantification of Pax7+ nuclei reveals increase in satellite cells. Error bars represent SEM.

FIG. 19 shows that the increased number of the Myf5+ cells observed with S1P treatment are mainly activated satellite cells. (a) Photographs showing staining for myogenin (AlexaFluor488 depicted in green) revealing differentiating myogenic cells with S1P treatment. Representative staining of myogenin localized to cell nuclei, in CTX injured TA muscles from S1P (left column) and vehicle treated (right column) mdx4CV:Myf5^{nlacZ/+} mice. Arrowheads designate myogenin+ nuclei. Scale bar=50 μm. (b) Graph showing quantification of myogenin+ nuclei indicates a greater number of differentiating myogenic cells between S1P and vehicle treated TAs. Error bars represent SEM.

FIG. 20 shows the beneficial effects of THI-oxime administration on muscle function in dystrophic mice.

### DETAILED DESCRIPTION OF PREFERRED EMBODIMENTS

The current disclosure provides compositions for treating degenerative muscle conditions. The compositions described herein are sphingosine-1-phosphate (S1P) promoting compositions that include one or more S1P promoting agents. In particular embodiments, the S1P promoting compositions are prepared as pharmaceutical compositions and include one or more S1P promoting agents in combination with a pharmaceutically acceptable carrier. The S1P promoting agent included in the compositions described herein may be S1P, an S1P derivative, an S1P molecule or internal agonist, or a compound that increases S1P levels. In some embodiments, the S1P promoting agent included in the compositions described herein may be a compound that simulates or imitates S1P activity such as a sphingosine analog or a S1P receptor agonist. Alternatively, the S1P promoting agent included in the compositions described herein may be an inhibitor of sphingosine-1-phosphate lyase (SPL). In specific embodiments, the SPL inhibitor is 1-[5-[(1R,2S,3R)-1,2,3,4-tetrahydroxybutyl]-1H-imidazol-2-yl]-ethanone (THI). In other embodiments, the SPL inhibitor is selected from one or more of the THI derivatives described herein. The S1P promoting compositions described herein may include a combination of two or more S1P promoting agents, including a combination of S1P and one or more SPL inhibitors or a combination of two or more SPL inhibitors as described herein.

Methods for treating degenerative muscle conditions are also provided. In some embodiments, the methods include inhibiting muscle degeneration in a subject suffering from a degenerative muscle condition. In other embodiments, the methods include promoting muscle repair or regeneration in a subject suffering from a degenerative muscle condition. In still other embodiments, methods for promoting proliferation of satellite cells in muscle tissue of a subject suffering from a degenerative muscle condition are described. In yet further embodiments, the methods described herein include methods for promoting an increase in muscle fiber number, size or dimension in a subject suffering from a degenerative muscle condition. In still further embodiments, the methods described herein include methods for inhibiting fat deposition in muscle tissue of a subject suffering from a degenerative muscle condition. In still further embodiments, the methods described herein include methods for inhibiting fibrosis in muscle tissue of a subject suffering from a degenerative muscle condition. In each embodiment of the methods described herein, a therapeutically effective amount of an S1P promoting composition according to the present description is administered to the subject. The degenerative muscle condition treated by the methods described herein may be selected from, for example, muscular dystrophy, sarcopenia, muscle injury, including acute muscle injury, resulting in loss of muscle tissue, muscle atrophy, wasting or degeneration, muscle overuse, muscle disuse atrophy, denervation muscle atrophy, dysferlinopathy, AIDS/HIV, diabetes, chronic obstructive pulmonary disease, kidney disease, cancer, aging, autoimmune disease, polymyositis, and dermatomyositis. In specific embodiments of the methods described herein, the degenerative muscle condition treated is selected from Duchenne Muscular Dystrophy (DMD) and Becker Muscular Dystrophy (BMD).

### I. DEFINITIONS

The terminology used herein is for the purpose of describing particular embodiments only, and is not intended to limit the scope of the present invention which will be limited only by the appended claims. Unless defined otherwise, all technical and scientific terms used herein have the meanings that would be commonly understood by one of skill in the art in the context of the present specification.

As used herein, the singular forms “a”, “an” and “the” are intended to include the plural forms as well, unless the context clearly indicates otherwise. Also, as used herein, “and/or” refers to and encompasses any and all possible combinations of one or more of the associated listed items. Furthermore, the term “about,” as used herein when referring to a measurable value such as an amount, dose, time, temperature, and the like, is meant to encompass variations of 20%, 10%, 5%, 1%, 0.5%, or even 0.1% of the specified amount.

Unless otherwise indicated, the term “include” has the same meaning as “include, but are not limited to,” the term “includes” has the same meaning as “includes, but is not limited to,” and the term “including” has the same meaning as “including, but not limited to,” Similarly, the term “such as” has the same meaning as the term “such as, but not limited to.”

As used herein, the term “subject” refers to an animal or human, preferably a mammal, subject in need of treatment for a given disease, disorder, condition, or injury.

The term “pharmaceutically acceptable” refers to materials approved by a regulatory agency, such as by a regulatory agency of a Federal or a state government, or listed in the U.S. Pharmacopeia or other generally recognized pharmacopeia for use in a subject.

The terms “inhibit,” “inhibiting,” and “inhibition” refer to decrease in an activity, response, condition, or other biological parameter, including the production, presence, expression, or function of cells, biomolecules or bioactive molecules. The terms “inhibit,” “inhibiting,” and “inhibition” include, but are not limited to, the complete ablation of an activity, response, or condition, as well as the complete ablation of the production, presence, or expression of cells, biomolecules, or bioactive molecules. The terms “inhibit,” “inhibiting,” and “inhibition” may also include a measurable reduction in an activity, response, or condition, or a measurable reduction in the production, presence, or expression of cells, biomolecules, or bioactive molecules, as compared to a native or control level.

The terms “inhibitor of SPL” or “SPL inhibitor” refer to agents and compositions that directly or indirectly inhibit activity, function, expression, production, or maintenance of SPL in vivo. SPL inhibition may be evidenced by promotion of activity, function, expression, production, or maintenance of S1P in vivo.

The terms “promote,” “promotion,” and “promoting” refer to an increase in an activity, response, condition, or other biological parameter, including the production, presence, expression, or function of cells, biomolecules or bioactive molecules. The terms “promote,” “promotion,” and “promoting include, but are not limited to, initiation of an activity, response, or condition, as well as initiation of the production, presence, or expression of cells, biomolecules, or bioactive molecules. The terms “promote,” “promotion,” and “promoting” may also include measurably increasing an activity, response, or condition, or measurably increasing the production, presence, expression, or function of cells, biomolecules, or bioactive molecules, as compared to a native or control level.

The term “S1P promoting composition” refers to compositions including one or more S1P promoting agents. For example, an S1P promoting composition is a composition that increases S1P levels by an amount ranging from at least about 5% increase to at least about 200%. In certain examples, an S1P promoting composition is a composition that increases S1P levels by at least about 5%, 10%, 15%, 20%, 25%, 30%, 35%, 40%, 45%, 50%, 60%, 70%, 80%, 90%, 100%, 110%, 120%, 130%, 140%, 150%, 160%, 170%, 180%, 190%, and 200%, or more. In further examples, an S1P promoting composition is a composition that increases S1P levels by an amount ranging from at least about a 2-fold increase to at least about a 20-fold increase. In particular examples, an S1P promoting composition is a composition that increases S1P levels by about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, and 20-fold, or more. Alternatively or in addition to, in certain embodiments, S1P promoting compositions as described herein include compositions that include one or more S1P promoting agents capable of one or more of the following, as described in further detail herein below: promoting proliferation of satellite cells; promoting muscle regeneration; promoting an increase in muscle fiber size; inhibiting fat deposition in muscle tissue; and inhibiting formation of fibrosis in muscle tissue.

The term “S1P promoting agent” refers to agents that promote the activity, function, expression, production, or maintenance of S1P in viva For example, an S1P promoting agent is an agent that increases S1P levels, an agent that prevents the degradation or de-phosphorylation of S1P, agents that are sphingosine analogs or S1P receptor agonists that activate S1P receptors or intracellular mediated signaling, and the like. Alternatively or in addition to, in certain embodiments, S1P promoting agents as described herein include agents capable of one or more of the following, as described in further detail herein below: promoting proliferation of satellite cells; promoting muscle regeneration; promoting an increase in muscle fiber size; inhibiting fat deposition in muscle tissue; and inhibiting formation of fibrosis in muscle tissue.

The term “carrier” means a compound, composition, substance, or structure that, when in combination with an S1P promoting agent or S1P promoting composition, aids or facilitates preparation, storage, administration, delivery, efficacy, selectivity, or any other feature of the agent or composition for its intended use or purpose. For example, a carrier can be selected to minimize any degradation of the active ingredient and to minimize any adverse side effects in the subject. Further as used herein, “carrier” includes pharmaceutically acceptable carriers, vehicles, adjuvants, diluents, surfactants, excipients, permeation enhancers, stabilizers, or any combination thereof, with which an S1P promoting agent as described herein may be combined to provide a pharmaceutical composition suitable for administration to a subject.

As used herein, the terms “treat,” “treating,” and “treatment” refer to a therapeutic benefit, whereby the detrimental effect(s) or progress of a particular disease, disorder, condition, or injury is prevented, reduced, halted, inhibited or reversed. The terms “treat,” “treating,” and “treatment” also refer to promoting a response, condition, other biological parameter, whereby the detrimental effect(s) or progress of a particular disease, condition, or injury is prevented, reduced, halted, inhibited or reversed. For example, treating a degenerative muscle condition as contemplated herein includes, but is not limited to, one or more of the following: promoting local or systemic increases in S1P; inhibiting degeneration of muscle tissue; promoting regeneration of muscle tissue; promoting satellite cell proliferation in muscle tissue; inhibiting fibrosis in muscle tissue; inhibiting fat deposition in muscle tissue; and promoting increases in muscle fiber size or number.

A “therapeutically effective” refers to an amount sufficient to treat a disease, disorder, condition, or injury.

As used herein, the term “satellite cells” refers to small mononuclear progenitor cells found in mature muscle that are able to differentiate to augment existing muscle fibers and form new muscle fibers. Satellite cells are involved in the normal growth of muscle, as well as muscle regeneration following injury, degeneration, or disease.

The term “degenerative muscle condition” refers to conditions, disorders, diseases and injuries characterized by one or more of muscle loss, muscle degeneration or wasting, muscle weakness, and defects or deficiencies in proteins associated with normal muscle function, growth or maintenance. In certain embodiments, a degenerative muscle condition is sarcopenia or cachexia. In other embodiments, a degenerative muscle condition is one or more of muscular dystrophy, muscle injury, including acute muscle injury, resulting in loss of muscle tissue, muscle atrophy, wasting or degeneration, muscle overuse, muscle disuse atrophy, muscle disuse atrophy, denervation muscle atrophy, dysferlinopathy, AIDS/HIV, diabetes, chronic obstructive pulmonary disease, kidney disease, cancer, aging, autoimmune disease, polymyositis, and dermatomyositis.

As used herein, “muscular dystrophy” includes, for example, Duchenne, Becker, Limb-girdle, Congenital, Facioscapulohumeral, Myotonic, Oculopharyngeal, Distal, and Emery-Dreifuss muscular dystrophies. In particular embodiments, the muscular dystrophy is characterized, at least in part, by a deficiency or dysfunction of the protein dystrophin. Such muscular dystrophies include Duchenne Muscular Dystrophy (DMD), and Becker Muscular Dystrophy (DMD). In other embodiments, the muscular dystrophy is associated with degenerative muscle conditions such as muscle disuse atrophy, denervation muscle atrophy, dysferlinopathy, AIDS/HIV, diabetes, chronic obstructive pulmonary disease, kidney disease, cancer, aging, autoimmune disease, polymyositis, and dermatomyositis.

The term “alkenyl” refers to a straight chain, branched and/or cyclic hydrocarbon having from 2 to 20 (e.g., 2 to 10 or 2 to 6) carbon atoms, and including at least one carbon-carbon double bond. Representative alkenyl moieties include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl and 3-decenyl.

The term “alkyl” refers to a straight chain, branched and/or cyclic (“cycloalkyl”) hydrocarbon having from 1 to 20 (e.g., 1 to 10 or 1 to 4) carbon atoms. Alkyl moieties having from 1 to 4 carbons are referred to as “lower alkyl.” Examples of alkyl groups include, but are not limited to, methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl and dodecyl. Cycloalkyl moieties may be monocyclic or multicyclic, and examples include cyclopropyl, cyclobutyl, cyclopentyl, cyclohexyl, and adamantyl. Additional examples of alkyl moieties have linear, branched and/or cyclic portions (e.g., 1-ethyl-4-methyl-cyclohexyl). The term “alkyl” includes saturated hydrocarbons as well as alkenyl and alkynyl moieties.

The term “alkylaryl” or “alkyl-aryl” refers to alkyl moiety bound to an aryl moiety.

The terms “alkylheteroaryl” or “alkyl-heteroaryl” refer to an alkyl moiety bound to a heteroaryl moiety.

The term “alkylheterocycle” or “alkyl-heterocycle” refer to an alkyl moiety bound to a heterocycle moiety.

The term “alkynyl” refers to a straight chain, branched or cyclic hydrocarbon having from 2 to 20 (e.g., 2 to 20 or 2 to 6) carbon atoms, and including at least one carbon-carbon triple bond. Representative alkynyl moieties include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl and 9-decynyl.

The term “alkoxy” refers to an —O-alkyl group. Examples of alkoxy groups include, but are not limited to, —OCH₃, —OCH₂CH₃, —O(CH₂)₂CH₃, —O(CH₂)₃CH₃, —O(CH₂)₄CH₃, and —O(CH₂)₅CH₃.

The term “aryl” refers to an aromatic ring or an aromatic or partially aromatic ring system composed of carbon and hydrogen atoms. An aryl moiety may comprise multiple rings bound or fused together. Examples of aryl moieties include, but are not limited to, anthracenyl, azulenyl, biphenyl, fluorenyl, indan, indenyl, naphthyl, phenanthrenyl, phenyl, 1,2,3,4-tetrahydro-naphthalene, and tolyl.

The term “arylalkyl” or “aryl-alkyl” refers to an aryl moiety bound to an alkyl moiety.

The terms “halogen” and “halo” encompass fluorine, chlorine, bromine, and iodine.

The term “heteroalkyl” refers to an alkyl moiety (e.g., linear, branched or cyclic) in which at least one of its carbon atoms has been replaced with a heteroatom (e.g., N, O or S).

The term “heteroaryl” means an aryl moiety wherein at least one of its carbon atoms has been replaced with a heteroatom (e.g., N, O or S). Examples include, but are not limited to, acridinyl, benzimidazolyl, benzofuranyl, benzoisothiazolyl, benzoisoxazolyl, benzoquinazolinyl, benzothiazolyl, benzoxazolyl, furyl, imidazolyl, indolyl, isothiazolyl, isoxazolyl, oxadiazolyl, oxazolyl, phthalazinyl, pyrazinyl, pyrazolyl, pyridazinyl, pyridyl, pyrimidinyl, pyrimidyl, pyrrolyl, quinazolinyl, quinolinyl, tetrazolyl, thiazolyl, and triazinyl.

The term “heteroarylalkyl” or “heteroaryl-alkyl” refers to a heteroaryl moiety bound to an alkyl moiety.

The term “heterocycle” refers to an aromatic, partially aromatic or non-aromatic monocyclic or polycyclic ring or ring system comprised of carbon, hydrogen and at least one heteroatom (e.g., N, O or S). A heterocycle may comprise multiple (i.e., two or more) rings fused or bound together. Heterocycles include heteroaryls. Examples include, but are not limited to, benzo[1,3]dioxolyl, 2,3-dihydro-benzo[1,4]dioxinyl, cinnolinyl, furanyl, hydantoinyl, morpholinyl, oxetanyl, oxiranyl, piperazinyl, piperidinyl, pyrrolidinonyl, pyrrolidinyl, tetrahydrofuranyl, tetrahydropyranyl, tetrahydropyridinyl, tetrahydropyrimidinyl, tetrahydrothiophenyl, tetrahydrothiopyranyl and valerolactamyl.

The terms “heterocyclealkyl” or “heterocycle-alkyl” refer to a heterocycle moiety bound to an alkyl moiety.

The term “heterocycloalkyl” refers to a non-aromatic heterocycle.

The terms “heterocycloalkylalkyl” or “heterocycloalkyl-alkyl” refer to a heterocycloalkyl moiety bound to an alkyl moiety.

As used herein, “pharmaceutically acceptable salts” refer to salts prepared from pharmaceutically acceptable non-toxic acids or bases including inorganic acids and bases and organic acids and bases. Suitable pharmaceutically acceptable base addition salts include, but are not limited to, metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art. See, e.g., Remington's Pharmaceutical Sciences (18th ed., Mack Publishing, Easton Pa.: 1990) and Remington: The Science and Practice of Pharmacy (19th ed., Mack Publishing, Easton Pa.: 1995).

The term “stereoisomeric mixture” encompasses racemic mixtures as well as stereometrically enriched mixtures. Stereometrically enriched mixtures refer to mixtures wherein relatively more of one stereoisomer of a compound is present (e.g., mixtures where the relative percentages of R/S stereoisomers are selected from 30/70, 35/65, 40/60, 45/55, 55/45, 60/40, 65/35 and 70/30).

Unless otherwise indicated, the term “stereomerically pure” means a composition that comprises one stereoisomer of a compound and is substantially free of other stereoisomers of that compound. For example, a stereomerically pure composition of a compound having one stereocenter will be substantially free of the opposite stereoisomer of the compound. A stereomerically pure composition of a compound having two stereocenters will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound.

The term “substituted,” when used to describe a chemical structure or moiety, refers to a derivative of that structure or moiety wherein one or more of its hydrogen atoms is substituted with an atom, chemical moiety or functional group such as, but not limited to, alcohol, aldehylde, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), amidinyl (—C(NH)NH-alkyl or —C(NR)NH₂), amine (primary, secondary and tertiary such as alkylamino, arylamino, arylalkylamino), aroyl, aryl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carbonyl, carboxyl, carboxylic acid, carboxylic acid anhydride, carboxylic acid chloride, cyano, ester, epoxide, ether (e.g., methoxy, ethoxy), guanidino, halo, haloalkyl (e.g., —CCl₃, —CF₃, —C(CF₃)₃), heteroalkyl, hemiacetal, imine (primary and secondary), isocyanate, isothiocyanate, ketone, nitrile, nitro, oxygen (i.e., to provide an oxo group), phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) and urea (—NHCONH-alkyl-).

One or more adjectives immediately preceding a series of nouns are to be construed as applying to each of the nouns. For example, the phrase “optionally substituted alkyl, aryl, or heteroaryl” has the same meaning as “optionally substituted alkyl, optionally substituted aryl, or optionally substituted heteroaryl.”

A chemical moiety that forms part of a larger compound may be described herein using a name commonly accorded it when it exists as a single molecule or a name commonly accorded its radical. For example, the terms “pyridine” and “pyridyl” are accorded the same meaning when used to describe a moiety attached to other chemical moieties. Thus, the two phrases “XOH, wherein X is pyridyl” and “XOH, wherein X is pyridine” are accorded the same meaning, and encompass the compounds pyridin-2-ol, pyridin-3-ol and pyridin-4-ol.

If the stereochemistry of a structure or a portion of a structure is not indicated with, for example, bold or dashed lines, the structure or the portion of the structure is to be interpreted as encompassing all stereoisomers of it. Moreover, any atom shown in a drawing with unsatisfied valences is assumed to be attached to enough hydrogen atoms to satisfy the valences. In addition, chemical bonds depicted with one solid line parallel to one dashed line encompass both single and double (e.g., aromatic) bonds, if valences permit.

### II. S1P PROMOTING COMPOSITIONS

The S1P promoting compositions provided herein are compositions that include an S1P promoting agent. In some embodiments, an S1P promoting composition is provided as a pharmaceutical composition that includes an S1P promoting agent in combination with a pharmaceutically acceptable carrier. The S1P promoting compositions described herein may include a single S1P promoting agent or a combination of two or more S1P promoting agents, with the S1P promoting agents used in such embodiments being selected from those described herein.

In some embodiments of the S1P promoting compositions described herein, S1P itself is included as an S1P promoting agent. Where included in the compositions described herein, S1P may be provided as a compound according to Formula I:

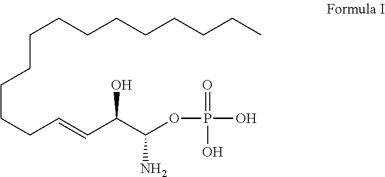 [SMILES: CCCCCCCCCCCCC/C=C/[C@@H](O)[C@@H](N)OP(=O)(O)O]

including any pharmaceutically acceptable salts, esters, isomers or solvates (e.g., hydrates) thereof.

In other embodiments, S1P promoting agents suitable for use in the S1P promoting compositions described herein includes inhibitors of SPL. In one such embodiment, the SPL inhibitor is 1-[5-[(1R,2S,3R)-1,2,3,4-tetrahydroxybutyl]-1H-imidazol-2-yl]-ethanone, also known as 2-acetyl-5-tetrahydroxybutyl imidazole, or THI (Cayman Chemical, Mich.). Where included in the compositions described herein, THI may be provided as a compound according to Formula II:

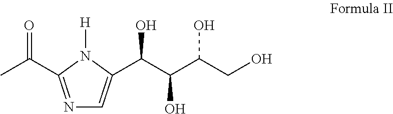 [SMILES: CC(=O)c1ncc([C@@H](O)[C@H](O)[C@H](O)CO)[nH]1]

including any pharmaceutically acceptable salts, esters, isomers or solvates (e.g., hydrates) thereof. THI may be synthesized using methods known by those of skill in the art, such as those methods described in Halweg, K. M. and Büchi, G., J. Org. Chem. 50:1134-1136 (1985) and Bagdanoff et al., J. Med. Chem. 52:3941-3953 (2009), incorporated by reference herein. In alternative embodiments, SPL inhibitors suitable for use as an S1P promoting agent may be selected from biologically active derivatives of THI. For purposes of the present disclosure, a biologically active derivative of THI is a compound that is an SPL inhibitor and/or an S1P promoting agent.

In one embodiment, biologically active derivatives of THI are compounds according to Formula III:

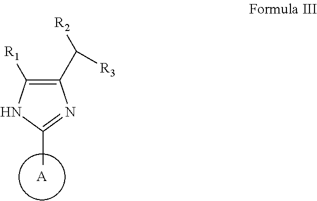

including any pharmaceutically acceptable salts, esters, isomers or solvates (e.g., hydrates) thereof, wherein: A is an optionally substituted heterocycle; R₁ is OR_{1A}, OC(O)R_{1A}, C(O)OR_{1A}, hydrogen, halogen, nitrile, or optionally substituted alkyl, aryl, alkylaryl, arylalkyl, heteroalkyl, heterocycle, alkylheterocycle, or heterocyclealkyl; R₂ is OR_{2A}, OC(O)R_{2A}, hydrogen, halogen, or optionally substituted alkyl, aryl, alkylaryl, arylalkyl, heteroalkyl, heterocycle, alkylheterocycle, or heterocyclealkyl; R₃ is N(R_{3A})₂, hydrogen, hydroxy, or optionally substituted alkyl, aryl, alkylaryl, arylalkyl, heteroalkyl, heterocycle, alkylheterocycle, or heterocyclealkyl; and each of R_{1A}, R_{2A}, and R_{3A} is independently hydrogen or optionally substituted alkyl, aryl, alkylaryl, arylalkyl, heteroalkyl, heterocycle, alkylheterocycle, or heterocyclealkyl.

In particular embodiments, biologically active THI derivatives according to Formula III are compounds according to Formula III(a) or Formula III(b):

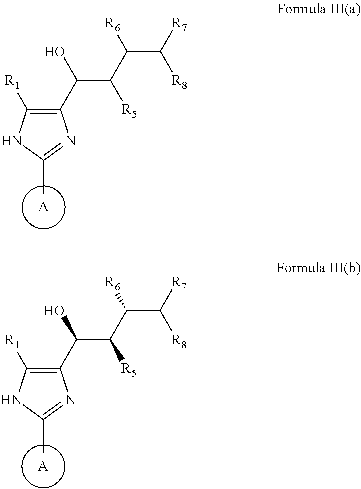

including any pharmaceutically acceptable salts, esters, isomers or solvates (e.g., hydrates) thereof, wherein: R₅ is OR_{5A}, OC(O)R_{5A}, N(R_{5B})₂, NHC(O)R_{5B}, hydrogen, or halogen; R₆ is OR_{6A}, OC(O)R_{6A}, N(R_{6B})₂, NHC(O)R_{6B}, hydrogen, or halogen; R₇ is OR_{7A}, OC(O)R_{7A}, N(R_{7B})₂, NHC(O)R_{7B}, hydrogen, or halogen; R₈ is CH₂OR_{8A}, CH₂OC(O)R_{8A}, N(R_{8B})₂, NHC(O)R_{8B}, hydrogen, or halogen; each of R_{1A}, R_{5A}, R_{6A}, R_{7A}, and R_{8A} is independently hydrogen or optionally substituted alkyl, aryl, alkylaryl, arylalkyl, heteroalkyl, heterocycle, alkylheterocycle, or heterocyclealkyl; and each of R_{5B}, R_{6B}, R_{7B} and R_{8B} is independently hydrogen or alkyl optionally substituted with one or more hydroxy or halogen groups.

In other embodiments, biologically active THI derivatives are compounds according to Formula IV:

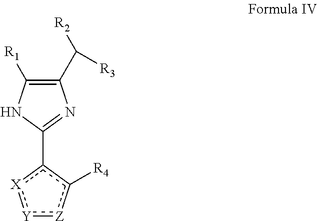

including any pharmaceutically acceptable salts, esters, isomers or solvates (e.g., hydrates) thereof, wherein: X is CR₄, CHR₄, N, NR₉, O or S; Y is CR₄, CHR₄, N, NR₉, O or S; Z is CR₄, CHR₄, N, NR₉, O or S; R₁ is OR_{1A}, C(O)OR_{1A}, hydrogen, halogen, nitrile, or optionally substituted alkyl, aryl, alkylaryl, arylalkyl, heteroalkyl, heterocycle, alkylheterocycle, or heterocyclealkyl; R₂ is OR_{2A}, OC(O)R_{2A}, hydrogen, halogen, or optionally substituted alkyl, aryl, alkylaryl, arylalkyl, heteroalkyl, heterocycle, alkylheterocycle, or heterocyclealkyl; R₃ is N(R_{3A})₂, hydrogen, hydroxy, or optionally substituted alkyl, aryl, alkylaryl, arylalkyl, heteroalkyl, heterocycle, alkylheterocycle, or heterocyclealkyl; each of R_{1A}, R_{2A}, and R_{3A} is independently hydrogen or optionally substituted alkyl, aryl, alkylaryl, arylalkyl, heteroalkyl, heterocycle, alkylheterocycle, or heterocyclealkyl; each R₄ is independently OR_{4A}, OC(O)R_{4A}, hydrogen, halogen, or optionally substituted alkyl, aryl, alkylaryl, arylalkyl, heteroalkyl, heterocycle, alkylheterocycle, or heterocyclealkyl; each R₉ is independently hydrogen or optionally substituted alkyl, aryl, alkylaryl, arylalkyl, heteroalkyl, heterocycle, alkylheterocycle, or heterocyclealkyl; and each of R_{1A}, R_{2A}, R_{3A} and R_{4A} is independently hydrogen or optionally substituted alkyl, aryl, alkylaryl, arylalkyl, heteroalkyl, heterocycle, alkylheterocycle, or heterocyclealkyl.

In particular embodiments, biologically active THI derivatives according to Formula IV are compounds according to Formula IV(a) or Formula IV(b):

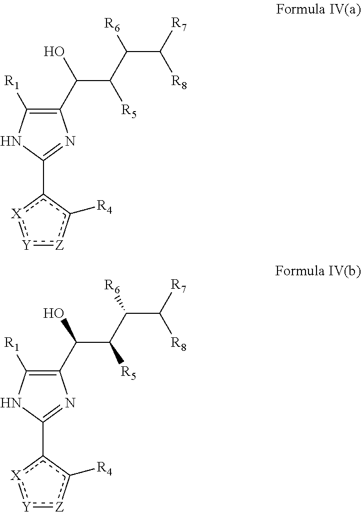

including any pharmaceutically acceptable salts, esters, isomers or solvates (e.g., hydrates) thereof, wherein: R₅ is OR_{5A}, OC(O)R_{5A}, N(R_{5B})₂, NHC(O)R_{5B}, hydrogen, or halogen; R₆ is OR_{6A}, OC(O)R_{6A}, N(R_{6B})₂, NHC(O)R_{6B}, hydrogen, or halogen; R₇ is OR_{7A}, OC(O)R_{7A}, N(R_{7B})₂, NHC(O)R_{7B}, hydrogen, or halogen; R₈ is CH₂OR_{8A}, CH₂OC(O)R_{8A}, N(R_{8B})₂, NHC(O)R_{8B}, hydrogen, or halogen; each of R_{1A}, R_{5A}, R_{6A}, R_{7A}, and R_{8A} is independently hydrogen or optionally substituted alkyl, aryl, alkylaryl, arylalkyl, heteroalkyl, heterocycle, alkylheterocycle, or heterocyclealkyl; and each of R_{5B}, R_{6B}, R_{7B} and R_{8B} is independently hydrogen or alkyl optionally substituted with one or more hydroxy or halogen groups.

In other embodiments, biologically active THI derivatives are compounds according to Formula V:

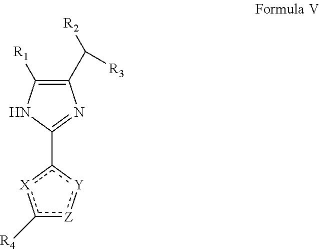

including any pharmaceutically acceptable salts, esters, isomers or solvates (e.g., hydrates) thereof, wherein: X is CR₄, CHR₄, N, NR₉, O or S; Y is CR₄, CHR₄, N, NR₉, O or S; Z is CR₄, CHR₄, N, NR₉, O or S; R₁ is OR_{1A}, C(O)OR_{1A}, hydrogen, halogen, nitrile, or optionally substituted alkyl, aryl, alkylaryl, arylalkyl, heteroalkyl, heterocycle, alkylheterocycle, or heterocyclealkyl; R₂ is OR_{2A}, OC(O)R_{2A}, hydrogen, halogen, or optionally substituted alkyl, aryl, alkylaryl, arylalkyl, heteroalkyl, heterocycle, alkylheterocycle, or heterocyclealkyl; R₃ is N(R_{3A})₂, hydrogen, hydroxy, or optionally substituted alkyl, aryl, alkylaryl, arylalkyl, heteroalkyl, heterocycle, alkylheterocycle, or heterocyclealkyl; each R₄ is independently OR_{4A}, OC(O)R_{4A}, hydrogen, halogen, or optionally substituted alkyl, aryl, alkylaryl, arylalkyl, heteroalkyl, heterocycle, alkylheterocycle, or heterocyclealkyl; each R₉ is independently hydrogen or optionally substituted alkyl, aryl, alkylaryl, arylalkyl, heteroalkyl, heterocycle, alkylheterocycle, or heterocyclealkyl; and each of R_{1A}, R_{2A}, R_{3A} and R_{4A} is independently hydrogen or optionally substituted alkyl, aryl, alkylaryl, arylalkyl, heteroalkyl, heterocycle, alkylheterocycle, or heterocyclealkyl.

In particular embodiments, biologically active THI derivatives according to Formula V are compounds according to Formula V(a) or Formula V(b):

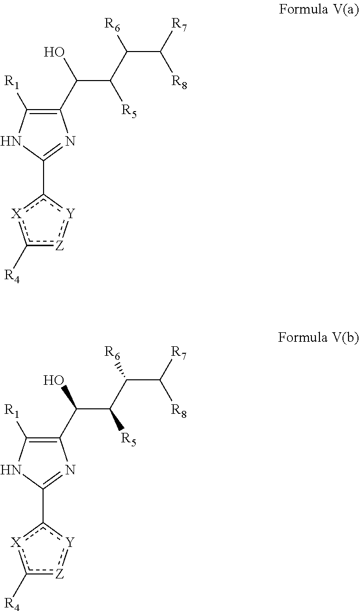

including any pharmaceutically acceptable salts, esters, isomers or solvates (e.g., hydrates) thereof, wherein: R₅ is OR_{5A}, OC(O)R_{5A}, N(R_{5B})₂, NHC(O)R_{5B}, hydrogen, or halogen; R₆ is OR_{6A}, OC(O)R_{6A}, N(R_{6B})₂, NHC(O)R_{6B}, hydrogen, or halogen; R₇ is OR_{7A}, OC(O)R_{7A}, N(R_{7B})₂, NHC(O)R_{7B}, hydrogen, or halogen; R₈ is CH₂OR_{8A}, CH₂OC(O)R_{8A}, N(R_{8B})₂, NHC(O)R_{8B}, hydrogen, or halogen; each of R_{1A}, R_{5A}, R_{6A}, R_{7A}, and R_{8A} is independently hydrogen or optionally substituted alkyl, aryl, alkylaryl, arylalkyl, heteroalkyl, heterocycle, alkylheterocycle, or heterocyclealkyl; and each of R_{5B}, R_{6B}, R_{7B} and R_{8B} is independently hydrogen or alkyl optionally substituted with one or more hydroxy or halogen groups.

In other embodiments, biologically active THI derivatives are compounds according to Formula VI:

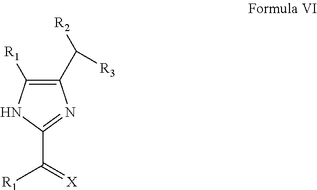

including any pharmaceutically acceptable salts, esters, isomers or solvates (e.g., hydrates) thereof, wherein: X is O or NR₃; R₁ is OR_{1A}, NHOH, hydrogen, or optionally substituted alkyl, aryl, alkylaryl, arylalkyl, heteroalkyl, heterocycle, alkylheterocycle, or heterocyclealkyl; R₂ is OR_{2A}, C(O)OR_{2A}, hydrogen, halogen, nitrile, or optionally substituted alkyl, aryl, alkylaryl, arylalkyl, heteroalkyl, heterocycle, alkylheterocycle, or heterocyclealkyl; R₃ is OR_{3A}, N(R_{3A})₂, NHC(O)R_{3A}, NHSO₂R_{3A}, or hydrogen; R₄ is OR_{4A}, OC(O)R_{4A}, hydrogen, halogen, or optionally substituted alkyl, aryl, alkylaryl, arylalkyl, heteroalkyl, heterocycle, alkylheterocycle, or heterocyclealkyl; R₅ is N(R_{5A})₂, hydrogen, hydroxy, or optionally substituted alkyl, aryl, alkylaryl, arylalkyl, heteroalkyl, heterocycle, alkylheterocycle, or heterocyclealkyl; and each of R_{1A}, R_{2A}, R_{3A}, R_{4A}, and R_{5A} is independently hydrogen or optionally substituted alkyl, aryl, alkylaryl, arylalkyl, heteroalkyl, heterocycle, alkylheterocycle, or heterocyclealkyl.

Particular compounds of formula VI are such that if X is O; R₁ is alkyl of 1 to 4 carbons, phenyl, benzyl or phenylethyl; R₂ is hydrogen; and one of R₄ and R₅ is hydroxyl; the other of R₄ and R₅ is not alkyl of 1 to 6 carbons, hydroxyalkyl of 1 to 6 carbons, polyhydroxyalkyl of 1 to 6 carbons having up to one hydroxyl per carbon, polyacetylalkyl of 1 to 6 carbons having up to one acetyl per carbon, phenyl, benzyl or phenylethyl.

In other embodiments, biologically active THI derivatives are compounds according to Formula VII:

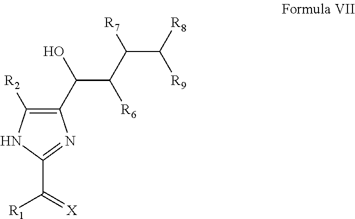

including any pharmaceutically acceptable salts, esters, isomers or solvates (e.g., hydrates) thereof, wherein: X is O or NR₃; R₁ is OR_{1A}, NHOH, hydrogen, or optionally substituted alkyl, aryl, alkylaryl, arylalkyl, heteroalkyl, heterocycle, alkylheterocycle, or heterocyclealkyl; R₂ is OR_{2A}, C(O)OR_{2A}, hydrogen, halogen, nitrile, or optionally substituted alkyl, aryl, alkylaryl, arylalkyl, heteroalkyl, heterocycle, alkylheterocycle, or heterocyclealkyl; R₃ is OR_{3A}, N(R_{3A})₂, NHC(O)R_{3A}, NHSO₂R_{3A}, or hydrogen; R₆ is OR_{6A}, OC(O)R_{6A}, N(R_{6B})₂, NHC(O)R_{6B}, hydrogen, or halogen; R₇ is OR_{7A}, OC(O)R_{7A}, N(R_{7B})₂, NHC(O)R_{7B}, hydrogen, or halogen; R₈ is OR_{8A}, OC(O)R_{8A}, N(R_{8B})₂, NHC(O)R_{8B}, hydrogen, or halogen; R₉ is CH₂OR_{9A}, CH₂OC(O)R_{9A}, N(R_{9B})₂, NHC(O)R_{9B}, hydrogen, or halogen; each of R_{1A}, R_{2A}, R_{3A}, R_{6A}, R_{7A}, R_{8A} and R_{9A} is independently hydrogen or optionally substituted alkyl, aryl, alkylaryl, arylalkyl, heteroalkyl, heterocycle, alkylheterocycle, or heterocyclealkyl; and each of R_{6B}, R_{7B}, R_{8B} and R_{9B} is independently hydrogen or alkyl optionally substituted with one or more hydroxy or halogen groups.

Particular compounds of formula VII are such that: 1) if X is O, R₁ is alkyl of 1 to 4 carbons, phenyl, benzyl or phenylethyl, and R₂ is hydrogen, at least two of R₆, R₇, R₈ and R₉ are not hydroxyl or acetate; 2) if X is O, R₁ is methyl, R₂ is hydrogen, R₆ and R₇ are both hydroxyl, and one of R₈ and R₉ is hydrogen, the other is not NHC(O)R_{9B}; 3) if X is O, R₁ is OR_{1A}, R_{1A} is hydrogen or lower alkyl, and R₂ is hydrogen, at least one, but not all, of R₆, R₇, R₈ and R₉ is hydroxyl or acetate.

In particular embodiments, biologically active THI derivatives according to Formula VII are compounds according to Formula VII(a):

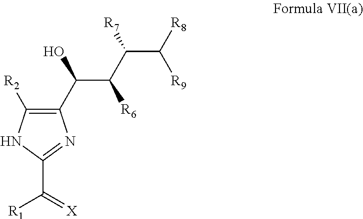

In other embodiments, biologically active THI derivatives are compounds according to Formula VIII:

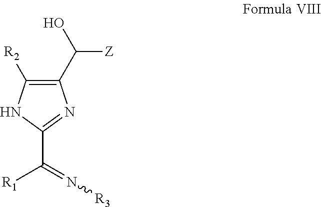

including any pharmaceutically acceptable salts, esters, isomers or solvates (e.g., hydrates) thereof, wherein: Z is optionally substituted alkyl; R₁ is OR_{1A}, NHOH, hydrogen, or optionally substituted alkyl, aryl, alkylaryl, arylalkyl, heteroalkyl, heterocycle, alkylheterocycle, or heterocyclealkyl; R₂ is OR_{2A}, C(O)OR_{2A}, hydrogen, halogen, nitrile, or optionally substituted alkyl, aryl, alkylaryl, arylalkyl, heteroalkyl, heterocycle, alkylheterocycle, or heterocyclealkyl; R₃ is OR_{3A}, N(R_{3A})₂, NHC(O)R_{3A}, NHSO₂R_{3A}, or hydrogen; and each of R_{1A}, R_{2A}, and R_{3A} is independently hydrogen or optionally substituted alkyl, aryl, alkylaryl, arylalkyl, heteroalkyl, heterocycle, alkylheterocycle, or heterocyclealkyl.

In particular embodiments, biologically active THI derivatives according to Formula VIII are compounds according to Formula VIII(a):

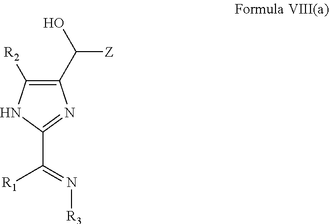

In other embodiments, biologically active THI derivatives are compounds according to Formula IX:

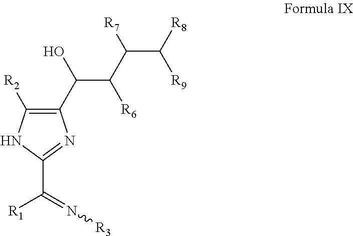

including any pharmaceutically acceptable salts, esters, isomers or solvates (e.g., hydrates) thereof, wherein: R₁ is OR_{1A}, NHOH, hydrogen, or optionally substituted alkyl, aryl, alkylaryl, arylalkyl, heteroalkyl, heterocycle, alkylheterocycle, or heterocyclealkyl; R₃ is OR_{3A}, N(R_{3A})₂, NHC(O)R_{3A}, NHSO₂R_{3A}, or hydrogen; R₆ is OR_{6A}, OC(O)R_{6A}, N(R_{6B})₂, NHC(O)R_{6B}, hydrogen, or halogen; R₇ is OR_{7A}, OC(O)R_{7A}, N(R_{7B})₂, NHC(O)R_{7B}, hydrogen, or halogen; R₈ is OR_{8A}, OC(O)R_{8A}, N(R_{8B})₂, NHC(O)R_{8B}, hydrogen, or halogen; R₉ is CH₂OR_{9A}, CH₂OC(O)R_{9A}, N(R_{9B})₂, NHC(O)R_{9B}, hydrogen, or halogen; and each of R_{1A}, R_{3A}, R_{6A}, R_{7A}, R_{8A} and R_{9A} is independently hydrogen or optionally substituted alkyl, aryl, alkylaryl, arylalkyl, heteroalkyl, heterocycle, alkylheterocycle, or heterocyclealkyl.

In particular embodiments, biologically active THI derivatives according to Formula IX are compounds according to Formula IX(a) or IX(b):

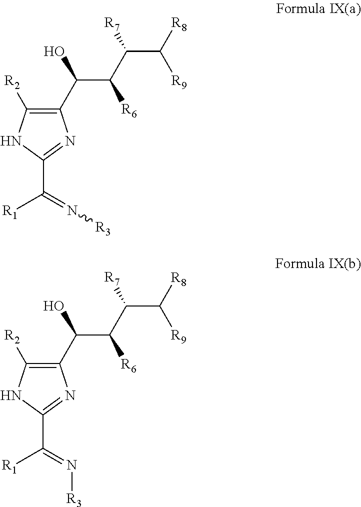

In specific embodiments, the biologically active THI derivatives are selected from the following:
(1R,2S,3R)-1-(2-(5-methylisoxazol-3-yl)-1H-imidazol-5-yl)butane-1,2,3,4-tetraol;
(1R,2S,3R)-1-(2-(5-ethylisoxazol-3-yl)-1H-imidazol-5-yl)butane-1,2,3,4-tetraol;
(1R,2S,3R)-1-(2-(isoxazol-3-yl)-1H-imidazol-5-yl)butane-1,2,3,4-tetraol;
(1R,2S,3R)-1-(2-(isoxazol-3-yl)-1H-imidazol-4-yl)butane-1,2,3,4-tetraol;
(1R,2S,3R)-1-(2-(2-methylthiazol-4-yl)-1H-imidazol-4-yl)butane-1,2,3,4-tetraol;
(1R,2S,3R)-1-(2-(1-benzyl-1H-1,2,4-triazol-3-yl)-1H-imidazol-4-yl)butane-1,2,3,4-tetraol hydrochloride;
(1R,2S,3R)-1-(1H,1′H-2,2′-biimidazol-5-yl)butane-1,2,3,4-tetraol;
(1R,2S,3R)-1-(2-(5-methoxy-4,5-dihydroisoxazol-3-yl)-1H-imidazol-5-yl)butane-1,2,3,4-tetraol;
(1R,2S,3R)-1-(2-(5-methyl-1H-pyrazol-3-yl)-1H-imidazol-5-yl)butane-1,2,3,4-tetraol;
1-(4-((1R,2S,3R)-1,2,3,4-tetrahydroxybutyl)-1H-imidazol-2-yl)-ethanone oxime;
(E)-1-(4-((1R,2S,3R)-1,2,3,4-tetrahydroxybutyl)-1H-imidazol-2-yl)-ethanone oxime;
(Z)-1-(4-((1R,2S,3R)-1,2,3,4-tetrahydroxybutyl)-1H-imidazol-2-yl)-ethanone oxime;
1-(4-((1R,2S,3R)-1,2,3,4-tetrahydroxybutyl)-1H-imidazol-2-yl)ethanone O-methyl oxime; (E)-1-(4-((1R,2S,3R)-1,2,3,4-tetrahydroxybutyl)-1H-imidazol-2-yl)ethanone O-methyl oxime;
(Z)-1-(4-((1R,2S,3R)-1,2,3,4-tetrahydroxybutyl)-1H-imidazol-2-yl)ethanone O-methyl oxime;
N′-(1-(4-((1R,2S,3R)-1,2,3,4-tetrahydroxybutyl)-1H-imidazol-2-yl)ethylidene)acetohydrazide;
4-methyl-N′-(1-(4-((1R,2S,3R)-1,2,3,4-tetrahydroxybutyl)-1H-imidazol-2-yl)ethylidene)benzenesulfonohydrazide;
(E)-4-methyl-N′-(1-(4-((1R,2S,3R)-1,2,3,4-tetrahydroxybutyl)-1H-imidazol-2-yl)ethylidene)benzenesulfonohydrazide;
(Z)-4-methyl-N′-(1-(4-((1R,2S,3R)-1,2,3,4-tetrahydroxybutyl)-1H-imidazol-2-yl)ethylidene)benzenesulfonohydrazide;
N′-(1-(4-((1R,2S,3R)-1,2,3,4-tetrahydroxybutyl)-1H-imidazol-2-yl)ethylidene)benzohydrazide;
Ethyl2-(1-(4-((1R,2S,3R)-1,2,3,4-tetrahydroxybutyl)-1H-imidazol-2-yl)ethylidene)hydrazinecarboxylate;
(E)-ethyl2-(1-(4-((1R,2S,3R)-1,2,3,4-tetrahydroxybutyl)-1H-imidazol-2-yl)ethylidene)hydrazinecarboxylate;
(Z)-ethyl2-(1-(4-((1R,2S,3R)-1,2,3,4-tetrahydroxybutyl)-1H-imidazol-2-yl)ethylidene)hydrazinecarboxylate;
N′-(1-(4-((1R,2S,3R)-1,2,3,4-tetrahydroxybutyl)-1H-imidazol-2-yl)ethylidene)nicotinohydrazide;
(E)-N′-(1-(4-((1R,2S,3R)-1,2,3,4-tetrahydroxybutyl)-1H-imidazol-2-yl)ethylidene)nicotinohydrazide;
(Z)—N′-(1-(4-((1R,2S,3R)-1,2,3,4-tetrahydroxybutyl)-1H-imidazol-2-yl)ethylidene)nicotinohydrazide;
3-chloro-N′-(1-(4-((1R,2S,3R)-1,2,3,4-tetrahydroxybutyl)-1H-imidazol-2-yl)ethylidene)benzohydrazide;
4-fluoro-N′-(1-(4-((1R,2S,3R)-1,2,3,4-tetrahydroxybutyl)-1H-imidazol-2-yl)ethylidene)benzohydrazide;
(E)-4-fluoro-N′-(1-(4-((1R,2S,3R)-1,2,3,4-tetrahydroxybutyl)-1H-imidazol-2-yl)ethylidene)benzohydrazide;
(Z)-4-fluoro-N′-(1-(4-((1R,2S,3R)-1,2,3,4-tetrahydroxybutyl)-1H-imidazol-2-yl)ethylidene)benzohydrazide;
6-amino-N′-(1-(4-((1R,2S,3R)-1,2,3,4-tetrahydroxybutyl)-1H-imidazol-2-yl)ethylidene)nicotinohydrazide;
(E)-6-amino-N′(1-(4-((1R,2S,3R)-1,2,3,4-tetrahydroxybutyl)-1H-imidazol-2-yl)ethylidene)nicotinohydrazide;
(Z)-6-amino-N′-(1-(4-((1R,2S,3R)-1,2,3,4-tetrahydroxybutyl)-1H-imidazol-2-yl)ethylidene)nicotinohydrazide;
N′-(1-(4-((1R,2S,3R)-1,2,3,4-tetrahydroxybutyl)-1H-imidazol-2-yl)ethylidene)isonicotinohydrazide;
(E)-N′-(1-(4-((1R,2S,3R)-1,2,3,4-tetrahydroxybutyl)-1H-imidazol-2-yl)ethylidene)isonicotinohydrazide;
(Z)—N′-(1-(4-((1R,2S,3R)-1,2,3,4-tetrahydroxybutyl)-1H-imidazol-2-yl)ethylidene)isonicotinohydrazide;
N′-(1-(4-((1R,2S,3R)-1,2,3,4-tetrahydroxybutyl)-1H-imidazol-2-yl)ethylidene)biphenyl-3-carbohydrazide;
(E)-N′-(1-(4-((1R,2S,3R)-1,2,3,4-tetrahydroxybutyl)-1H-imidazol-2-yl)ethylidene)biphenyl-3-carbohydrazide; and
(Z)—N′-(1-(4-((1R,2S,3R)-1,2,3,4-tetrahydroxybutyl)-1H-imidazol-2-yl)ethylidene)biphenyl-3-carbohydrazide.

In further embodiments of the S1P promoting compositions described herein, a sphingosine analog may be included as an S1P promoting agent. A sphingosine analog as described herein may be phosphorylated in the cell and act as a S1P receptor agonist. Examples of sphingosine analogs and S1P receptor agonists are described, for example, in U.S. Patent Application Publication No. 2011/0306672 and U.S. Patent Application Publication No. 2011/0229501, each of which are incorporated by reference herein. Where included in the compositions described herein, a sphingosine analog, such as, for example, FTY720 (2-amino-2-[2-(4-octylphenyl)ethyl]propane-1,3-diol), may be provided as a compound according to Formula X:

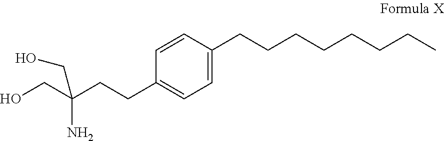 [SMILES: CCCCCCCCc1ccc(CCC(N)(CO)CO)cc1]

including any pharmaceutically acceptable salts, esters, isomers or solvates (e.g., hydrates) thereof.

The SPL inhibitors described herein, including THI and the biologically active THI derivatives described herein may contain one or more stereocenters, and can exist as racemic mixtures of enantiomers or mixtures of diastereomers. The SPL inhibitors described herein can be provided as stereomerically pure forms of such compounds, as well as mixtures of enantiomers or mixtures of diastereomers. Stereoisomers may be asymmetrically synthesized or resolved using standard techniques such as chiral columns or chiral resolving agents. See, e.g., Jacques, J., et al, Enantiomers, Racemates and Resolutions (Wiley Interscience, New York, 1981); Wilen, S. H., et al., Tetrahedron 33:2725 (1977); Eliel, E. L., Stereochemistry of Carbon Compounds (McGraw Hill, N.Y., 1962); and Wilen, S. H., Tables of Resolving Agents and Optical Resolutions, p. 268 (E. L. Eliel, Ed., Univ. of Notre Dame Press, Notre Dame, Ind., 1972). The current disclosure further encompasses configurational isomers of compounds disclosed herein, either in admixture or in pure or substantially pure form, such as cis (Z) and trans (E) alkene isomers and syn and anti oxime isomers.

The SPL inhibitors described herein can be prepared by methods known in the art (e.g., by varying and adding to the approaches described in Pyne, S. G., ACGC Chem. Res. Comm. 11:108-112 (2000); Halweg, K. M. and Büchi, G., J. Org. Chem. 50:1134-1136 (1985), and by approaches described, for example, in U.S. Pat. No. 7,825,150 and U.S. Pat. No. 7,598,280).

The S1P promoting compositions described herein are prepared to facilitate administration of therapeutically effective amounts of one or more S1P promoting agents to a subject. For purposes of the present disclosure, a therapeutically effective amount of an S1P promoting agent is an amount sufficient to treat a degenerative muscle condition. In particular embodiments, a therapeutically effective amount of an S1P promoting agent as described herein is an amount sufficient to result in one or more of the following in a subject: promotion of increase local or systemic levels of S1P; inhibition of degeneration of muscle tissue; promotion of muscle tissue regeneration; promotion of satellite cell proliferation in muscle tissue; inhibition of fat deposition in muscle tissue; inhibition of fibrosis in muscle tissue; and promotion of an increase in muscle fiber size or number. In some embodiments, the S1P promoting compositions may include a single S1P promoting agent selected from S1P, THI, and the biologically active THI derivatives described herein. In other embodiments, the S1P promoting compositions may include two or more S1P promoting agents selected from S1P, THI, and the biologically active THI derivatives described herein.

The S1P promoting compositions described herein may be may be prepared using pharmaceutically acceptable carriers, such as those described, for example, in Remington's Pharmaceutical Sciences, 18th ed., Mack Publishing, Easton Pa. (1990). Additionally, S1P promoting compositions may be formulated and prepared for delivery to a subject via any suitable route of administration. For example, S1P promoting compositions as described herein may be prepared as formulations and dosage forms suitable for oral, mucosal (e.g., nasal, sublingual, vaginal, buccal, or rectal), parenteral (e.g., subcutaneous, intravenous, bolus injection, intramuscular, or intraarterial), pulmonary, or transdermal administration. Examples of suitable dosage forms include, but are not limited to, the following: tablets; caplets; capsules, such as soft elastic gelatin capsules; sachets; troches; lozenges; dispersions; suppositories; ointments; poultices; pastes; powders; dressings; creams; plasters; solutions; patches; aerosols (e.g., nasal sprays or inhalers); gels; liquid dosage forms suitable for oral or mucosal administration to a subject, including suspensions (e.g., aqueous or non-aqueous liquid suspensions, oil-in-water emulsions, or a water-in-oil liquid emulsions), solutions, and elixirs; liquid dosage forms suitable for parenteral administration to a subject; and sterile solids (e.g., crystalline or amorphous solids) that can be administered or reconstituted prior to administration for oral, parenteral, or pulmonary administration to a subject. The composition and type of dosage form will vary depending on, at least in part, the desired therapeutic effect, route of administration, and degenerative muscle condition to be treated.

The S1P promoting compositions described herein may be formulated to suit the desired route of administration. For example, compositions prepared for oral administration may require one or more coatings or carriers that facilitate one or more of the following: delivery of an S1P promoting agent to the subject; preservation of the physical or chemical stability of the dosage form or S1P promoting agent in the gastrointestinal environment; controlled release of an S1P promoting agent; uptake of an S1P promoting agent in the subject's circulatory system; and delivery of an S1P promoting agent across cell membranes to intracellular sites.

Poorly soluble compounds often present delivery challenges, as they can be difficult to administer to a subject in a manner that provides desirable bioavailability. In certain instances, where the S1P promoting agents described herein are poorly soluble compounds, the S1P promoting compositions may be prepared as liquid dosage forms (or dosage forms suitable for reconstitution) that facilitate solubilization of the S1P promoting agent with the aid of, for example, solubilizing agents, emulsifiers and surfactants such as, but not limited to, cyclodextrins (e.g., α-cyclodextrin, β-cyclodextrin, Captisol™, and Encapsin™ (see, e.g., Davis and Brewster, 2004, Nat. Rev. Drug Disc. 3:1023-1034), Labrasol®, Labrafil®, Labrafac®, cremafor, and non-aqueous solvents, such as, but not limited to, ethyl alcohol, isopropyl alcohol, ethyl carbonate, ethyl acetate, benzyl alcohol, benzyl benzoate, propylene glycol, 1,3-butylene glycol, dimethyl formamide, dimethyl sulfoxide (DMSO), biocompatible oils (e.g., cottonseed, groundnut, corn, germ, olive, castor, and sesame oils), glycerol, tetrahydrofurfuryl alcohol, polyethylene glycols, fatty acid esters of sorbitan, and mixtures thereof (e.g., DMSO:cornoil). Alternatively, S1P promoting agents exhibiting poor solubility may be processed, such as by known micronization techniques, into mico- or nanoparticulate material. In doing so, effective solubility and bioavailability may be increased. S1P promoting agents prepared as micro- or nanoparticulate materials may be formulated into any suitable suspension, powder, or granulation that is readily administered to a subject.

Where prepared as oral dosage forms, the S1P promoting compositions described herein may be prepared as, for example, tablets, caplets, capsules, and liquids (e.g., syrups, solutions, and suspensions). Such dosage forms may contain predetermined amounts of active ingredients, and may be prepared by methods of pharmacy well known to those skilled in the art. See, e.g., Remington's Pharmaceutical Sciences, 18th ed., Mack Publishing, Easton Pa. (1990). Dosage forms for oral delivery may be prepared by combining an S1P promoting agent in an intimate admixture with at least one pharmaceutically acceptable carrier according to conventional pharmaceutical compounding techniques. The pharmaceutically acceptable carrier may be selected and formulated to suit a particular agent or dosage form, and can include, for example, pharmaceutically acceptable, carriers, excipients, surfactants, permeation enhancers, solvents, adjuvants, disintegrants, and lubricants.

Where prepared for parenteral delivery, S1P promoting compositions according to the present description can be formulated for administration by various routes including, but not limited to, subcutaneous, intravenous (including bolus injection), intramuscular, and intraarterial injection or infusion. Because parenteral routes of administration typically bypasses subjects' natural defenses, parenteral dosage forms are prepared as sterile or capable of being sterilized prior to administration. Examples of parenteral dosage forms include, but are not limited to, solutions ready for injection, dry products ready to be reconstituted, dissolved or suspended in a pharmaceutically acceptable vehicle for injection, suspensions ready for injection, and emulsions. Examples of suitable carriers or vehicles that can be used to provide parenteral dosage forms are well known and include, but are not limited to, the following: Water for Injection USP; aqueous vehicles such as sodium chloride solution, Ringer's solution, dextrose solution, dextrose and sodium chloride solution, and lactated Ringer's solution for injection; water-miscible vehicles such as ethyl alcohol, polyethylene glycol, and polypropylene glycol; and non-aqueous vehicles such as suitable vegetable and plant oils, ethyl oleate, isopropyl myristate, and benzyl benzoate.

S1P promoting compositions prepared as transdermal, topical, and mucosal dosage forms include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Transdermal dosage forms incorporating an S1P promoting composition may be prepared as “reservoir type” or “matrix type” patches, which can be applied to the skin and worn for a specific period of time to permit the penetration of a desired amount of S1P promoting agent. Examples of suitable carriers, diluents, and other materials that can be used to provide transdermal, topical, and mucosal dosage forms are well known to those skilled in the pharmaceutical arts, and depend on the particular tissue to which a given pharmaceutical composition or dosage form will be applied. Where the S1P promoting composition is prepared for transdermal delivery of an S1P promoting agent, one or more permeation enhancers may be included in, used in conjunction with, or used subsequent to treatment with the S1P promoting composition.

### III. METHODS OF USE

The S1P promoting compositions described herein are useful for treating a subject suffering from a degenerative muscle condition. In particular embodiments, administration of the S1P promoting composition results in an increase in S1P of at least about 5%, 10%, 15%, 20%, 25%, 30%, 35%, 40%, 45%, 50%, 60%, 70%, 80%, 90%, 100%, 110%, 120%, 130%, 140%, 150%, 160%, 170%, 180%, 190%, and 200%, or more, relative to controls. In further embodiments, the administration of an S1P promoting composition results in increases of S1P levels by an amount ranging from at least about a 2-fold increase to at least about a 20-fold increase. In such further embodiments, administration of an S1P promoting composition results in increases of S1P levels of at least about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, and 20-fold, or more, relative to controls.

In particular embodiments, methods for inhibiting muscle degeneration associated with a degenerative muscle condition are provided, wherein the method includes administering a therapeutically effective amount of an S1P promoting composition to a subject suffering from the degenerative muscle condition. In such embodiments, the degenerative muscle condition may be selected from muscle injury, including acute muscle injury, resulting in loss of muscle tissue, muscle atrophy, wasting or degeneration, muscle overuse, muscle disuse atrophy, sarcopenia and muscular dystrophy. In one such embodiment, the degenerative muscle condition is selected from DMD and BMD. In another such embodiment, the degenerative muscle condition is selected from muscle injury, including acute muscle injury, resulting in loss of muscle tissue, muscle atrophy, wasting or degeneration, muscle overuse, and muscle disuse atrophy. In yet another embodiment, the degenerative muscle condition is sarcopenia. In specific embodiments, administration of the S1P promoting composition results in one or more of the following: an increase in S1P, satellite cell proliferation, an increase in muscle fiber size or number, a decrease in fibrosis, and a decrease in fat deposition.

Methods for promoting proliferation of satellite cells in muscle tissue of a subject suffering from a degenerative muscle condition are also provided. Such methods include administering a therapeutically effective amount of an S1P promoting composition to a subject suffering from the degenerative muscle condition. In embodiments of such methods, the degenerative muscle condition may be selected from muscle injury, including acute muscle injury, resulting in loss of muscle tissue, muscle atrophy, wasting or degeneration, muscle overuse, muscle disuse atrophy, sarcopenia and muscular dystrophy. In one such embodiment, the degenerative muscle condition is selected from DMD and BMD. In another such embodiment, the degenerative muscle condition is selected from muscle injury, including acute muscle injury, resulting in loss of muscle tissue, muscle atrophy, wasting or degeneration, muscle overuse, and muscle disuse atrophy. In yet another embodiment, the degenerative muscle condition is sarcopenia. In particular embodiments of such methods, administering the therapeutically effective amount of the S1P promoting composition promotes proliferation of satellite cells resulting in increases, measured as a total cross-sectional area of a muscle tissue, of at least about 5% higher to at least about 100% higher, relative to controls. In certain such embodiments of the methods disclosed herein, administering a therapeutically effective amount of an S1P promoting composition increases the proliferation of satellite cells by at least about 5%, 10%, 15%, 20%, 25%, 30%, 35%, 40%, 45%, 50%, 60%, 70%, 80%, 90%, and 100%, or more, relative to controls. In further embodiments, the administration of an S1P promoting composition results in an increased proliferation of satellite cells by an amount ranging from at least about a 1.5-fold increase to at least about a 10-fold increase, or more. In still further embodiments, administration of an S1P promoting composition results in an increased proliferation of satellite cells of at least about 1.5-fold, 2-fold, 3-fold, 4-fold, 5-fold, 6-fold, 7-fold, 8-fold, 9-fold, and 10-fold, or more.

In other embodiments of the methods disclosed herein, administering a therapeutically effective amount of the S1P promoting composition promotes the proliferation of satellite cells in the muscle tissue, wherein the proliferation of satellite cells in muscle tissue is measured by detecting embryonic myosin heavy chain expression. In certain such embodiments, the proliferation of satellite cells in the muscle tissue is indicated by an increase of embryonic myosin heavy chain expression of at least about 10% higher to at least about 300% higher, relative to controls. In particular such embodiments, the proliferation of satellite cells in muscle tissue is indicated by an increase of embryonic myosin heavy chain expression of at least about 10%, 20%, 30%, 40%, 50%, 60%, 70%, 80%, 90%, 100% 125%, 150%, 175%, 200%, 225%, and 250%, or more, relative to controls. In still other embodiments, the proliferation of satellite cells in muscle tissue is indicated by an increase of embryonic myosin heavy chain expression of at least about 2-fold higher, 3-fold higher, 4-fold higher, and 5-fold higher, or more, relative to controls.

Methods for inhibiting fat deposition in muscle tissue of a subject suffering from a degenerative muscle condition are also provided. Such methods include administering a therapeutically effective amount of an S1P promoting composition to a subject suffering from the degenerative muscle condition. In embodiments of such methods, the degenerative muscle condition may be selected from muscle injury, including acute muscle injury, resulting in loss of muscle tissue, muscle atrophy, wasting or degeneration, muscle overuse, muscle disuse atrophy, sarcopenia and muscular dystrophy. In one such embodiment, the degenerative muscle condition is selected from DMD and BMD. In another such embodiment, the degenerative muscle condition is selected from muscle injury, including acute muscle injury, resulting in loss of muscle tissue, muscle atrophy, wasting or degeneration, muscle overuse, and muscle disuse atrophy. In yet another embodiment, the degenerative muscle condition is sarcopenia. Administering the therapeutically effective amount of the S1P promoting composition inhibits fat deposition in the muscle tissue of the subject, and in certain embodiments, fat deposition is reduced in an amount ranging from at least about 10% to at least about 75% when compared to controls. In particular embodiments of the methods for inhibiting fat deposition disclosed herein, fat deposition is reduced by at least about 10%, 15%, 20%, 25%, 30%, 35%, 40%, 45%, 50%, 55%, 60%, 65%, 70%, and 75%, or more, relative to controls. In further embodiments of such methods, administering a therapeutically effective amount of an S1P promoting composition inhibits fat deposition in the heart and/or diaphragm of a subject.

Methods for inhibiting fibrosis in muscle tissue of a subject suffering from a degenerative muscle condition are also provided. Such methods include administering a therapeutically effective amount of an S1P promoting composition to a subject suffering from the degenerative muscle condition. In embodiments of such methods, the degenerative muscle condition may be selected from muscle injury, including acute muscle injury, resulting in loss of muscle tissue, muscle atrophy, wasting or degeneration, muscle overuse, muscle disuse atrophy, sarcopenia and muscular dystrophy. In one such embodiment, the degenerative muscle condition is selected from DMD and BMD. In another such embodiment, the degenerative muscle condition is selected from muscle injury, including acute muscle injury, resulting in loss of muscle tissue, muscle atrophy, wasting or degeneration, muscle overuse, and muscle disuse atrophy. In yet another embodiment, the degenerative muscle condition is sarcopenia. Administering the therapeutically effective amount of the S1P promoting composition inhibits fibrosis in the muscle tissue of the subject, and in certain embodiments, fibrosis is reduced by at least about 2% to at least about 50% lower than controls. In particular embodiments of the methods for inhibiting fibrosis in muscle tissue disclosed herein, fibrosis in muscle tissue of a subject suffering from a degenerative muscle condition is reduced by at least about 2%, 5%, 7%, 10%, 20%, 35%, 40%, 45%, and 50% or more, relative to controls. In further embodiments, administering a therapeutically effective amount of an S1P promoting composition inhibits muscle fibrosis in the heart and/or diaphragm of a subject.

In further embodiments, methods for promoting an increase in muscle fiber size in muscle tissue of a subject suffering from a degenerative muscle condition are also provided. Such methods include administering a therapeutically effective amount of an S1P promoting composition to a subject suffering from the degenerative muscle condition. In embodiments of such methods, the degenerative muscle condition may be selected from muscle injury, including acute muscle injury, resulting in loss of muscle tissue, muscle atrophy, wasting or degeneration, muscle overuse, muscle disuse atrophy, sarcopenia and muscular dystrophy. In one such embodiment, the degenerative muscle condition is selected from DMD and BMD. In another such embodiment, the degenerative muscle condition is selected from muscle injury, including acute muscle injury, resulting in loss of muscle tissue, muscle atrophy, wasting or degeneration, muscle overuse, and muscle disuse atrophy. In yet another embodiment, the degenerative muscle condition is sarcopenia. Administering the therapeutically effective amount of the S1P promoting composition promotes an increase in muscle fiber size in muscle tissue of the subject, and in certain embodiments, muscle fiber size is increased by an amount ranging from at least about a 5% increase to at least about a 50% increase over controls. In particular embodiments of the methods disclosed herein for promoting an increase in muscle fiber size, the muscle fiber size in the subject is increased by about 5%, 7%, 10%, 15%, 20%, 25%, 30%, 35%, 40%, 45%, and 50% or more, relative to controls. An increase in muscle fiber size may include an increase in muscle fiber diameter, volume, or length. In further embodiments, administering a therapeutically effective amount of an S1P promoting composition increases muscle fiber size in the heart and/or diaphragm of a subject.

In still further embodiments, methods for promoting regeneration of muscle tissue in a subject suffering from a degenerative muscle condition or other muscle injury or atrophy are provided. Such methods include administering a therapeutically effective amount of an S1P promoting composition to a subject suffering from the degenerative muscle condition. In embodiments of such methods, the degenerative muscle condition may be selected from muscle injury, including acute muscle injury, resulting in loss of muscle tissue, muscle atrophy, wasting or degeneration, muscle overuse, muscle disuse atrophy, sarcopenia and muscular dystrophy. In one such embodiment, the degenerative muscle condition is selected from DMD and BMD. In another such embodiment, the degenerative muscle condition is selected from muscle injury, including acute muscle injury, resulting in loss of muscle tissue, muscle atrophy, wasting or degeneration, muscle overuse, and muscle disuse atrophy. In yet another embodiment, the degenerative muscle condition is sarcopenia. In still other such embodiments, the degenerative muscle condition may be associated, dysferlinopathy, AIDS/HIV, diabetes, chronic obstructive pulmonary disease, kidney disease, cancer, aging, autoimmune disease, polymyositis, and dermatomyositis.

The S1P promoting composition administered in each of the embodiments of the methods of use described herein is an S1P promoting composition according to the present description. In some embodiments of the methods of use described herein, the S1P promoting composition administered to the subject includes S1P as an S1P promoting agent. In other embodiments of the methods of use described herein, the S1P promoting composition administered to the subject includes THI as an S1P promoting agent. In still other embodiments of the methods of use described herein, the S1P promoting composition administered to the subject includes a biologically active THI derivative as disclosed herein as an S1P promoting agent. In yet other embodiments of the methods of use described herein, the S1P promoting composition administered to the subject includes a combination of two or more S1P promoting agents selected from S1P, THI, and biologically active THI derivatives as disclosed herein. In further embodiments of the methods of use described herein, the S1P promoting composition administered to the subject includes one or more S1P promoting agents selected from S1P, sphingosine analogs, S1P receptor agonists, THI, and THI derivatives, in combination with other compounds or drugs that increase S1P, and/or other treatments for muscle degeneration.

In embodiments of the methods of use described herein, a therapeutically effective amount of the S1P promoting agents described herein may be from about 0.001 mg/kg to about 100 mg/kg body weight per day. For example, S1P promoting compositions according to the present description can be prepared and administered such that the amount of an S1P promoting agent according to the present description administered to a subject is selected from between about 0.001 mg/kg and about 50 mg/kg, between about 0.01 mg/kg and about 20 mg/kg, between about 0.1 and about 10 mg/kg, and between about 0.1 mg/kg and about 5 mg/kg body weight per day.

In particular embodiments of the methods of use described herein, an S1P promoting composition as disclosed herein may be administered orally (PO) or intravenously (IV) at a frequency of daily, twice-daily, three-times daily, or more or less frequently.

In some embodiments of the methods of use described herein, the S1P promoting compositions may be delivered or administered locally to the area in need of treatment. Local administration can be achieved by, for example, and not by way of limitation, local injection into affected muscle, by means of a catheter, by means of a suppository, or by means of an implant. In one embodiment, administration can be by direct injection at the site (or former site) of a malignant tumor or neoplastic or pre-neoplastic tissue. In another embodiment, the S1P promoting compositions described herein can be delivered in a vesicle, in particular a liposome (see, e.g., Langer, Science 249:1527-33, 1990; Treat et al, In Liposomes in the Therapy of Infectious Disease and Cancer, Lopez-Berestein and Fidler (eds.), Liss, New York, pp. 353-65, 1989; Lopez-Berestein, supra, pp. 317-27).

In yet other embodiments of the methods of use, S1P promoting compositions can be delivered in a controlled release system. In one such embodiment, a pump can be used (see, e.g., Langer, supra; Sefton, CRC Crit. Ref. Biomed. Eng. 14:201, 1987; Buchwald et al., Surgery 88:507, 1980; Saudek et al., N. Engl. J. Med. 321:574, 1989). In another such embodiment, a polymeric controlled release system or formulation can be used (see, e.g., Medical Applications of Controlled Release, Langer and Wise (eds.), CRC Pres., Boca Raton, Fla., 1974; Controlled Drug Bioavailability, Drug Product Design and Performance, Smolen and Ball (eds.), Wiley, New York, 1984; Ranger and Peppas, J. Macro mol. Sci. Rev. Macromol. Chem. 23:61, 1983; see also Levy et al, Science 228: 190, 1985; During et al, Ann. Neurol. 25:351, 1989; Howard et al, J. Neurosurg. 71:105, 1989). In yet another such embodiment, a controlled release system delivering the S1P promoting composition can be placed in proximity of the therapeutic target, thus requiring a reduced systemic dose (see, e.g., Goodson, Medical Applications of Controlled Release, supra, Vol. 2, pp. 115-138, 1984). Other controlled release systems are discussed in, for example, the review by Langer (Science 249: 1527-1533, 1990).

However, a specific dosage and treatment regime for any particular subject or disease state will depend upon a variety of factors, including the age, body weight, general health, sex, diet, time of administration, nature of active compound(s), rate of excretion, drug combination, the judgment of the treating physician, and the severity of the particular disease being treated. Moreover, determination of the amount of a pharmaceutical composition to be administered to a subject will depend upon, among other factors, the amount and specific activity of the S1P promoting agent(s) included in the S1P promoting composition and the use or incorporation of additional therapeutic or prophylactic agents or treatment regimes. Determination of therapeutically effective dosages may be based on animal model studies and is typically guided by determining effective dosages and administration protocols that significantly reduce the occurrence or severity of the apoptosis-associated disease in model subjects.

The following examples are provided merely as illustrative of various aspects of the invention and shall not be construed to limit the invention. It is to be understood that the disclosed compositions and methods are not limited to the particular methodologies, protocols, and reagents described herein. In each instance, unless otherwise specified, standard materials and methods were used in carrying out the work described in the Examples provided. All patent and literature references cited herein are hereby incorporated by reference in their entirety.

### EXAMPLES

### Example 1

### Suppression of Dystrophic Muscle and Activity Phenotypes in Drosophila by Reducing Wunen, a Lipid Phosphate Phosphatase

Among the phenotypes observed in Dystrophin mutant flies is a visible wing vein defect which was used to identify wunen, a lipid phosphate phosphatase (LPP), which suppresses the Dystrophin mutation. The protein wunen is the fly homolog of mammalian LPP3. LPPs have a broad substrate range and act upon phosphatidic acid (PA), sphingosine 1-phosphate (S1P) and their derivatives. LPP3 has been shown to specifically attenuate levels of S1P in human cells indicating a preference for this substrate. S1P has been implicated in satellite cell proliferation and myoblast differentiation in vitro. As such, it was hypothesized that reduced wunen may suppress dystrophic phenotypes in Drosophila.

A molecular phenotype in Drosophila at the myofibril structural level was used to assess Dystrophin muscle mutants. Two titin-like proteins, Projectin and Kettin form the connecting filaments in indirect flight muscles (IFMs) of Drosophila that link the Z-bands to thick filaments and function in muscle elasticity during oscillatory work in flight.

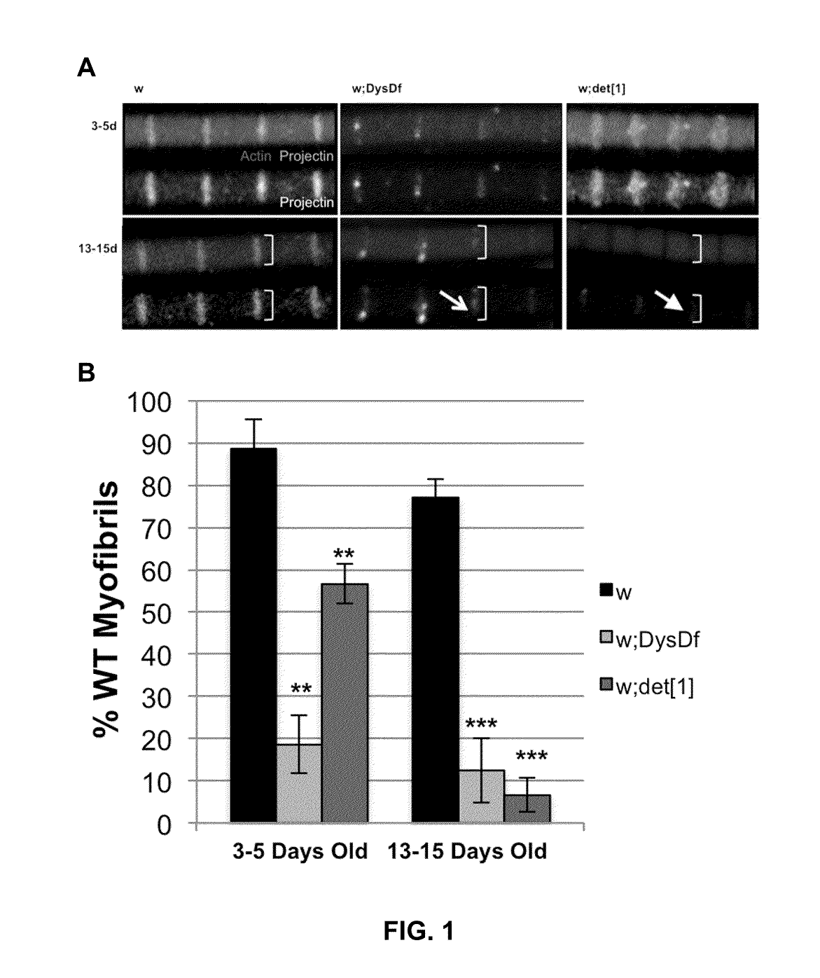

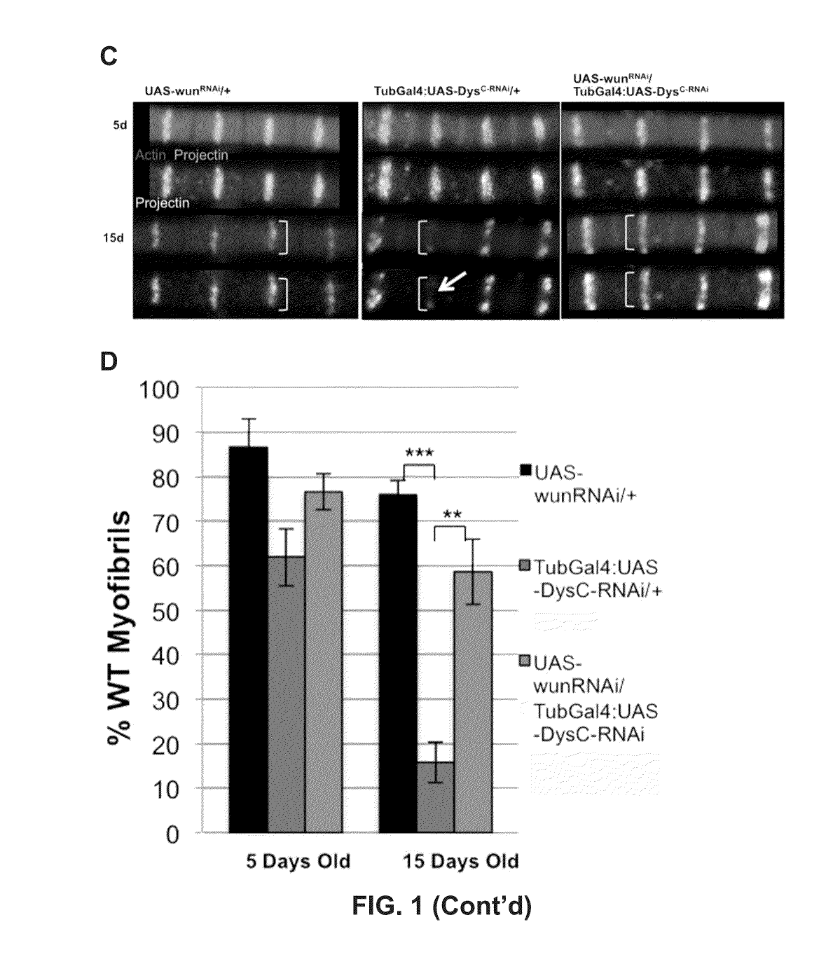

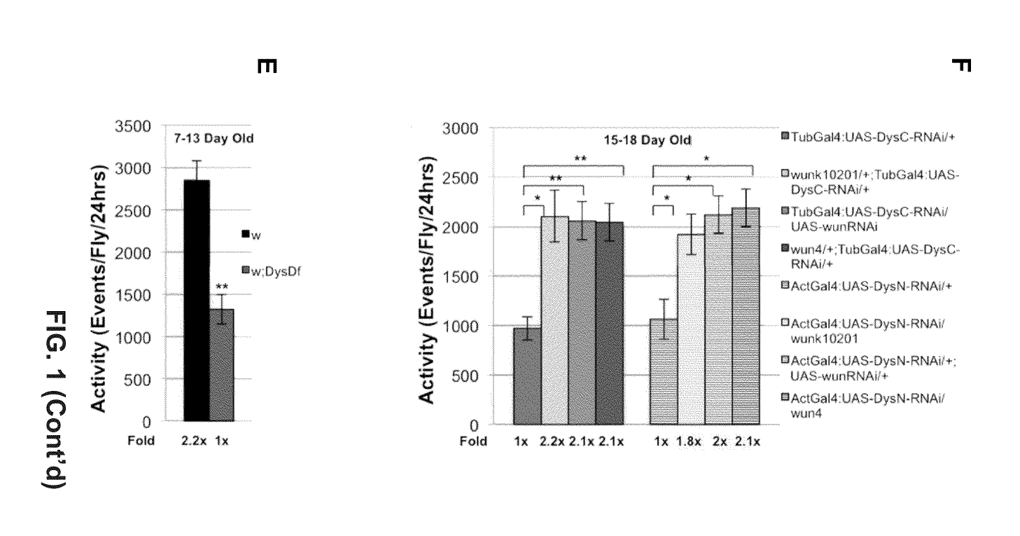

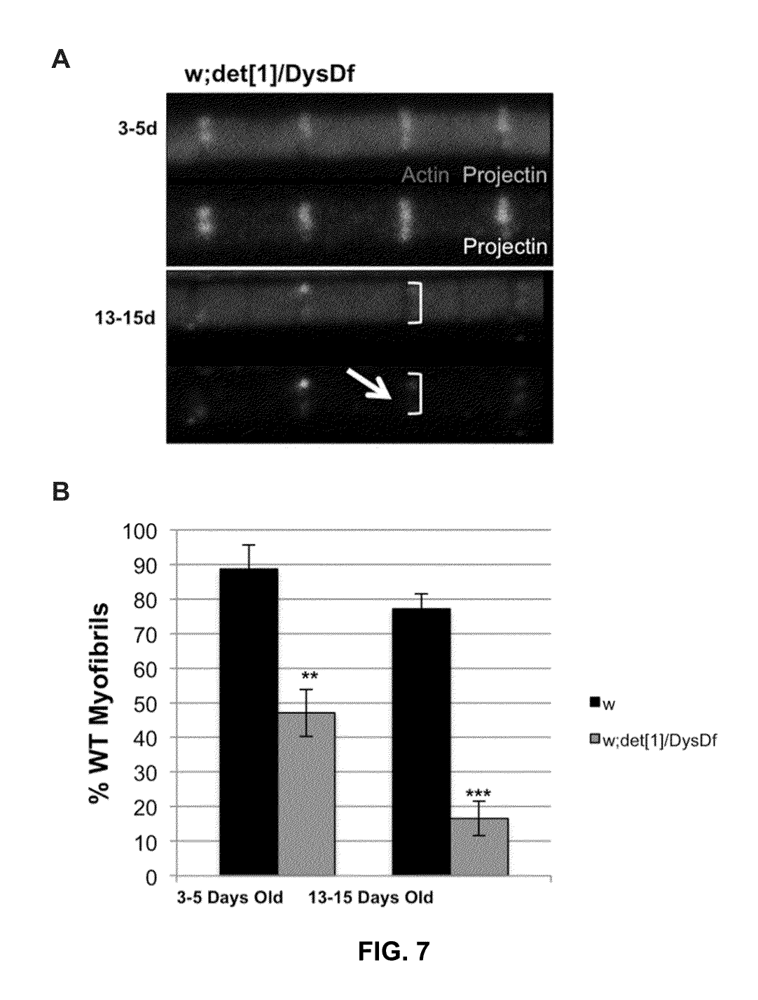

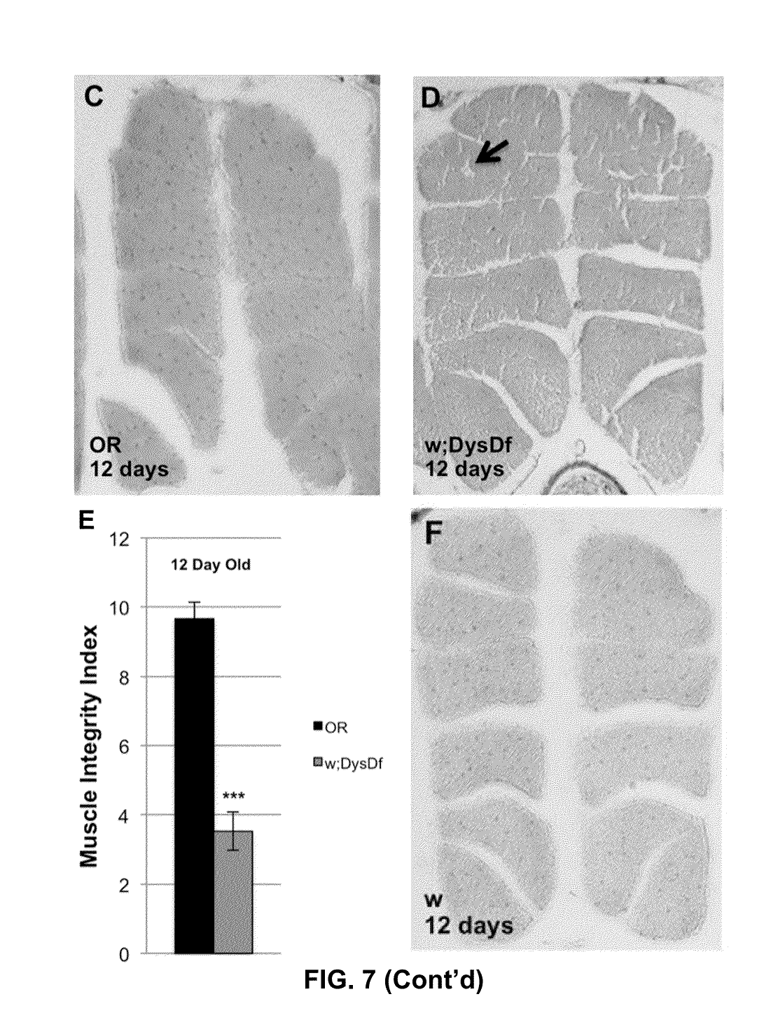

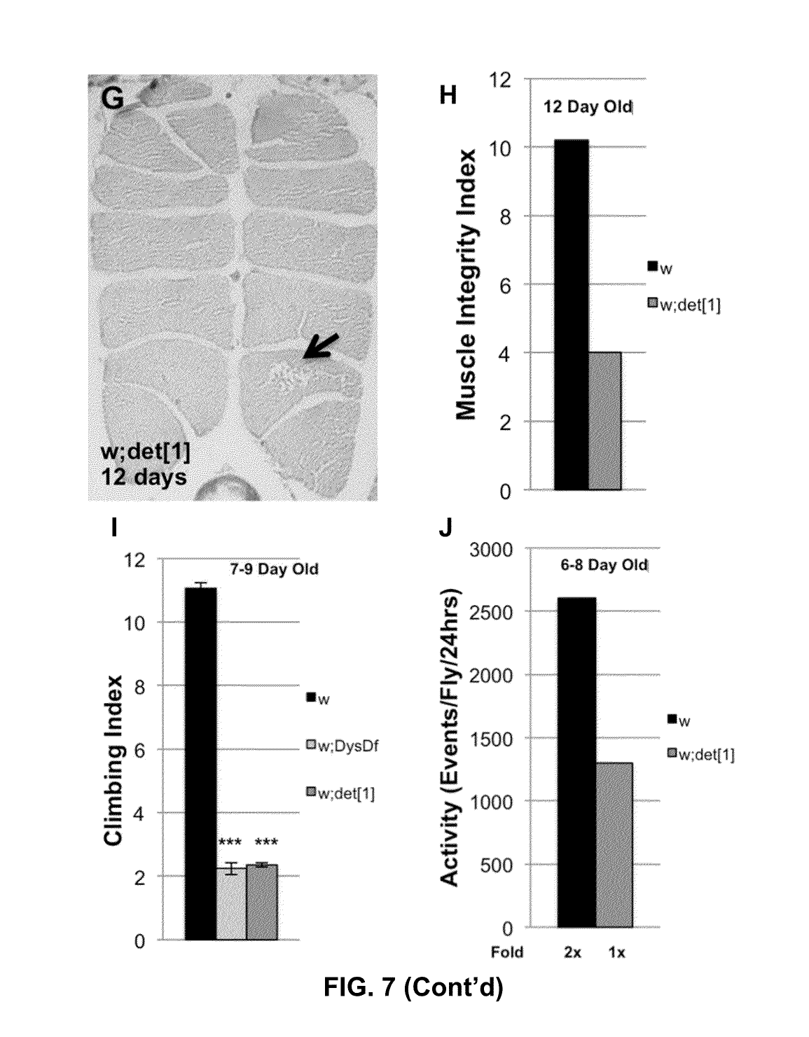

The staining patterns of Projectin were used to assess myofibril integrity in DysDf, a genetic deficiency that removes most of the dystrophin (Dys) gene and Dys^{det1}, a classical mutant allele, along with control wild type flies at two different time points. The Projectin staining in normal myofibrils resulted in a uniform and intense band that spans the entire fibril (FIG. 1a; w 3-5 d). In 3-5 day old Dys mutants at least 40% of the myofibrils had the Projectin pattern already disrupted with DysDf flies showing 80% disruption (FIG. 1b); the phenotypes ranged from a diffuse staining on either side of the Z band to a band that only partly stains the fibril (FIG. 1a, DysDf, Dys^{det1}; FIG. 7a, Dys^{det1}/DysDf). Myofibrils harvested from 13-15 day old Dys mutant flies showed a highly penetrant mutant phenotype in which Projectin staining was largely reduced, punctate, or absent consistent with contraction induced damage over time which is expected in dystrophic flies (FIG. 1a, FIG. 7a).

This defect in the pattern of a structural protein of the muscle contraction machinery in Dys mutants shows that Dystrophin is an essential component in maintaining the stability of the molecular architecture of myofibrils. Significant degradation of titin has also been shown in muscle biopsies of DMD subjects. Collectively, these defective myofibrils eventually degrade and lead to fragmentation and holes that were observed in transverse histological sections (FIG. 7c-h).

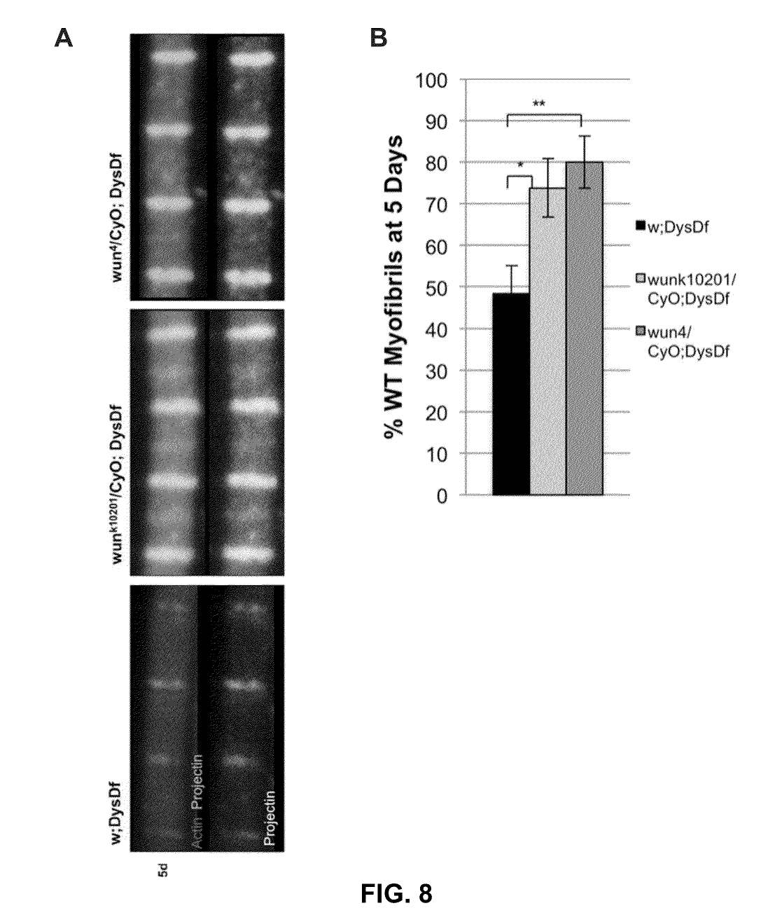

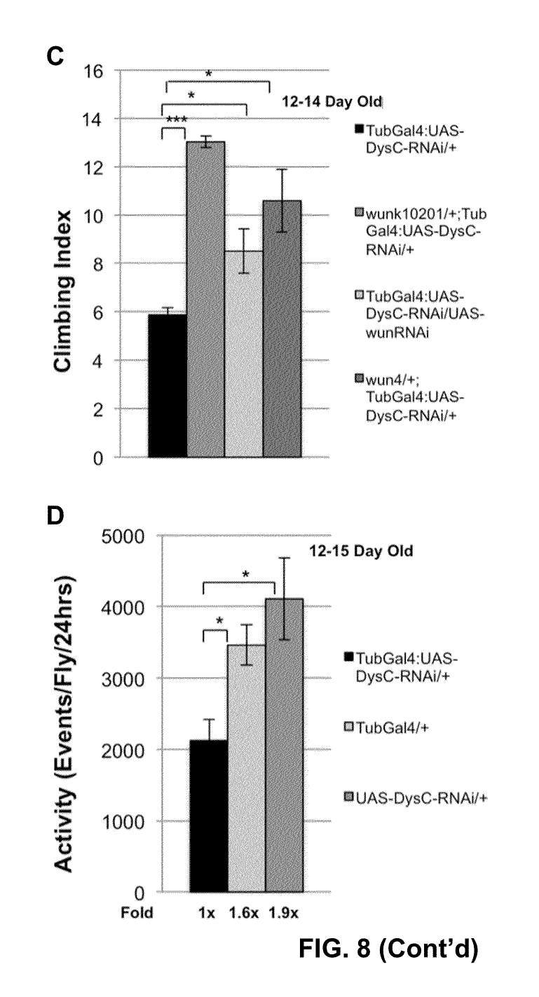

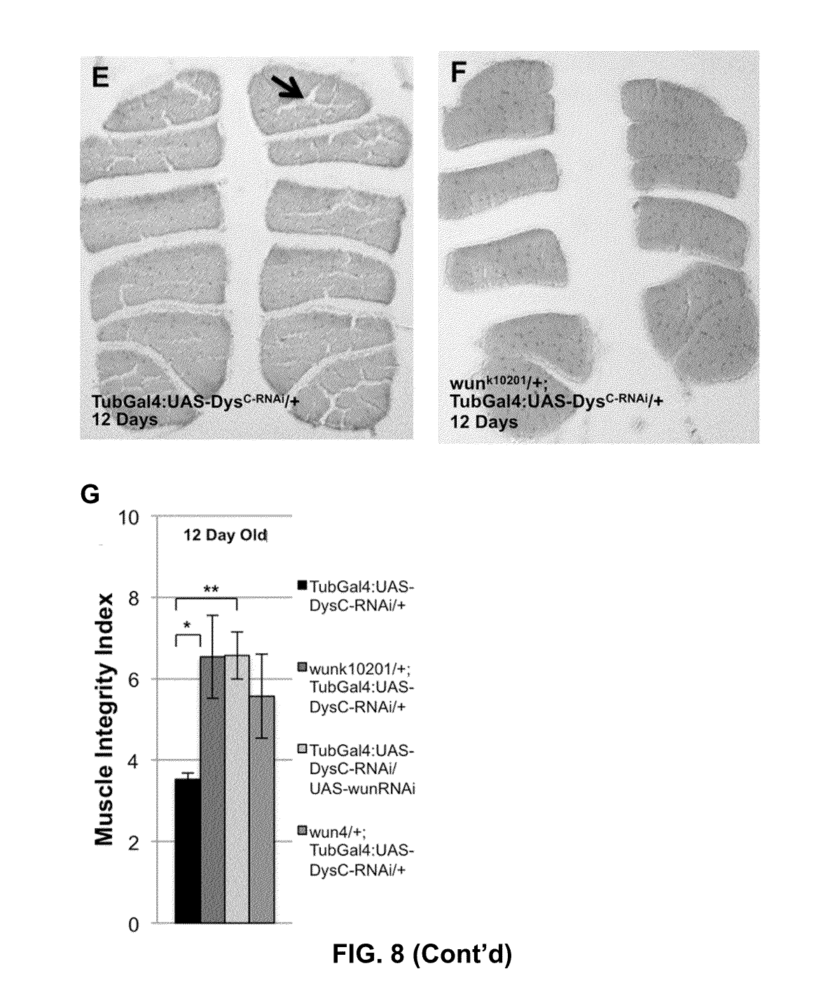

Functionally, dystrophic flies have a reduced climbing ability (FIG. 7i); furthermore the overall activity of dystrophic flies measured continuously over many days is substantially reduced when compared to wild type flies. Activity of dystrophic flies was generally 2-fold less than wild type control flies when tested over different durations of time (FIG. 1e, FIG. 7j, and FIG. 8d).

The protein wunen is a suppressor of the dystrophic wing vein phenotype. The reduction of wunen was tested herein to investigate whether reduced wunen could suppress the defects in myofibril integrity observed in Dys mutants. Since Dystrophin is autosomic in flies and any genetic manipulation requires making the flies homozygous, single chromosome Dys RNAi mutants were used to assess dystrophic suppression. Dys RNAi mutants show an equally penetrant, though milder, defect in Projectin myofibril patterns compared to DysDf mutants (FIG. 1d; TubGal4:UAS-Dys^{C-RNAi}/+).

A significant suppression of the defective, punctate Projectin staining in tubulin-Gal4 driven Dys RNAi myofibrils was observed when wunen was reduced using the wun^{RNAi} allele (FIG. 1d; UASwun^{RNAi}/TubGal4:UAS-Dys^{C-RNAi}). A 4-fold increase was observed in the number of wild type myofibrils in flies, reaching 80% of control levels (FIG. 1e). To determine if this suppression was allele specific, wun^{k10201}, a P element insertion allele and wun⁴, an EMS allele, were all tested. Both alleles showed a suppression of the loss of Projectin in myofibrils from 5 day old wun/CyO; DysDf flies compared to DysDf flies alone (FIG. 8a,b). This correlated with the suppression of the gross morphological defects seen in transverse histological sections (FIG. 8e-g).

The overall activity of the Dys flies with reduced wunen was assessed using automated monitoring. Analysis using tubulin- and actin-Gal4 drivers with multiple wunen alleles (wunk¹⁰²⁰¹, wun^{RNAi} and wun⁴) revealed that over the course of three days, 15 day old dystrophic flies with reduced wunen were significantly more active than Dys mutant flies (FIG. 1f), indicating that reduction in wunen expression suppresses muscle functional defects in dystrophic flies to nearly wild type levels (˜2-fold higher) (FIG. 8d). This correlated with the initial data that reduced wunen increased the climbing ability of dystrophic flies (FIG. 8c).

### Example 2

### Suppression of Dystrophic Muscle and Activity Phenotypes by Increasing S1P Levels

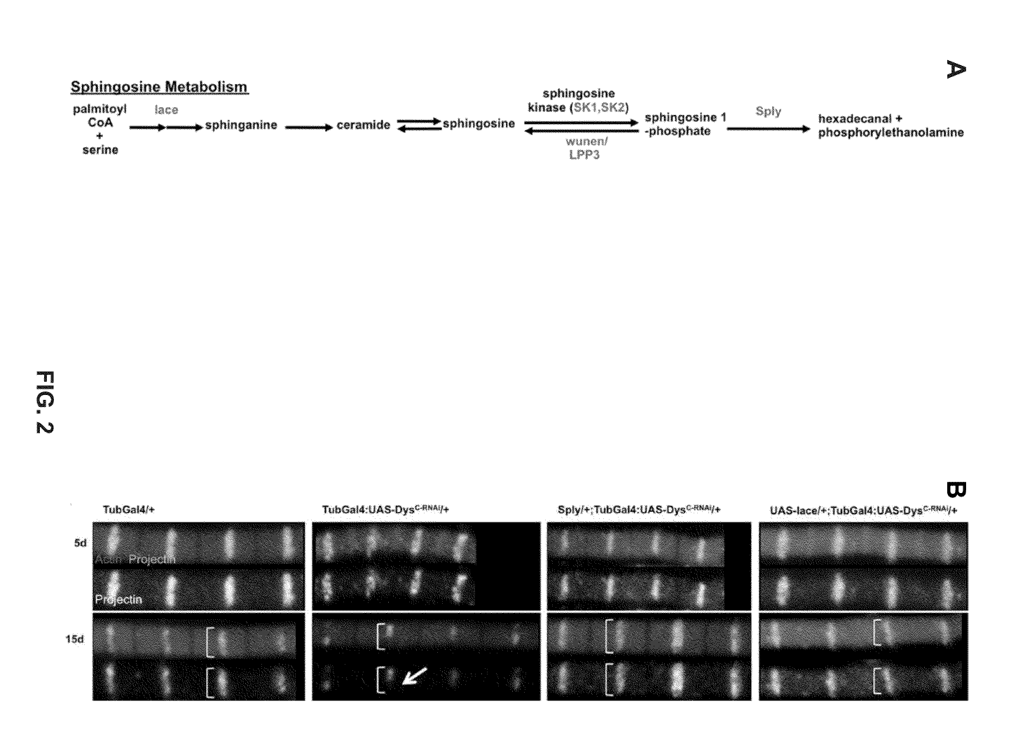

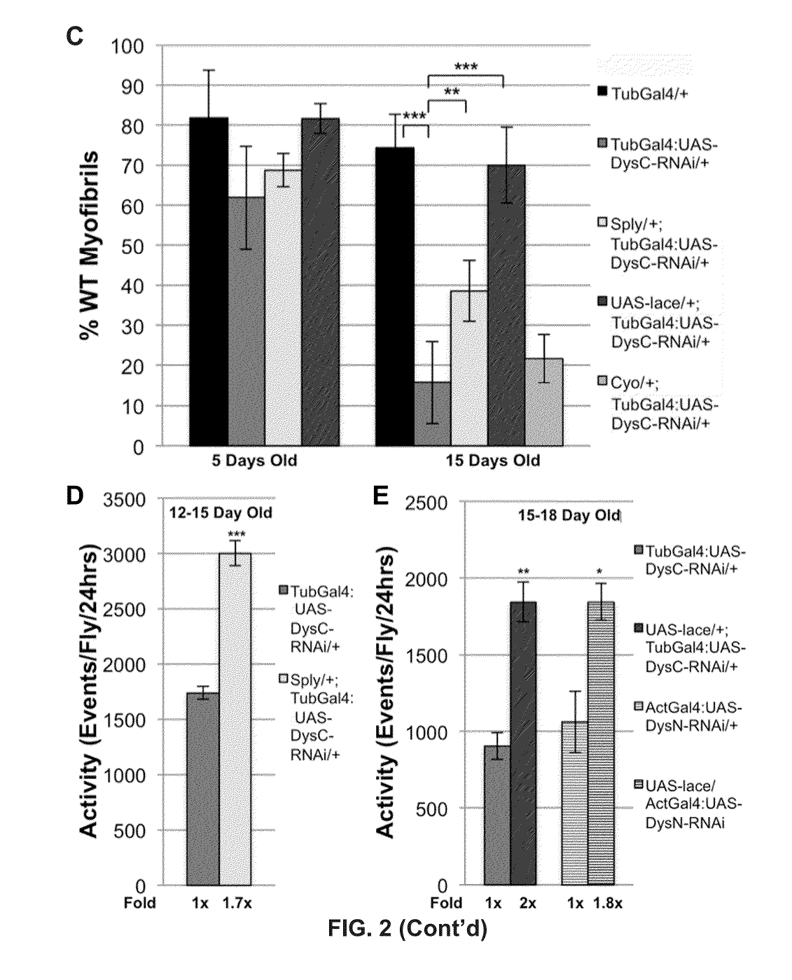

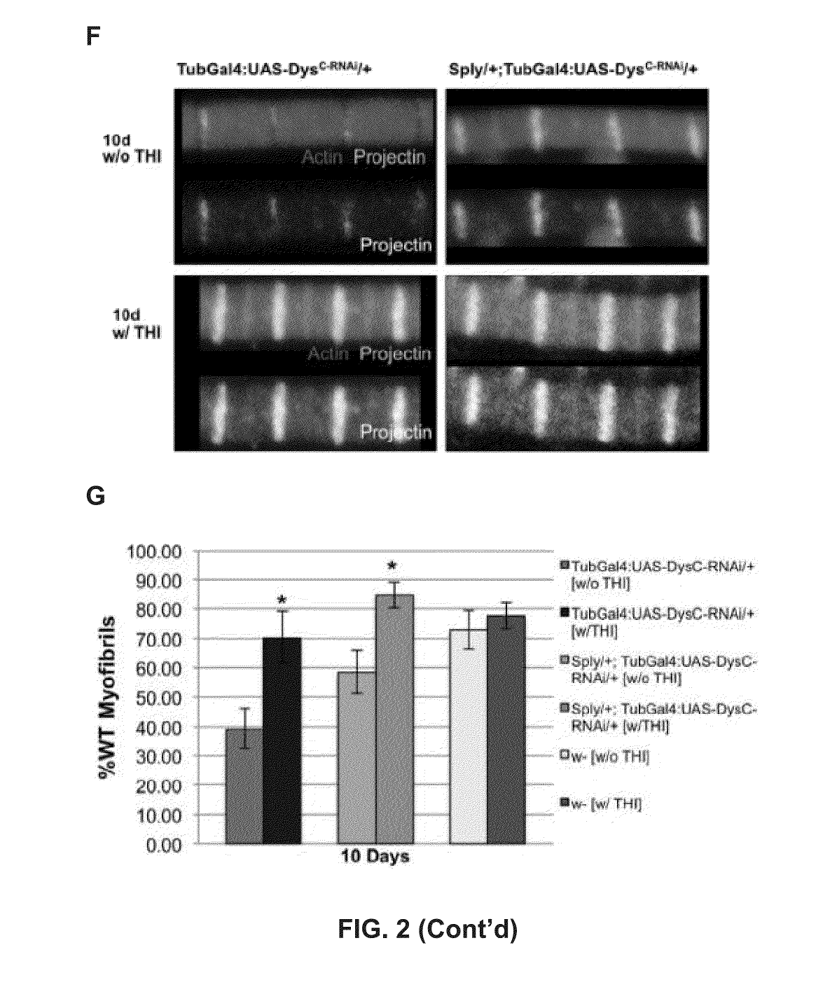

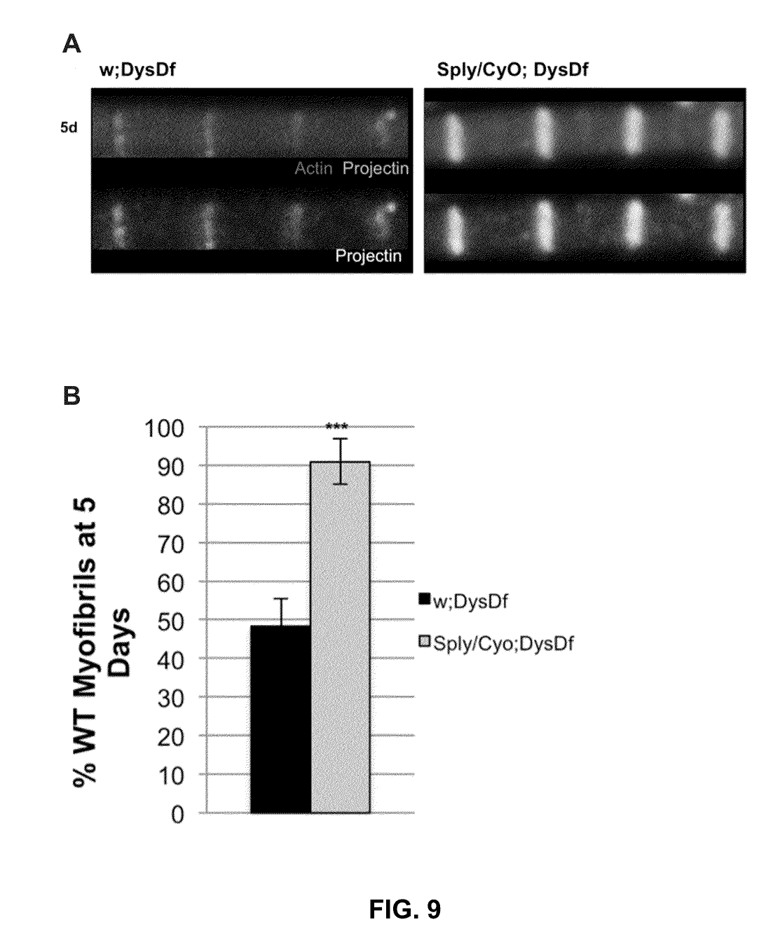

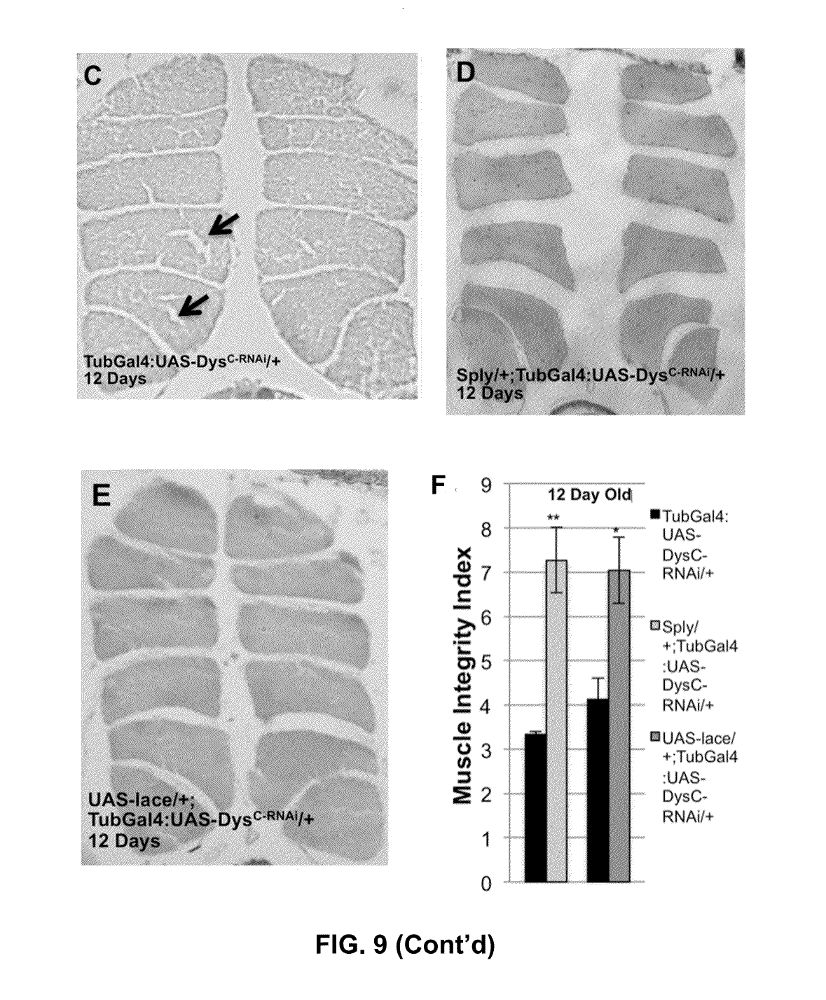

To determine whether wunen dependent suppression of dystrophic phenotypes is a result of increasing S1P, the components of sphingolipid metabolism in dystrophic flies were investigated. Two mutants that, like wunen, would increase S1P levels, were assessed for their ability to suppress dystrophic muscles. The Drosophila S1P lyase gene, Sply, was tested which irreversibly removes S1P from sphingosine metabolism by cleaving the compound to hexadecanal and phosphorylethanolamine (FIG. 2a). In mice, the reduction of this gene leads to an increase in S1P levels. The Sply mutation was placed in flies carrying the tubulin-Gal4 driven Dys RNAi mutant (Sply/+; TubGal4:UAS-Dys^{C-RNAi}) and found a significant suppression of the dystrophic myofibril phenotype as myofibrils isolated from 15 day old dystrophic flies showed a Projectin pattern defect that was significantly rescued by reducing Sply (FIG. 2b,c; Sply/+; TubGal4:UAS-Dys^{C-RNAi}). Suppression of the dystrophic phenotype was observed in myofibrils from 5 day old DysDf flies with reduced Sply (FIG. 9a,b; Sply/CyO; DysDf/DysDf). This correlated with a reduction of muscle fragmentation observed in transverse histological sections (FIG. 9c,d,f). Furthermore, automated monitoring revealed a significant increase in activity when Sply was reduced in dystrophic flies (FIG. 2d).

Using the UAS-lace over-expression construct, S1P was increased by upregulating the expression of the first gene in the de novo synthesis pathway, lace, the Drosophila serine palmitoyl CoA transferase (FIG. 2a). Over-expressed UAS-lace in the tubulin-Gal4 driven Dys RNAi background revealed that myofibrils observed at 15 days showed a significant suppression, nearly to wild type, of the dystrophic phenotype (FIG. 2b,c; UAS-lace/+; TubGal4:UAS-Dys^{C-RNAi}/+). Similar to reduced Sply, the fragmentation defect observed in histological sections of dystrophic flies was significantly suppressed (FIG. 9c,e,f). Finally, dystrophic flies with UAS-lace either tubulin-Gal4 or actin-Gal4 driven were significantly more active than the dystrophic flies without lace over-expression (FIG. 2e).

In mice, the genetic reduction of S1P lyase activity can be phenocopied pharmacologically by the delivery of the small molecule 1-[5-[(1R,2S,3R)-1,2,3,4-tetrahydroxybutyl]-1H-imidazol-2-yl]-ethanone (THI), which inhibits the activity of S1P lyase. Dystrophic mutant flies were administered THI to investigate possible THI suppression of the muscle degeneration phenotype. After 3 days of a once-daily oral regimen of THI, myofibrils from 10 day old flies were harvested and analyzed, revealing a significant suppression of muscle wasting (FIG. 2f,g). These results show that an increase in S1P levels, induced either genetically or pharmacologically, result in a significant rescue of the dystrophic muscle phenotype observed in flies.

### Example 3

### Improved Muscle Regeneration, Increased Muscle Fiber Size, Increased Satellite Cell Numbers, and Decreased Muscle Fat Deposits Following Acute Injury in Mdx Mice after Administration of the S1P Lyase Inhibitor THI

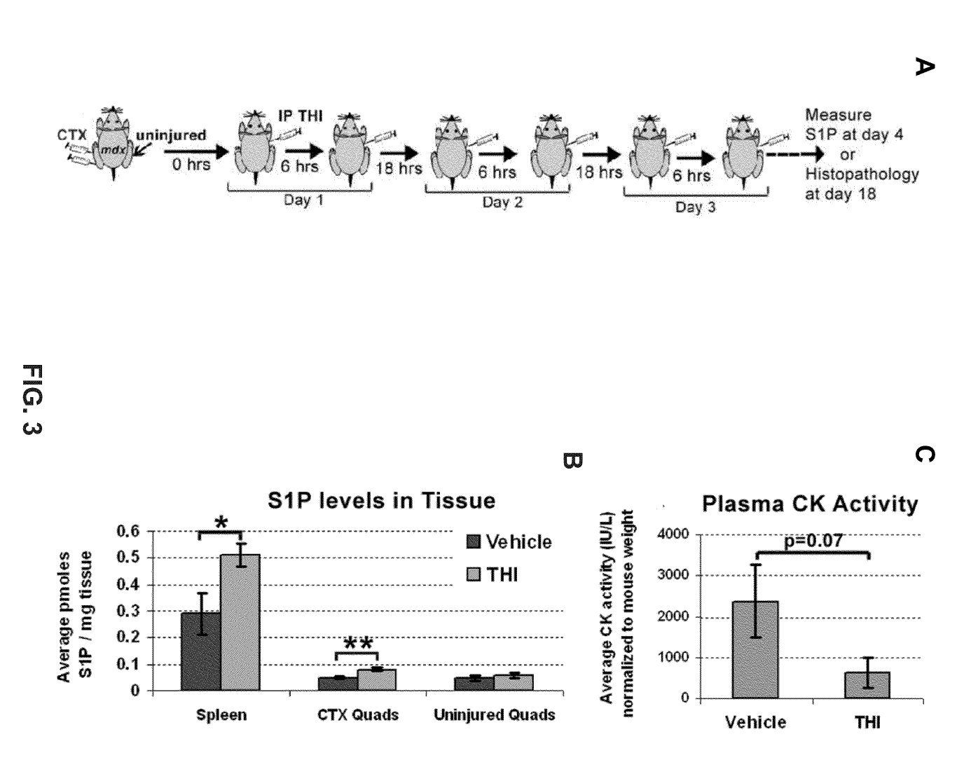

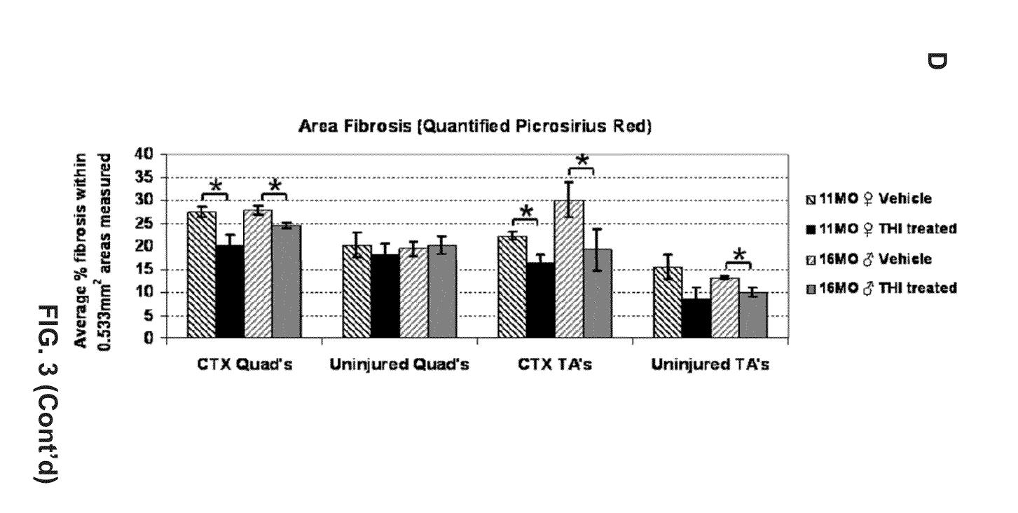

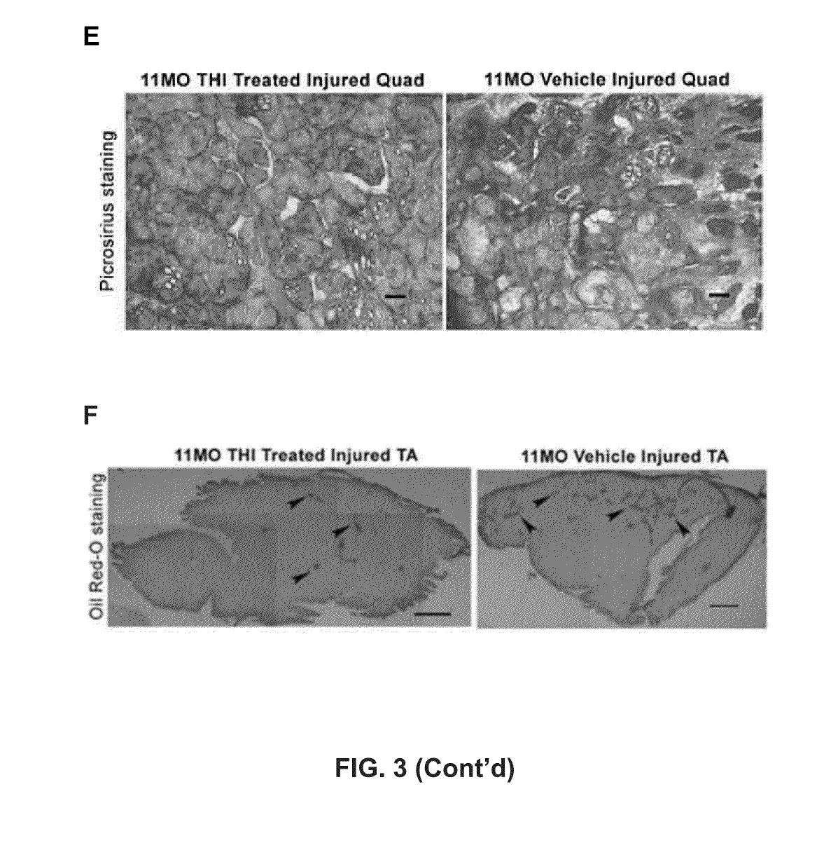

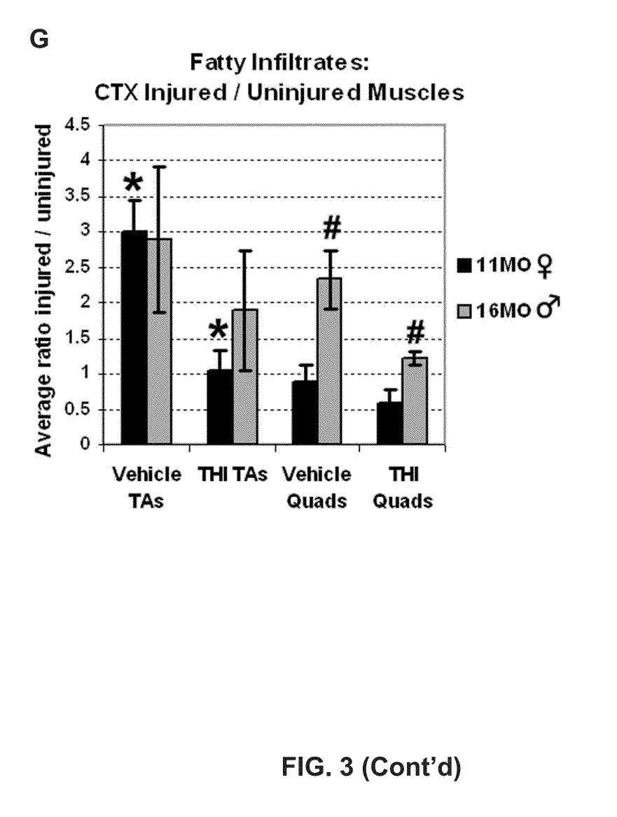

The effects of THI in the dystrophic mdx mouse model were studied. The mdx mice include the X chromosome-linked mdx mutation which produces animals that lack a functional dystrophin protein and possess histological muscle lesions similar to human muscular dystrophy. THI was administered intraperitoneally (IP) to three groups of mdx mice. Right tibialis anterior (TA) and quadriceps muscles were uninjured, while left counterparts were injured using CTX, a well characterized model of acute injury where initial muscle destruction is followed by a rapid myogenic response. The mdx mice were injected with THI, or PBS as a vehicle control, immediately following injury with CTX 5 times during a 3-day period (FIG. 3a). In the first group (N=6, 5-month-old males) the mdx mice were sacrificed at day 4 to analyze the efficacy of the administration of THI for increasing S1P levels in spleen, CTX injured quadriceps, and uninjured quadriceps. The effects of THI administration on the plasma creatine kinase (CK) activity level was also measured. As shown in FIG. 3b, the administration of THI significantly increased the S1P level in spleen tissue and CTX injured quadriceps (*p<0.05, **p<0.01). FIG. 3c shows that THI administration reduced plasma CK levels in THI treated mice compared to vehicle treated mice. Because high plasma CK levels are a clinical hallmark of DMD pathology in human and mice, the data suggest that THI administration has a beneficial effect in mdx mice muscle pathology.

Muscle repair in mdx mice become impaired during aging resulting in muscle atrophy and dystrophy. The effects of THI on the histopathology were assessed in injured and uninjured muscles from two groups of aged mdx mice (n=6, 11 month-old females and n=7, 16 month-old males). In this experiment, right TAs and quadriceps muscles were uninjured, while left counterparts were injured using CTX. The mdx mice were injected with THI, or PBS as a vehicle control, immediately following injury with CTX 5 times during a 3-day period. Injured and uninjured muscles were harvested 18 days post injury to survey the effects of THI on muscle repair. FIG. 16 shows the ratio of dry weight from each CTX injured muscle to dry weight of the uninjured muscles, normalized to the body weight of the 16 month-old (MO) mdx male mice. Injured muscles were lighter than uninjured muscles in vehicle treated mice, an approximate weight loss of 20% (*p<0.05). In the THI treated mice, the weight of injured quadriceps was similar to uninjured quadriceps (ratio close to one) indicating that THI administration protects from muscle wasting during injury.

The levels of fibrosis and fat deposition were quantified, both hallmarks of dystrophic muscle pathology, and it was found that treatment with THI significantly reduced fibrosis in injured TAs and quadriceps muscles (FIG. 3d). Along with a reduction in fibrosis, the overall histological muscle morphology appears more organized in the injured muscles of THI treated animals (FIG. 3e).

To test whether THI treated mice show decreased fat deposition in muscles compared to controls, the fat deposits were quantified using cross sections of THI and vehicle treated muscles (FIG. 3f). The ratio of fat deposits in injured over uninjured contralateral muscles was then compared for THI and vehicle treated mice (FIG. 3g). The average fat deposition in injured TAs was 3-fold higher than uninjured contralateral TAs (FIG. 3g). Whereas the THI-treated injured TAs had a significantly lower average fat deposition (FIG. 3g).

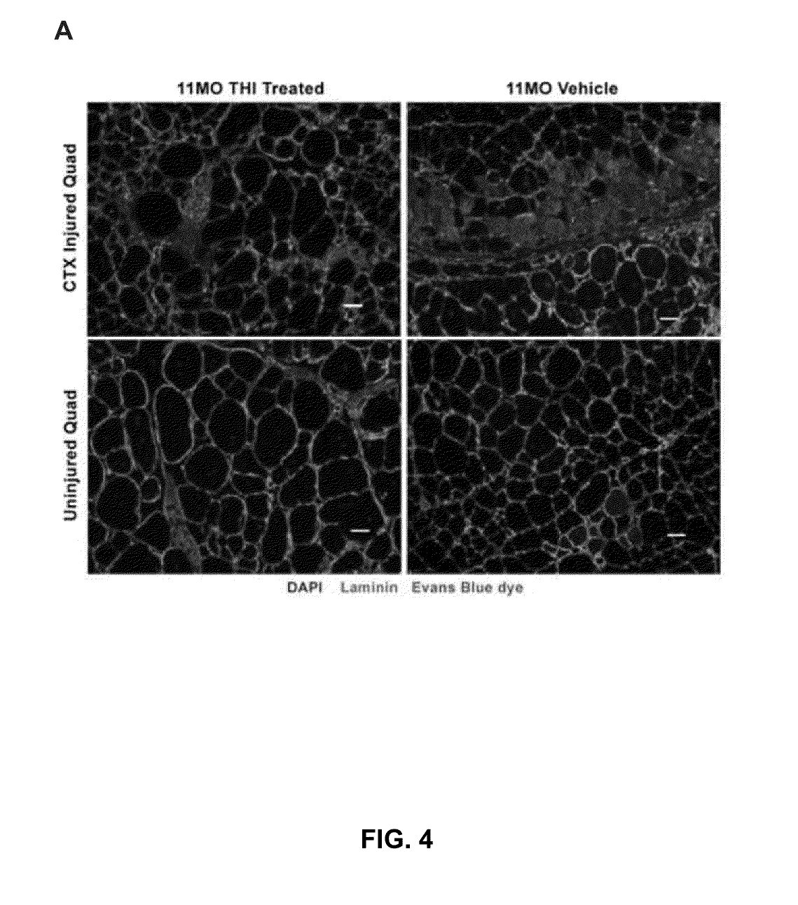

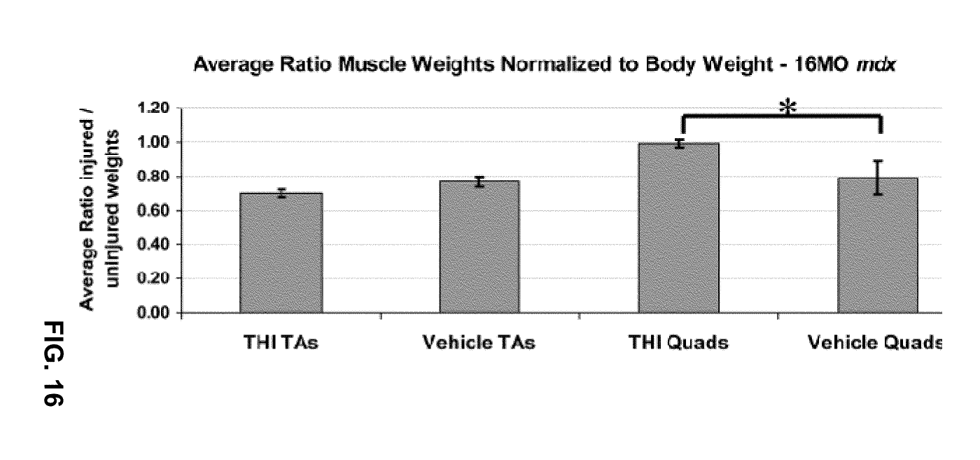

Further analysis of THI treated mice revealed an increase in quadriceps and diaphragm muscle fiber size (FIG. 4a). Although mdx mice are associated with muscle hypertrophy compared to wild type, a significant increase was observed in the minimum fiber diameter with THI treatment in both uninjured and injured quadriceps and the diaphragms of 11 month-old mice (FIG. 4b, c and FIG. 17). Uninjured quadriceps of THI-treated 16 month-old males also showed a significant increase in muscle fiber size (FIG. 4D).

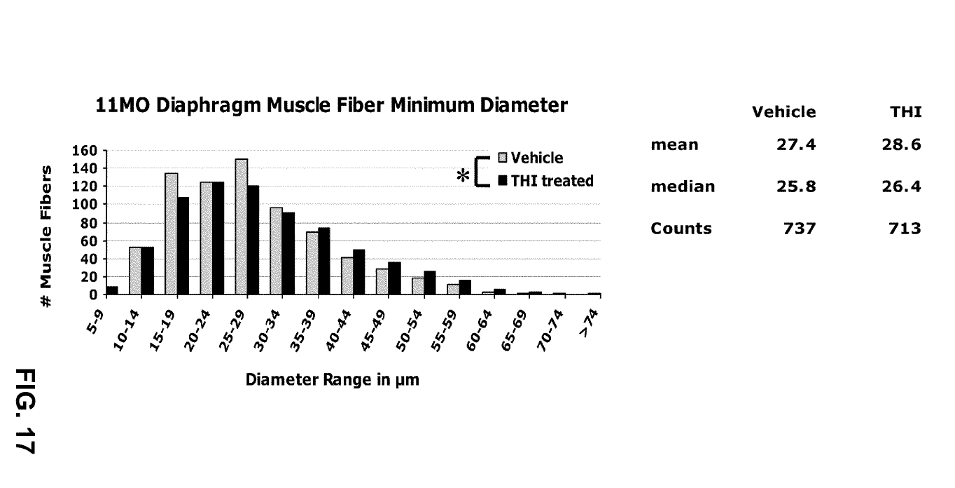

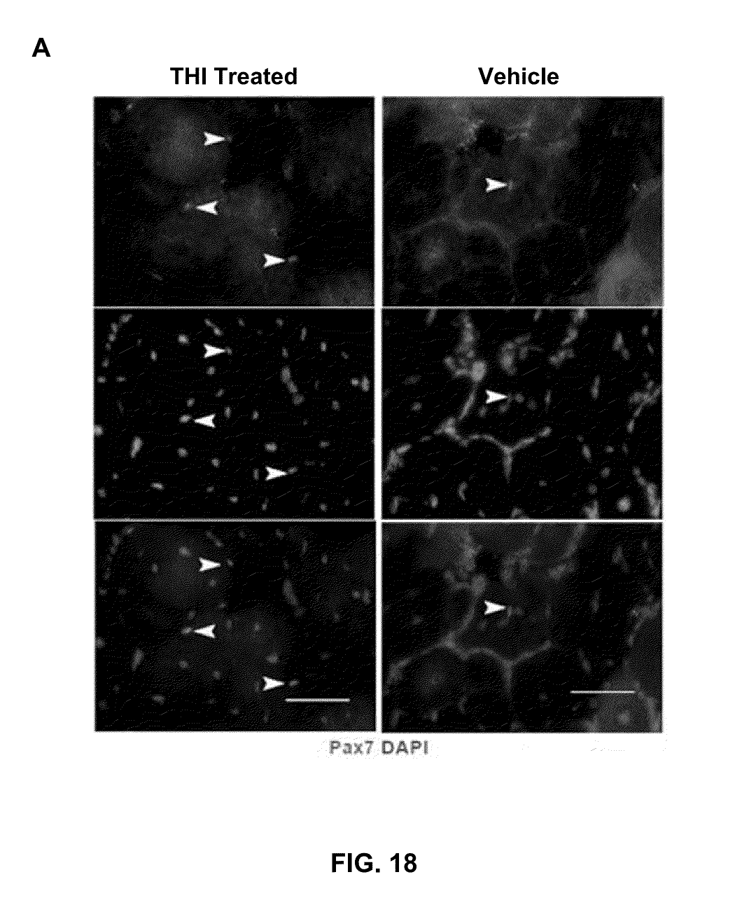

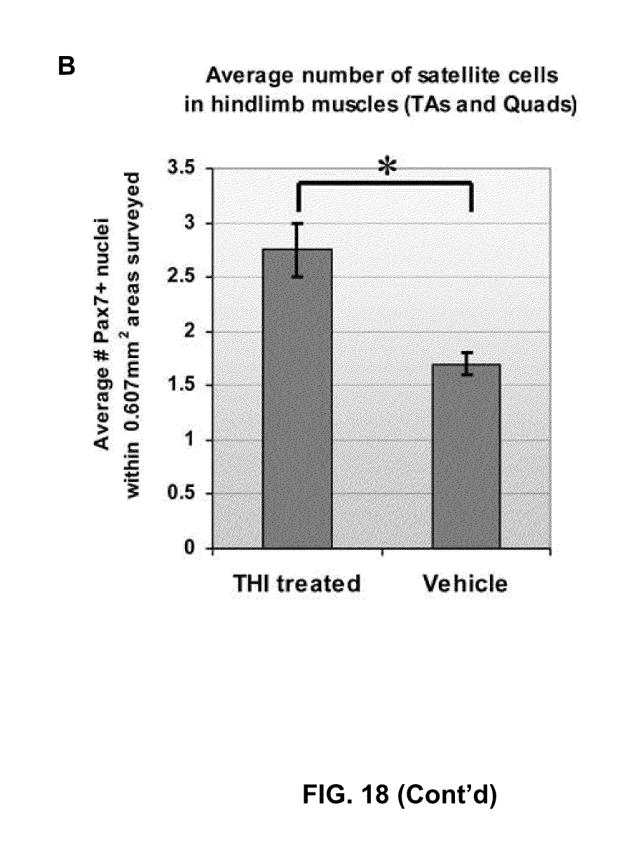

To assess if increases in muscle fiber size observed with THI treatment are accompanied by an increase in the number of satellite cells, the number of Pax7 positive cells was quantified. Pax7 is a specific marker of satellite cells, which decline in older mdx miscles. As shown in FIG. 18a, few satellite cells (Pax7+nuclei) were visible in cross-sections of 11 month-old mdx muscles. However, there was a significant increase in the mean number of satellite cells (Pax7+nuclei) in limb muscles (TAs and Quadriceps) of THI treated 11 month-old mice (FIGS. 18a and b).

The reduction in fibrosis and fat deposition, and increase in myofiber size and satellite cell number, indicate that increasing systemic levels of S1P via THI treatment has a profound benefit in dystrophic muscle regeneration.

### Example 4

### Administration of Derivatives of THI Improve Muscle Regeneration, Increase Muscle Fiber Size, and Decrease Muscle Fat Deposits in the mdx Mouse

The effects of derivatives of THI in dystrophic mice are studied. The following derivatives of THI are administered: (1R,2S,3R)-1-(2-(5-methylisoxazol-3-yl)-1H-imidazol-5-yl)butane-1,2,3,4-tetraol; (1R,2S,3R)-1-(2-(5-ethylisoxazol-3-yl)-1H-imidazol-5-yl)butane-1,2,3,4-tetraol; (1R,2S,3R)-1-(2-(isoxazol-3-yl)-1H-imidazol-5-yl)butane-1,2,3,4-tetraol; (1R,2S,3R)-1-(2-(isoxazol-3-yl)-1H-imidazol-4-yl)butane-1,2,3,4-tetraol; (1R,2S,3R)-1-(2-(2-methylthiazol-4-yl)-1H-imidazol-4-yl)butane-1,2,3,4-tetraol; (1R,2S,3R)-1-(2-(1-benzyl-1H-1,2,4-triazol-3-yl)-1H-imidazol-4-yl)butane-1,2,3,4-tetraol hydrochloride; (1R,2S,3R)-1-(1H,1′H-2,2′-biimidazol-5-yl)butane-1,2,3,4-tetraol; (1R,2S,3R)-1-(2-(5-methoxy-4,5-dihydroisoxazol-3-yl)-1H-imidazol-5-yl)butane-1,2,3,4-tetraol; (1R,2S,3R)-1-(2-(5-methyl-1H-pyrazol-3-yl)-1H-imidazol-5-yl)butane-1,2,3,4-tetraol; 1-(4-((1R,2S,3R)-1,2,3,4-tetrahydroxybutyl)-1H-imidazol-2-yl)-ethanone oxime; (E)-1-(4-((1R,2S,3R)-1,2,3,4-tetrahydroxybutyl)-1H-imidazol-2-yl)-ethanone oxime; (Z)-1-(4-((1R,2S,3R)-1,2,3,4-tetrahydroxybutyl)-1H-imidazol-2-yl)-ethanone oxime; 1-(4-((1R,2S,3R)-1,2,3,4-tetrahydroxybutyl)-1H-imidazol-2-yl)ethanone O-methyl oxime; (E)-1-(4-((1R,2S,3R)-1,2,3,4-tetrahydroxybutyl)-1H-imidazol-2-yl)ethanone O-methyl oxime; (Z)-1-(4-((1R,2S,3R)-1,2,3,4-tetrahydroxybutyl)-1H-imidazol-2-yl)ethanone O-methyl oxime; N′-(1-(4-((1R,2S,3R)-1,2,3,4-tetrahydroxybutyl)-1H-imidazol-2-yl)ethylidene)acetohydrazide; 4-methyl-N′-(1-(4-((1R,2S,3R)-1,2,3,4-tetrahydroxybutyl)-1H-imidazol-2-yl)ethylidene)benzenesulfonohydrazide; (E)-4-methyl-N′-(1-(4-((1R,2S,3R)-1,2,3,4-tetrahydroxybutyl)-1H-imidazol-2-yl)ethylidene)benzenesulfonohydrazide; (Z)-4-methyl-N′-(1-(4-((1R,2S,3R)-1,2,3,4-tetrahydroxybutyl)-1H-imidazol-2-yl)ethylidene)benzenesulfonohydrazide; N′-(1-(4-((1R,2S,3R)-1,2,3,4-tetrahydroxybutyl)-1H-imidazol-2-yl)ethylidene)benzohydrazide; ethyl 2-(1-(4-((1R,2S,3R)-1,2,3,4-tetrahydroxybutyl)-1H-imidazol-2-yl)ethylidene)hydrazinecarboxylate; (E)-ethyl 2-(1-(4-((1R,2S,3R)-1,2,3,4-tetrahydroxybutyl)-1H-imidazol-2-yl)ethylidene)hydrazinecarboxylate; (Z)-ethyl 2-(1-(4-((1R,2S,3R)-1,2,3,4-tetrahydroxybutyl)-1H-imidazol-2-yl)ethylidene)hydrazinecarboxylate; N′-(1-(4-((1R,2S,3R)-1,2,3,4-tetrahydroxybutyl)-1H-imidazol-2-yl)ethylidene)nicotinohydrazide; (E)-N′-(1-(4-((1R,2S,3R)-1,2,3,4-tetrahydroxybutyl)-1H-imidazol-2-yl)ethylidene)nicotinohydrazide; (Z)—N′-(1-(4-((1R,2S,3R)-1,2,3,4-tetrahydroxybutyl)-1H-imidazol-2-yl)ethylidene)nicotinohydrazide; 3-chloro-N′-(1-(4-((1R,2S,3R)-1,2,3,4-tetrahydroxybutyl)-1H-imidazol-2-yl)ethylidene)benzohydrazide; 4-fluoro-N′-(1-(4-((1R,2S,3R)-1,2,3,4-tetrahydroxybutyl)-1H-imidazol-2-yl)ethylidene)benzohydrazide; (E)-4-fluoro-N′-(1-(4-((1R,2S,3R)-1,2,3,4-tetrahydroxybutyl)-1H-imidazol-2-yl)ethylidene)benzohydrazide; (Z)-4-fluoro-N′-(1-(4-((1R,2S,3R)-1,2,3,4-tetrahydroxybutyl)-1H-imidazol-2-yl)ethylidene)benzohydrazide; 6-amino-N′-(1-(4-((1R,2S,3R)-1,2,3,4-tetrahydroxybutyl)-1H-imidazol-2-yl)ethylidene)nicotinohydrazide; (E)-6-amino-N′(1-(4-((1R,2S,3R)-1,2,3,4-tetrahydroxybutyl)-1H-imidazol-2-yl)ethylidene)nicotinohydrazide; (Z)-6-amino-N′-(1-(4-((1R,2S,3R)-1,2,3,4-tetrahydroxybutyl)-1H-imidazol-2-yl)ethylidene)nicotinohydrazide; N′-(1-(4-((1R,2S,3R)-1,2,3,4-tetrahydroxybutyl)-1H-imidazol-2-yl)ethylidene)isonicotinohydrazide; (E)-N′-(1-(4-((1R,2S,3R)-1,2,3,4-tetrahydroxybutyl)-1H-imidazol-2-yl)ethylidene)isonicotinohydrazide; (Z)—N′-(1-(4-((1R,2S,3R)-1,2,3,4-tetrahydroxybutyl)-1H-imidazol-2-yl)ethylidene)isonicotinohydrazide; N′-(1-(4-((1R,2S,3R)-1,2,3,4-tetrahydroxybutyl)-1H-imidazol-2-yl)ethylidene)biphenyl-3-carbohydrazide; (E)-N′-(1-(4-((1R,2S,3R)-1,2,3,4-tetrahydroxybutyl)-1H-imidazol-2-yl)ethylidene)biphenyl-3-carbohydrazide; and (Z)—N′-(1-(4-((1R,2S,3R)-1,2,3,4-tetrahydroxybutyl)-1H-imidazol-2-yl)ethylidene)biphenyl-3-carbohydrazide.

The derivatives of THI are administered intraperitoneally (IP). The response to muscle injury is assessed in older mdx mice to determine the effects of the derivatives of THI and the resulting increasing levels of S1P in dystrophic animals at a stage of severe muscle atrophy. Left tibialis anterior (TA) and quadriceps muscles are injured by local administration of the cytotoxic agent cardiotoxin (CTX), a well characterized model of acute injury where initial muscle destruction is followed by a rapid myogenic response which is impaired in old mdx mice. Contralateral TA and quadriceps muscles are uninjured. Animals are injected peritoneally immediately following CTX injury, and 8 hours post-injury, with either the derivatives of THI or PBS as a vehicle control. Injured and uninjured muscles are harvested 18 days post-injury to survey the effects of the derivatives of THI in muscle tissue.

The level of fibrosis and fat deposition is also quantified, which are generally elevated in dystrophic muscle pathology. The results show that administration of derivatives of THI significantly reduces muscle fibrosis in injured TA and quadriceps muscles. Administration of derivatives of THI also results a more organized histological muscle morphology compared to the injured muscles in the untreated mice.

To observe the effects of THI derivatives on fat deposition in muscles, the fat deposits in muscle are quantified using cross sections of THI derivative-treated muscles and vehicle-treated muscles. The ratio of fat deposits in injured over uninjured muscles is then compared for THI derivative and vehicle-treated mice. In the control animals, average fat deposition in injured TAs is approximately 3-fold higher than uninjured contralateral TAs. However, injured muscles treated with derivatives of THI show lower muscle fat deposits and an increase in muscle fiber size and number. Furthermore, an increase is observed in the minimum fiber diameter after treatment with derivatives of THI in uninjured quadriceps and the diaphragm. Overall, the results show that increasing systemic levels of S1P via treatment with derivatives of THI causes a reduction in muscle fibrosis and fat deposition, and an increase in muscle fiber size and number.

### Example 5

### Direct Administration of S1P Promotes Muscle Regeneration in mdx Mice Following Cardiotoxin Injury

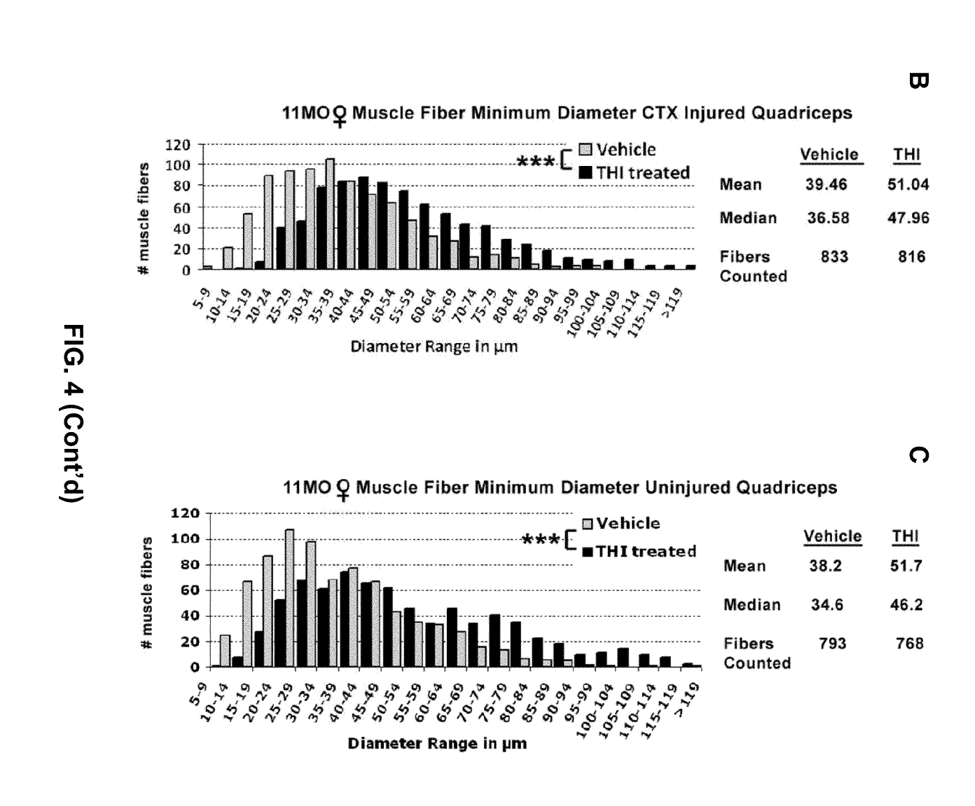

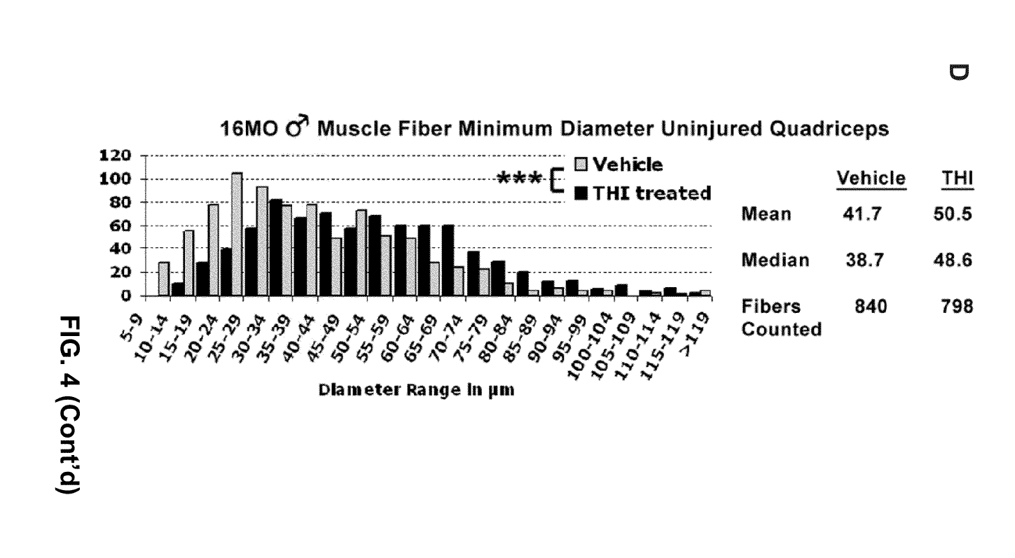

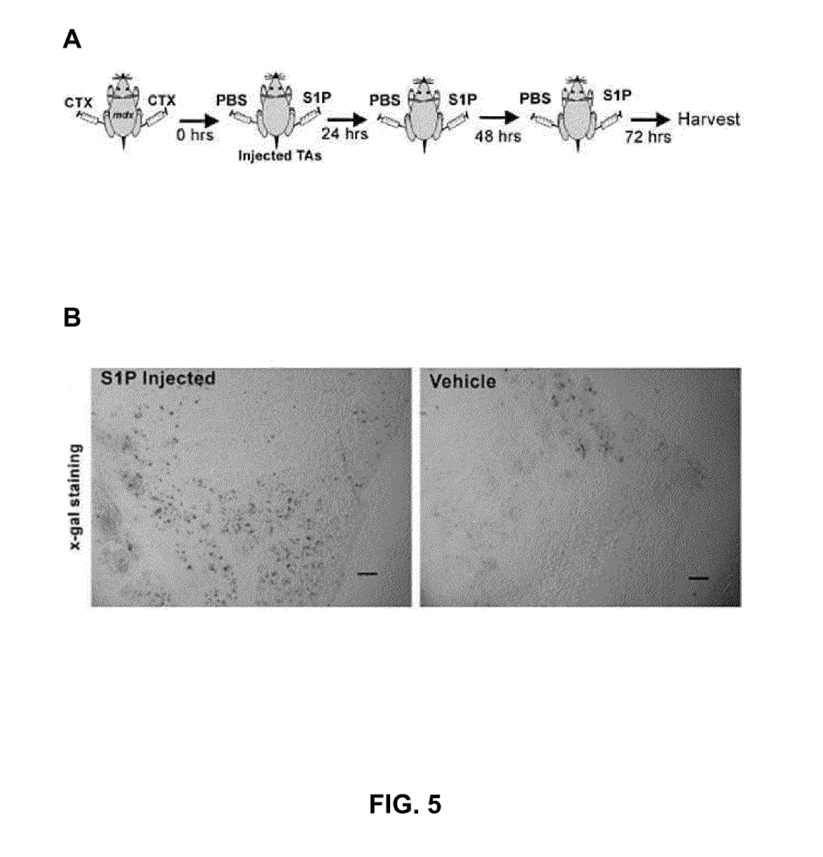

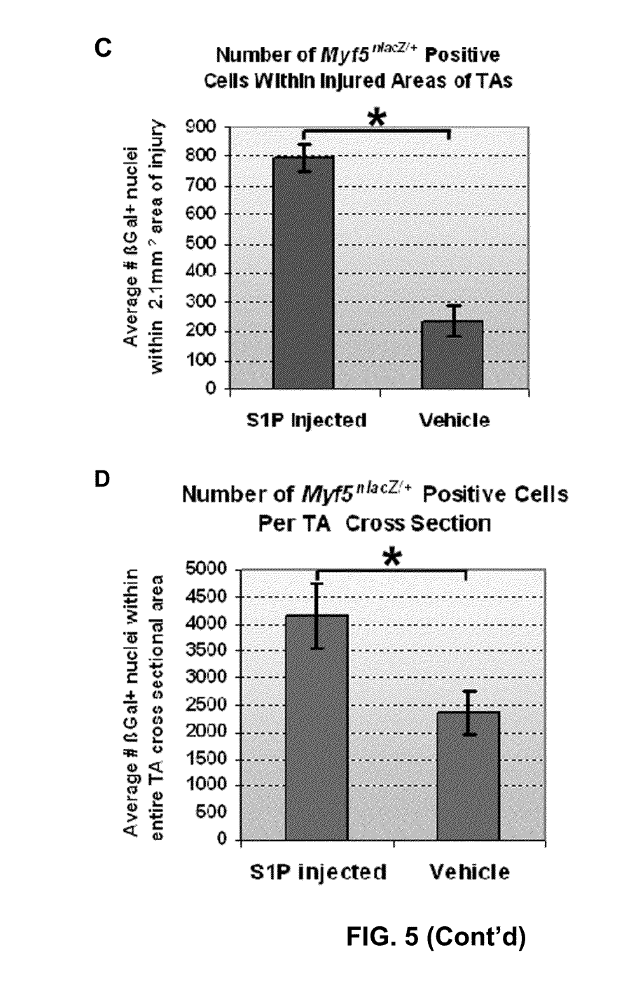

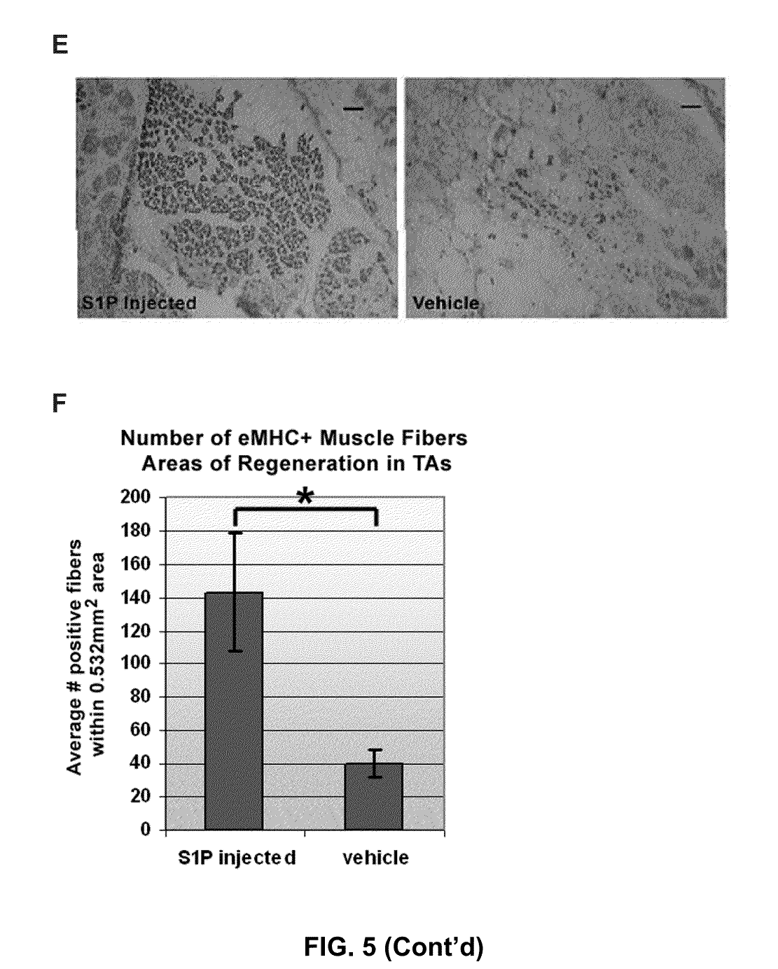

S1P is involved in muscle satellite cell turnover, myoblast differentiation, and muscle regeneration in non-diseased mice. Drosophila do not appear to harbor adult muscle satellite cells, thus the S1P based suppression studies were performed with the mdx mouse, where satellite cell response to altered S1P levels could be measured. The effects of S1P treatment were examined following CTX induced acute injury in dystrophic muscles. To identify myogenic cells, mdx:myf5^{nlacz/+} mice were used carrying the nuclear lacZ reporter driven by the endogenous Myf5 gene. CTX was applied to both TA muscles (n=3 mice, 3 month-old mdx:myf5^{nlacz/+} males), then S1P was injected intramuscularly into left TAs and a vehicle control into right TAs daily for the first three days following injury. TAs were harvested on day 4 post injury for analysis of β-galactosidase positive (Myf5+) nuclei. S1P treated muscles show a dramatic four-fold increase in the numbers of Myf5+ nuclei in areas with severe CTX damage in S1P treated TAs as compared to vehicle controls (FIG. 5b,c; FIG. 10). Furthermore, S1P administration yielded a gross increase of Myf5+ cells for the entire cross-sectional area of each treated TA (FIG. 5d). These data demonstrate that S1P treatment increases the number of myogenic cells in mdx muscles following injury and suggests that S1P promotes satellite cell proliferation in vivo.

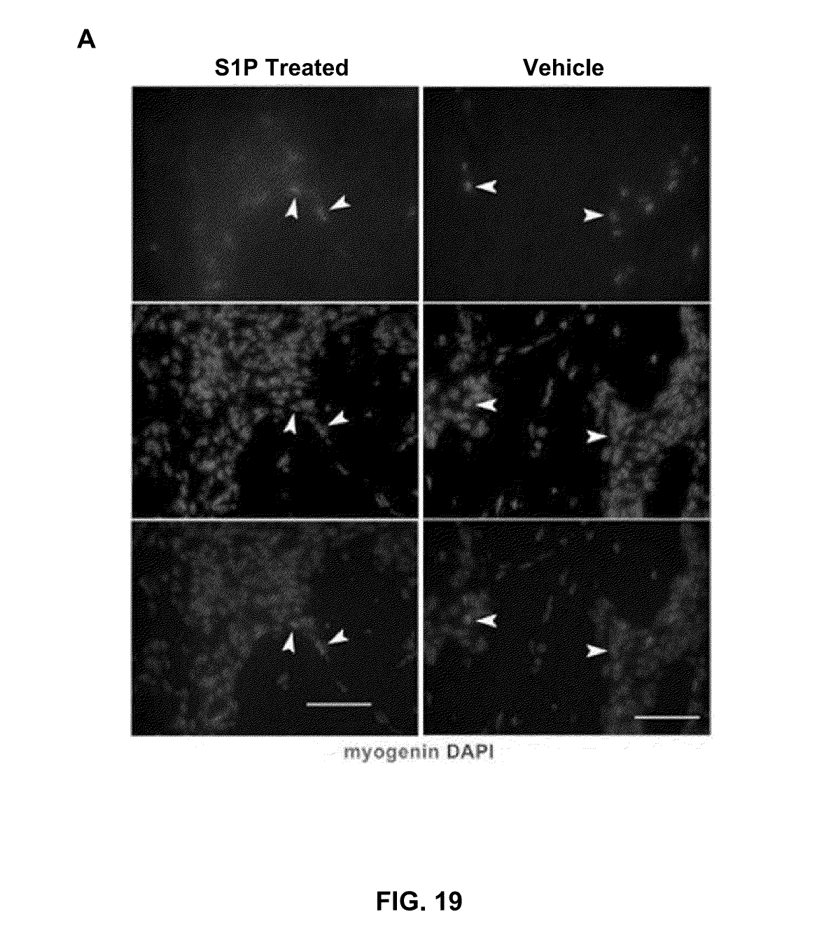

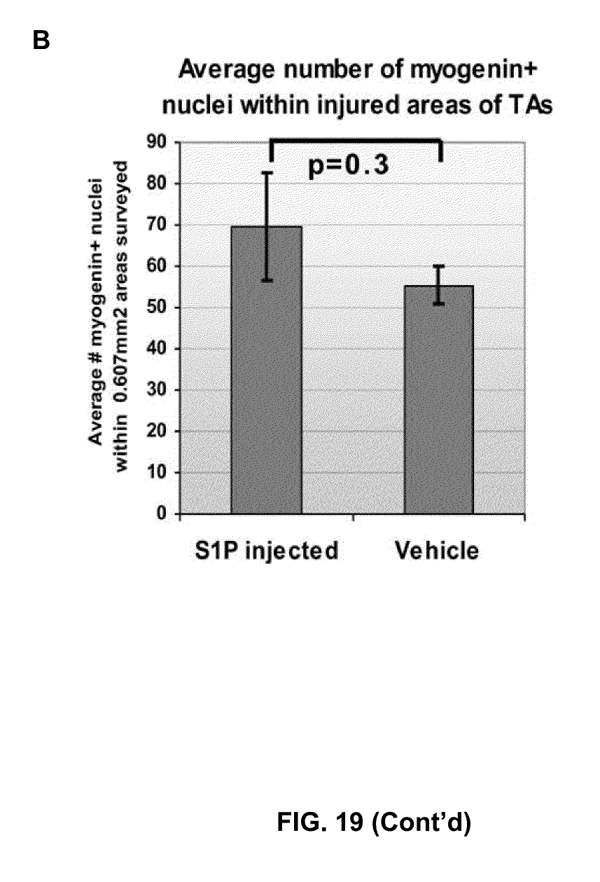

The number of myogenin positive nuclei was quantified to discern if the increase in Myf5+ nuclei represented a rise in activated satellite cells and or myoblasts. Although Myf5 is expressed in the majority of myogenic cells, myogenin is expressed by differentiating myoblasts. The number of Myf5+ nuclei was slightly higher but not significantly different with S1P treatment (FIGS. 19a and b). These results indicate the increased number of the Myf5+ cells observed with S1P treatment are mainly activated satellite cells and not differentiating myoblasts.

It was then determined whether the increase in myogenic cells promotes dystrophic muscle repair by staining for embryonic Myosin Heavy Chain (eMyHC), a marker for regenerating muscle fibers. In concurrence with the rise of Myf5+ myogenic cells, a 3.6-fold increase in the number of eMyHC+ fibers was observed in S1P treated TAs (FIG. 5e,f). Furthermore, the size of regenerating myofibers in S1P treated TAs was significantly greater, as indicated by the minimum diameter quantified for the largest eMyHC+ fibers (FIG. 5g). Therefore, administration of S1P promotes dystrophic muscle regeneration by improving satellite cell response and subsequent muscle fiber regeneration.

### Example 6

### S1P Administration Correlates with Increased Levels of Phosphorylated Ribosomal S6, an Indicator of Protein Synthesis

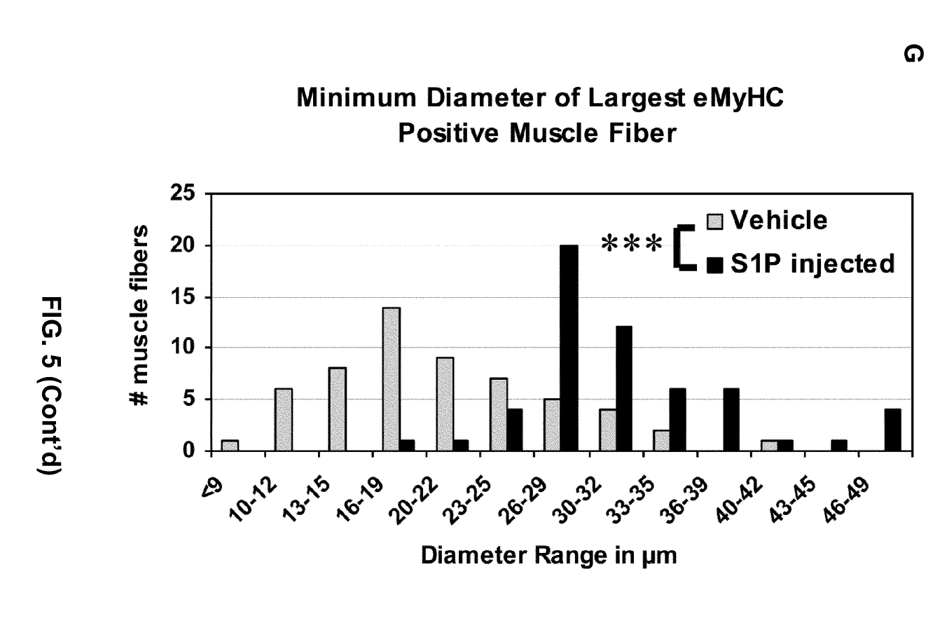

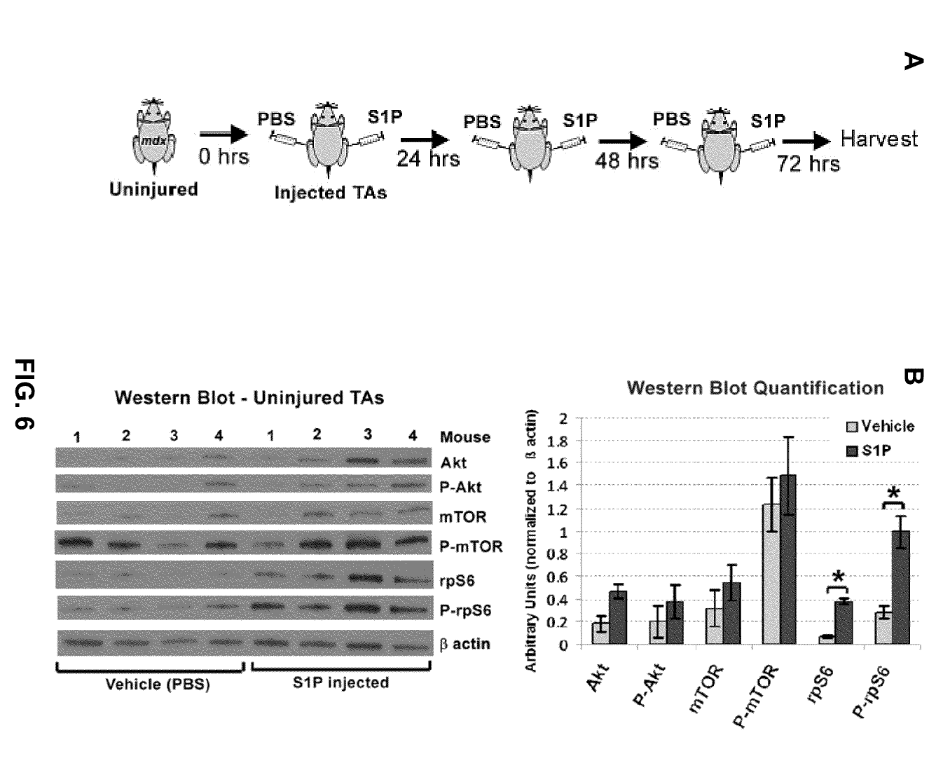

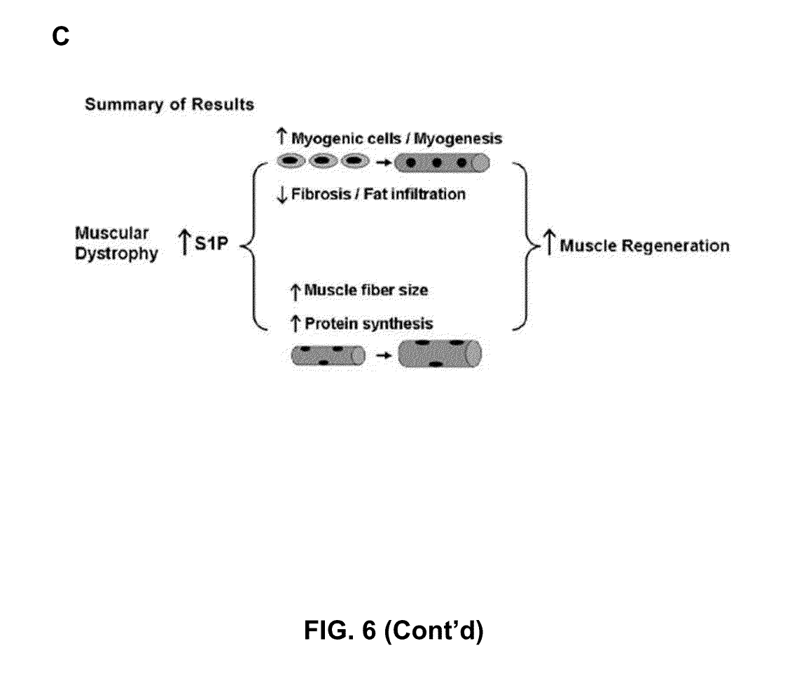

To discern if the S1P dependent increase in mdx muscle fiber size was due to hypertrophy, the activation of Akt/mTOR signaling was examined. S1P induced hypertrophy has been described in cultured cardiomyocytes, which was accompanied by activation of Akt and S6 kinase. In turn, Akt and mTOR signaling via S6 kinase, an activator of ribosomal S6 implicated in protein synthesis, has been described as sufficient to induce skeletal muscle hypertrophy. Therefore it was determined if the increase in muscle fibers by S1P was mediated by Akt/mTOR signaling. Since THI induces a S1P increase throughout the body, S1P was administered directly into muscle in mdx mice and levels of activated/phosphorylated Akt, mTOR, and S6 were measured. S1P was injected directly into uninjured TA muscle to measure potential effects on these pathways in the absence of injury. As before, S1P was injected into left TAs and vehicle into right TAs of the same animals (FIG. 6a). Following three days of daily injections, muscles were harvested for protein analysis by western blot. The data show that the levels of phosphorylated Akt and mTOR, though increased, were not significantly higher in S1P treated muscles (FIG. 6b). However, the levels of S6 and phosphorylated S6 were significantly increased with S1P treatment compared to control muscles indicating an increase in protein synthesis (FIG. 6b). These data suggest that S1P also upregulates anabolic pathways in the mdx mouse. S1P may drive skeletal muscle protein synthesis directly through S6 and phospho-S6 upregulation or via another pathway in addition to Akt/mTOR signaling.

### Example 7

### S1P Administration in a Dysferlinopathy Model

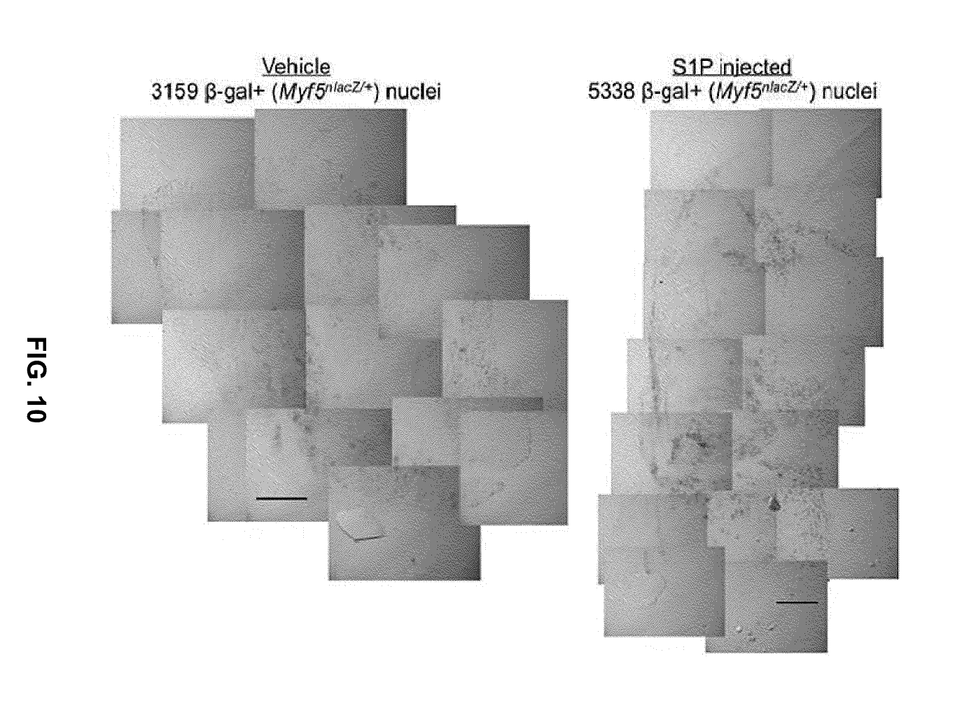

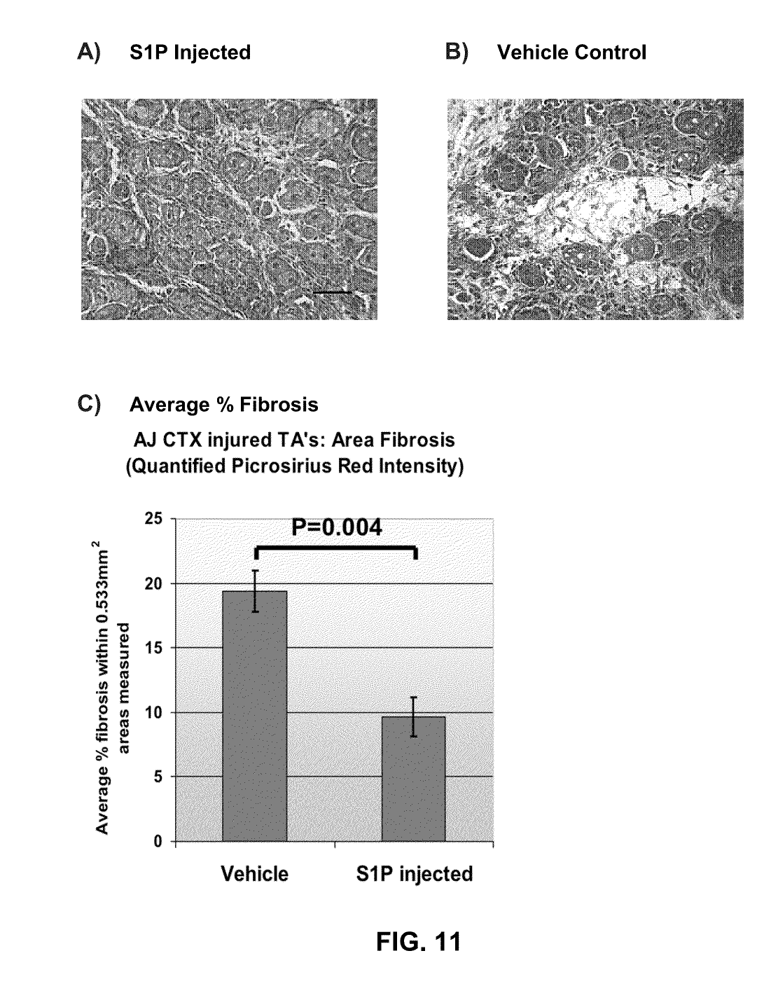

The A/J mouse strain (JAX Mice, Bar Habor, Me.) lacks dysferlin and is a model for dysferlinopathy. The effects of S1P treatment of A/J mice was examined following CTX induced acute injury. CTX was applied to both TA muscles (2× male, 2× female, 9 month old), then S1P was injected intramuscularly into left TAs and a vehicle control (saline) in right TAs daily for the first three days following CTX injury. The TAs were harvested and examined for muscle fibrosis (picrosirius red staining) 6 days post injury. The S1P treated muscles showed a decrease in muscle fibrosis as compared to vehicle controls (FIGS. 11a and 11b). Furthermore, S1P treatment of CTX injured TA muscles in the A/J mice significantly decreased the average percentage of fibrosis (FIG. 11c).

### Example 8

### Improved Muscle Recovery and Function in Dystrophic Mice Treated with THI

The effect of THI administration on muscle function and recovery in mdx mice was analyzed. The mice were injected intraperitoneally with THI in PBS or vehicle alone (PBS) twice-daily for two weeks. Total amount of THI delivered per day was 75 ug. Extensor digitorum longus (EDL) muscles were excised from the mice and equilibrated in ringer's solution with 95% O₂/5% CO₂ for a minimum of 15 minutes prior to stimulation. Myography was conducted using a DMT 820S myograph and data was recorded using PowerLab 4/30 acquisition system with LabChart Pro software v7.3.1 (both from ADI instruments, Colorado Springs, Colo.). Stimulations were conducted with a Grass Instrument S88X system (Grass Technologies, West Warwick, R.I.). Muscles were stimulated to establish optimal fiber length (Lf) and voltage at which maximum tetanic force was measured at 120 Hz within a 450 ms duration.

Specific force was calculated as previously described in Gregorevic P. et al. (Gregorevic P. et al., Muscle Nerve. 2004 September; 30(3):295-304, PMID#15318340) by normalizing to whole muscle cross-sectional area (CSA). CSA is the quotient of dry muscle mass over the optimal muscle length (Lₒ) which is defined as the product of muscle fiber length (L_{f}) with the fiber length ratio (0.44 for EDL) and mammalian muscle density (1.06).

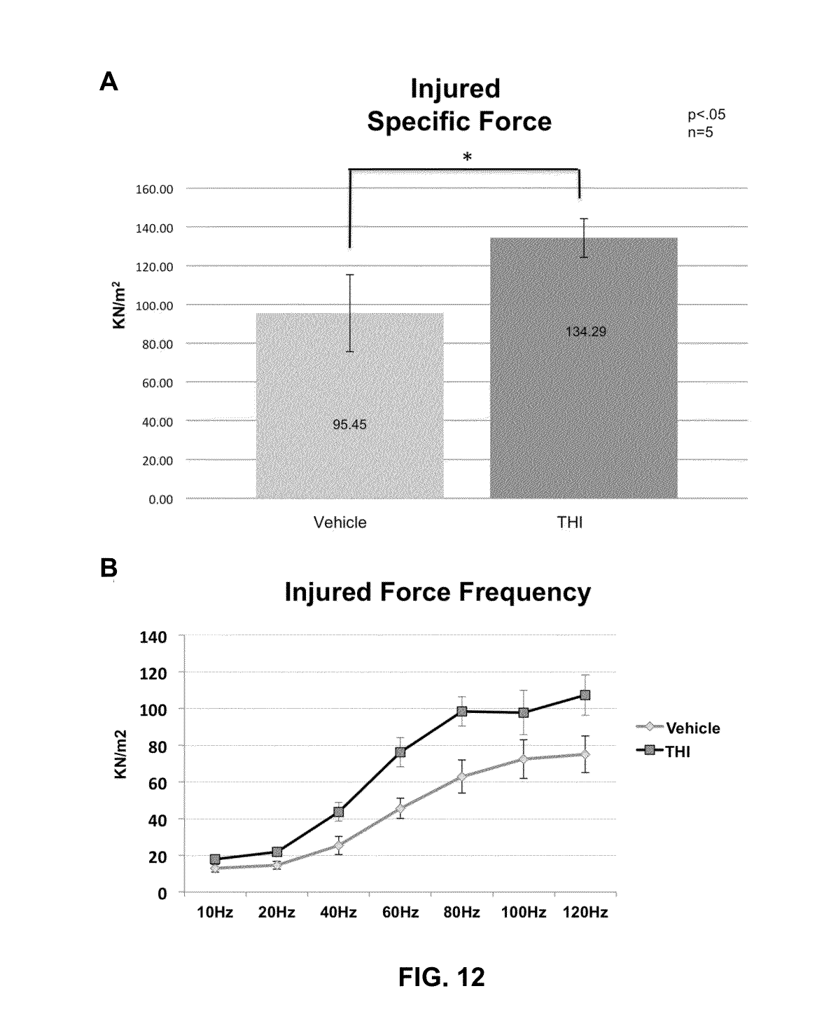

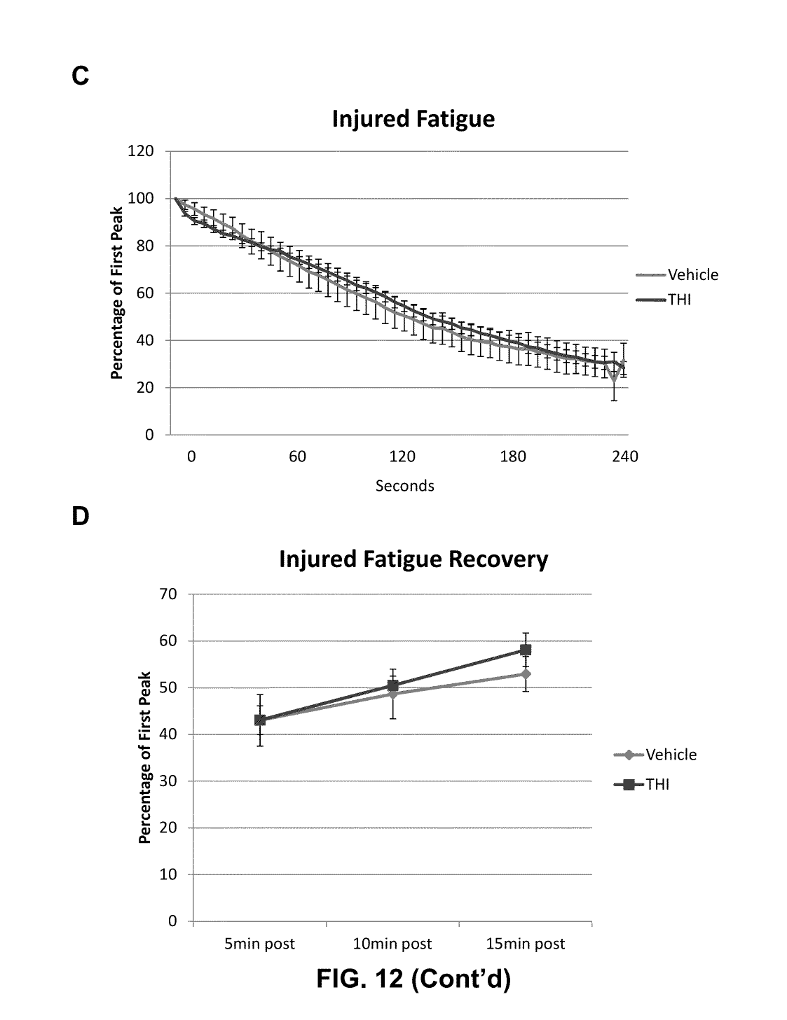

The specific force of the muscle was measured as the maximum force (FIG. 12a) and the force measured at elevating frequency (FIG. 12b). A maximum specific force of 95.45 KN/m² was measured for vehicle alone and 134.29 KN/m² was measured for THI treated muscle (FIG. 12a), showing a statistically significant increase in specific force of THI treated muscle. FIG. 12b indicates the elevation of specific force stimulated at elevating frequencies for THI-treated muscle and vehicle-treated muscle.

Muscle recovery was measured after muscle was fatigued with stimulation over 6 minutes. The vehicle-treated muscles and the THI-treated muscles fatigued at similar rates (FIG. 12c). Recovery was determined by examining muscle force at 5, 10, and 15 minutes after stimulation. There was an improved recovery in THI-treated muscles compared to the vehicle-treated muscles (FIG. 12d). Therefore, the administration of THI improved muscle recovery and function in dystrophic mice.

### Example 9

### Suppression of Muscle Wasting after Administration of S1P Promoting Agents THI, a Derivative of THI, and a Sphingosine Analog

The effect of THI, a derivative of THI ((E)-1-(4-((1R,2S,3R)-1,2,3,4-tetrahydroxybutyl)-1H-imidazol-2-yl)-ethanone oxime) (“THI-oxime”), and a sphingosine analog (FTY720) on muscle wasting in dystrophic Drosopila flies was observed. Before feeding, 4 day old dystrophic flies (genotype: w; +/+; Dys^{det1}/Dys^{det1}) were placed in empty vials and starved for 6 hours. The flies were then provided access to one of a solution of 0.05 mg/mL (217 μM) 2-Acetyl-4(5)-tetrahydroxybutyl Imidazole (THI), or 217 μM FTY720, or 217 μM THI-oxime, in 5% light corn syrup in water for 16-18 hrs. After feeding, the flies were placed in new yeasted vials for 6-7 hrs. Flies were then transferred to empty vials with access to a 217 μM solution of THI, FTY720 or THI-oxime in a 5% light corn syrup in water for 16-18 hrs. This feeding cycle was repeated for a third time. After feeding for three cycles, the flies were placed in new yeasted vials. The 7-day old flies that had been administered the THI-oxime were analyzed immediately after the last feeding. The flies that has been administered THI and FTY720 were analyzed as 10-day old flies, three days after the last feeding with THI and FTY720.

Myofibril immunochemistry analysis was performed as disclosed herein. For each treatment group, 5 flies were analyzed, and 3 muscles per fly and 2 myofibrils per muscle were analyzed. Two tailed Student's t test was used to evaluate significance.

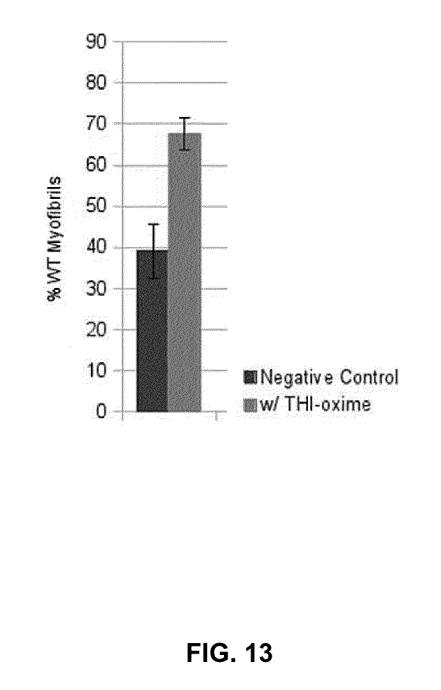

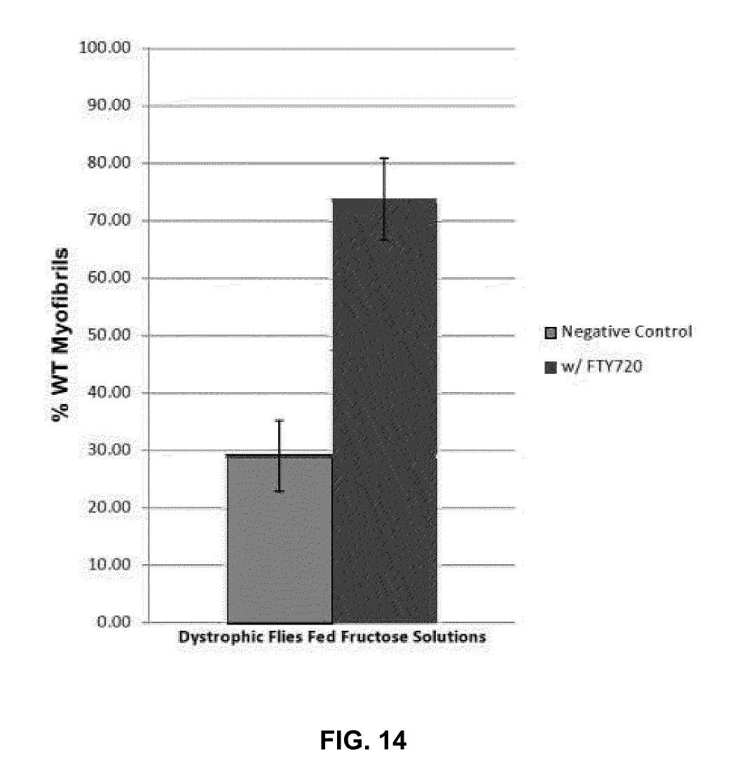

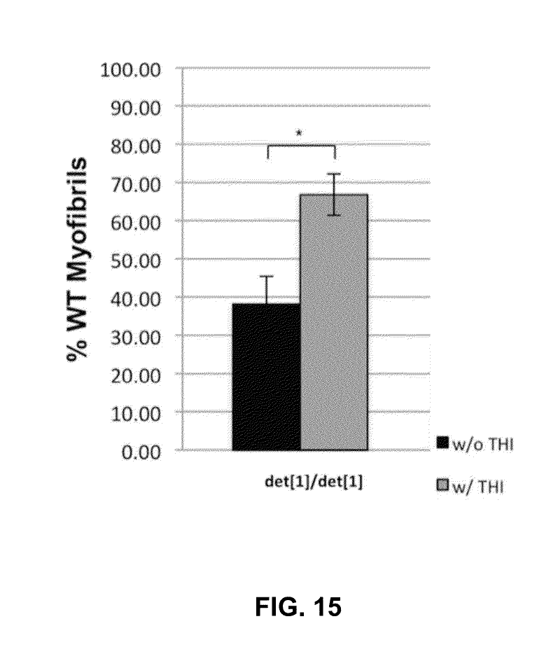

As shown in FIG. 13, the muscles sampled from the 7-day old flies administered THI-oxime demonstrate a significant suppression of muscle wasting having 67.62% wild-type myofibrils compared to 39.05% wild-type myofibrils in the negative control (p=0.0236). FIG. 14 shows that the muscles from the 10-day old flies that were administered FTY720 also reveal a significant suppression of muscle wasting having 73.83% wild-type myofibrils compared to only 29.05% wild-type myofibrils in the negative control (p=0.0021). Administration of THI also shown a significant suppression of muscle wasting in 10-day old flies with approximately 67% wild type myofibrils in the THI treated flies versus approximately only 38% wild type myofibrils in negative control (p<0.05, FIG. 15).

Therefore, administration of THI, a derivative of THI, and a sphingosine analog suppress muscle wasting in dystrophic flies.

### Example 10

### Improved Muscle Function in Dystrophic Mice Treated with THI-Oxime

The effect of THI-oxime administration on muscle function and recovery in mdx mice was analyzed. The mice were injected intraperitoneally with THI-oxime (n=3) or PBS vehicle alone (n=4) twice-daily for two weeks and then analyzed between 1-4 days following the final day of injection. Total amount of THI-oxime delivered per day was 75 ug. Prior to euthanasia, the animals were anesthetized with 0.5 mg/g weight avertin diluted in PBS. EDL muscles were excised from the mice and equilibrated in ringer's solution (120 mM NaCl, 4.7 mM KCl, 3.15 mM MgCl₂, 1.3 mM NaH₂PO₄, 25 mM NaHCO₃, 11 mM Glucose, 1.25 mM CaCl₂, pH 7.2) with 95% O₂/5% CO₂ for a minimum of 15 minutes prior to stimulation. All functional experiments were carried out with buffer solutions at 25° C. under constant oxygenation. Myography was conducted using a DMT 820S myograph and data was recorded using PowerLab 4/30 acquisition system with LabChart Pro software v7.3.1 (both from ADI instruments, Colorado Springs, Colo.). Stimulations were conducted with a Grass Instrument S88X system (Grass Technologies, West Warwick, R.I.). Muscles were stimulated to establish optimal fiber length (Lf) and voltage at which maximum tetanic force was measured at 120 Hz within a 450 ms duration. Force frequency was carried out using the same pulse duration at 10, 20, 40, 60, 80, 100, and 120 Hz.

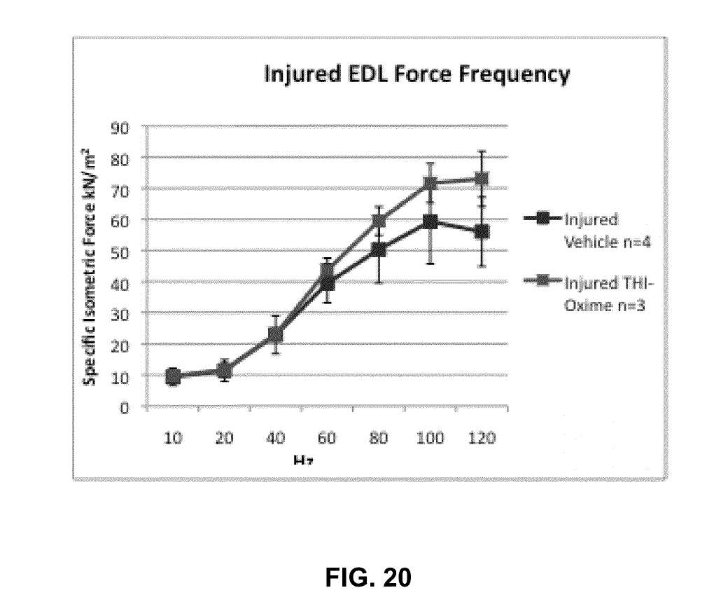

Force frequency was calculated as in Example 8. As shown in FIG. 20, the specific force for the injured EDLs was measured during stimulation at elevating frequencies for THI-oxime-treated muscle and vehicle-treated muscle. A maximum specific force of 58 KN/m², at 100 Hz, was measured for vehicle controls alone and 73 KN/m², at 120 Hz, was measured for THI-oxime treated muscle. The results show that the administration of THI-oxime improved muscle function in dystrophic mice.

Materials & Methods

Fly Stocks—

The fly strains used in this study were: Oregon-R, w¹¹¹⁸, Dys^{det1}, wun^{EMS4}/CyO, tubGal4/TM3, cn¹, Sply⁰⁵⁰⁹¹/CyO; ry⁵⁰⁶, Df(3R)Exel6184/TM6B (outcrossed to w¹¹¹⁸ at least seven times) obtained from the Bloomington Stock Center. UAS-HA-lace/CyO kindly provided by Dr. H. Date. wun^{k10201}/CyO (y[d2] w[1118] P{ry[+t7.2]=ey-FLP.N}2 P{GMR-lacZ.C(38.1)}TPN1; P{ry[+t7.2]=neoFRT}42D P{w[+mC]=lacW}wun[k10201] I(2)k10201[k10201]P{lacW}k10201b/CyO y[+] (Stock #111469)) obtained from the Kyoto Drosophila Genetic Resource Center. wunen^{RNAi} ([I(2)k16806]/TM3 (stock #6446)) obtained from the Vienna Drosophila RNAi Center. ActGal4:UAS-Dys^{N-RNAi}/CyO and TubGal4:UAS-Dys^{C-RNAi}/TM6B recombinant lines were generated in this lab.

Activity Assay—

The TriKinetics, Inc. DAM5 activity monitoring system was used to record the movements of individual flies over the course of different lengths of time. Eight flies were monitored for each genotype per experiment. Data for the two least active flies for each genotype were eliminated leaving data for the 6 most active flies per experiment. Each experiment was repeated 3 times.

Myofibril Immunohistochemistry—

Flies were dipped in 95% EtOH and then dissected in 1×PBS (pH 7.4). Heads and abdomens were removed, and the thoraxes opened. Samples were stained using a modified ovary staining protocol 3. Flies were dipped in 95% EtOH and then dissected in 1×PBS (pH 7.4). Heads and abdomens were removed, and the thoraxes opened. Samples were then fixed in 5% paraformaldehyde (Electron Microscopy Sciences) for 1 hour, rinsed in PBT (PBS/0.2% Triton X-100 v/v) 4 times, and then blocked for 1 hour in PBTB (PBT, 0.4% BSA w/v, 5% Normal Goat Serum v/v) at room temperature. Samples were stained in primary antibody pre-diluted in PBTB (Phalloidin-Alexa-Fluor 568, Invitrogen [mouse, 1:200]; Projectin (Mac150), Babraham Institute [rat, 1:50]) overnight at 4° C., rinsed in PBT 4 times at room temperature (RT), and then stained with secondary antibody pre-diluted in PBTB (Alexa Fluor 488 [mouse, 1:500]) overnight at 4° C. Samples were then rinsed in PBT 4 times at RT and stored in 80% glycerol/3% n-propyl gallate w/v, /20% Prolong Gold (Invitrogen) v/v. Analysis was performed using a Leica TCS-SPE Confocal microscope with a 40× objective and Leica Software. For each genotype, 4-5 flies were analyzed, and 6-7 muscles per fly were analyzed.

Oral Administration of THI (Flies)—

For oral administration of THI, 4 day-old flies were place in empty vials and starved for 6 hours. For feeding of THI, the flies were then provided access to a solution of 0.05 mg/mL THI in a 5% fructose PBS solution for 16 hrs. The flies were placed in new yeasted vials for 6-7 hrs. Flies were then transferred to empty vials with access to a solution of 0.05 mg/mL THI in a 5% fructose PBS solution for 18 hrs. The flies were placed in new yeasted vials for 6-7 hrs. Flies were then transferred to empty vials with access to a solution of 0.05 mg/mL THI in a 5% fructose PBS solution for 18 hrs. The flies were placed in new yeasted vials. The flies were then transferred to new yeasted vials daily for three more days before dissection.

Creatine Kinase Assay—

Mdx mouse plasma samples were diluted 1:50 and total creatine kinsase activity was measured by an enzymatic rate method using a Beckman Coulter instrument. Relative levels were then normalized to body weight.

S1P Infections—

Right and left TAs of three, 3 month old male mdx4cv:Myf5nlacZ/+ were injected with 10 mM cardiotoxin (Calbiochem) in PBS from Naja nigrcollis. For the dysferlinopathy study, right and left TAs of 9 month old male and female A/J mice (JAX Mice, Bar Harbor, Me.) were injected with 10 mM cardiotoxin (Calbiochem) in PBS from Naja nigrcollis. S1P (Enzo; Calbiochem) preparation was done according to manufacturer's instructions. Briefly, S1P was dissolved in methanol (0.5 mg/ml) and aliquoted, then the solvent was evaporated with a stream of nitrogen to deposit a thin film on the inside of the tube. Aliquots were dissolved with 4 mg/ml BSA (fatty acid free) to make 500 uM stocks. Directly following cardiotoxin injection, 500 uM S1P in PBS with 4 mg/ml BSA was injected in left TAs (20 ul), daily until day 3 post-injury at which time animals were euthanized and muscles were harvested for freezing. Right TAs were injected with an equal volume of PBS with 4 mg/ml BSA as vehicle controls. In a separate experiment TAs of four 2.5 month-old female mdx4cv were injected with S1P or vehicle under the same conditions stated above, in the absence of injury.

THI Injections (Mice)—

The left TAs and quadriceps of six, 10 month old female mdx4cv mice were injected with 50 μl and 100 μl CTX respectively. Three mice were injected IP with 0.15 mg/ml THI in PBS, twice daily (injections 8 hours apart) immediately after and for the first three days following injury. The three remaining animals were injected IP with PBS as vehicle controls. 1% Evans Blue dye was injected IV on day 17 post CTX, to label persistently damaged (dye permeable) muscle fibers. Animals were euthanized 18 days post injury and muscles were frozen under liquid nitrogen cooled isopentane in optimal cutting temperature (OCT). All myofibers were measured for the minimum diameters on the cross-sections of mouse quadriceps muscle using image J software. 750˜850 myofibers were counted for three mice treated with PBS or THI with or without CTX injury.

Mouse Histology and Staining—

All mouse muscles were frozen directly in OCT with liquid nitrogen cooled in isopentane and sections of 8 μm thickness were obtained. Tissue for x-gal staining, was fixed for 10 min with 2% formaldehyde/0.2% glutaraldehyde and incubated overnight at 37° C. with staining buffer (PBS with 1 mg/ml x-gal, 5 mM potassium ferricyanide, 5 mM potassium ferrocyanide, and 2 mM CaCl₂ (all from Fisher). Oil Red-O and Picrosirius red staining were done following established protocols. For eMyHC staining, tissue was first fixed with 2% formaldehyde for 5 min, treated with streptavidin/avidin blocking kit (Vector labs), and blocked with IgG block from M.O.M kit (Vector Labs) for 5 hrs at 4° C. Following blockade, concentrated mouse anti-eMyHC (clone F1.652 received at 357 μg/ml IgG) obtained from the developmental studies hybridoma bank (DHSB, University of Iowa) was administered at 1:400 dilution, overnight at 4° C. The remainder of the staining was done following mouse-on-mouse (M.O.M) kit staining instructions. For laminin staining tissue was also fixed with 2% formaldehyde for 5 min, and then treated with polyclonal rabbit anti-laminin (Sigma) for 1 hr at a 1:400 dilution in PBS+1% BSA. Following washes, Alexa Fluor 488 conjugated goat anti-rabbit IgG (Invitrogen) was administered at 1:800 dilution for 1 hr. Controls omitting the primary antibody were included with all staining. Staining quantifications were all done using ImageJ v1.40 (Wayne Rasband, NIH) cell counter plugin (Kurt De Vos, University of Sheffield). Calculations, statistics, and graphs were generated with Microsoft Excel. Brightfield photographs were captured using either a Fisher Micromaster digital inverted or upright microscopes with Micron software. Fluorescent photographs were captured with a monochromatic camera using a Zeiss Axiovert 200 microscope. Individual fluorescent channels were colored and merged using Adobe Photoshop CS2. Brightness contrast levels were adjusted to increase visibility and reduce background in most photographs.

Western Blot Analysis—

Tissue for western blot analysis was snap frozen in liquid nitrogen and subsequently homogenized. Freshly isolated TA muscles were harvested and snap frozen in liquid nitrogen prior to homogenization with disposable tissue grinders. Tissue was homogenized under liquid nitrogen then resuspended in lysis buffer containing 50 mM Tris HCl (pH 7.4), 1 mM ethylenediaminetetraacetic acid (EDTA), 150 mM NaCl, 5 mM NaF, 0.25% (w/v) Na deoxycholate, 2 mM NaVO3, 1% Triton X-100 (v/v), supplemented with complete protease inhibitor cocktail from Roche and complete phosphatase inhibitor cocktails 1 and 2 from Sigma. Protein extracts were separated using BioRad Tris-HCl ready gels 4-20% linear gradient and transferred to polyvinylidene difluoride (PVDF) membranes with a wet transfer system (BioRad). Membranes were blocked for 1 h with Tris buffered saline with 0.1% (v/v) Tween 20 containing 5% (w/v) BSA. Polyclonal antibodies were used to blot against phosphorylated (Thr308) Akt, Akt, phosphorylated (Ser2448) mTOR, mTOR, phosphorylated (Ser240/Ser244) S6 ribosomal protein, S6 ribosomal protein, and β-actin (Cell Signaling). The signals were detected using an enhanced chemiluminescence kit (Millipore) and CL-XPosure Films (Thermo Scientific) were analyzed using ImageJ.

Statistics—

Two tailed Student's t-test was used to determine statistical significance for all experiments.

Climbing Assay—

An apparatus made for fractionating a Drosophila population using a countercurrent distribution procedure was used to measure climbing ability of different fly cohorts. The apparatus was kindly loaned from Leo Pallanck. 20-30 flies were tested for their ability to climb at least 10 cm in 30 seconds for five consecutive times. Each group of flies was tested for 5 trials with 3 minutes separating each trial. Each set of five trials was then given a Climbing Index (CI) value. The CI is a weighted average of the number of flies that end up in progressively more distant tubes from the first tube of the apparatus. Weighted values were 0, 2, 4, 8, 16, and 32 from the first to the last tube, respectively. Each experiment was repeated 3 times.

Indirect Flight Muscle Histology—

A Histological sections of IFMs were prepared from paraffin wax embedded material. Briefly, flies were immobilized in Heisenberg fly collars (Model #10731, 4M Instrument & Tool LLC, New York) between the abdomen and thorax, then fixed in Carnoy's solution (6:3:1 ethanol (EtOH):chloroform:glacial acetic acid) overnight at 4° C. After fixation, they were hydrated/dehydrated to remove the Carnoy's with the following procedure: 40% EtOH (1×10′), 75% EtOH (1×10′), 95% EtOH (1×10′), then 100% EtOH (2×10′). All performed at room temperature (RT). The flies were then infiltrated with paraffin (Poly/Fin, Triangle Biomedical Sciences, Inc) using the following procedure: Samples were placed in methyl benzoate for 30 min at 65° C., then transferred to a methyl benzoate:paraffin solution (1:1) and further incubated at 65° C. for 30 min, and finally placed in paraffin alone at 65° C. for an additional 30 min. Afterwards samples were placed in casts then filled with melted paraffin (65° C.). Once cooled and solidified, the collars were removed, which sheared off the fly abdomens. Transverse sections of the fly thoraces were cut with a rotary microtome (Leica 820 Histocut) at 10 um thickness per section. Paraffin was removed with xylene (2×4′). The sections were then rehydrated (100% EtOH 2×4, 95% EtOH 1×3′, 70% EtOH 1×2′, H₂O 1×1′), and then stained with hematoxylin and eosin using standard protocols. Sections were covered with DPX Mountant (Fluka), cover-slipped and analyzed using light microscopy (Leica) with the 20× objective. The middle third (15-20 sections) of the thorax, which was cut in sections of 10 um thickness, of each fly was scored for degeneration. Each section was rated A thru E depending on the severity of the fragmentation phenotype. A) >2 of the IFMs were unfragmented, B) at least 1 IFM was unfragmented, C) fragmentation in all IFMs. D) severe degeneration in at least 1 IFM. E) severe degeneration in >1 IFMs. A Muscle Integrity Index (MII) was then calculated. The MII is a weighted average of sections showing less degeneration over time (A=16, B=8, C=4, D=2, and E=0). Each fly was given an overall score from ˜20 sections. 8-12 flies were scored for each experiment, each experiment was repeated 3 times.

Pax7 and Myogenin Stainings—

Both stainings were done using freshly frozen mdx muscles. Pax7 staining was done as outlined by Clever et al., with slight modification (Clever J L, et al. Am J Physiol Cell Physiol. 2010; 298:C1087-C1099). Sections were fixed overnight in 4% formaldehyde (from PFA powder) at 4° C. Following fixation, antigen retrieval was done with 10 mM citrate buffer (with 0.05% Teen20 at pH 6.0) warmed in a water bath at 90° C. for 20 minutes. Slides were then permeated with ice cold methanol for 5 minutes at room temperature. Streptavidin/Biotin blocking (Vector labs) was done according to manufacturer's instructions. Staining was done using the M.O.M. kit (Vector labs) with IgG blocking for 5 hours at 4° C. prior to addition of mouse monoclonal anti-Pax7 (clone PAX7, R&D) diluted at 1:20 and incubated overnight at 4° C. Biotinylated anti-mouse secondary was supplied by and used as prescribed by M.O.M kit instructions. Streptavidin conjugated to Alexa Fluor 488 (Invitrogen) was added at 1:1000. As a negative control for Pax7 staining, mouse IgG isotype was applied to separate ribbons and treated in parallel. For myogenin staining, tissues were fixed with ice cold methanol for 5 minutes at room temperature then blocked with 10% Horse Serum and 1% BSA for 30 minutes. Monoclonal mouse anti-myogenin directly conjugated to AlexaFluor 488 (clone F5D, eBioscience) was applied at 1:50 for 1 hr at room temperature.

It will be obvious to those having skill in the art that many changes may be made to the details of the above-described embodiments without departing from the underlying principles of the invention. The scope of the present invention should, therefore, be determined only by the following claims.

1. A method of treating a degenerative muscle condition in a subject, the method comprising administering a therapeutically effective amount of an S1P promoting composition comprising an S1P promoting agent.

2. The method of claim 1, wherein administering a therapeutically effective amount of an S1P promoting composition comprises administering a therapeutically effective amount of an S1P promoting composition comprising an S1P promoting agent selected from THI, biologically active derivatives of THI, and pharmaceutically acceptable salts, esters, isomers and solvates thereof.

3. The method of claim 1, wherein administering a therapeutically effective amount of an S1P promoting composition comprises administering a therapeutically effective amount of an S1P promoting composition comprising an S1P promoting agent selected from biologically active derivatives of THI and pharmaceutically acceptable salts, esters, isomers and solvates thereof.

4. The method of claim 1, wherein administering a therapeutically effective amount of an S1P promoting composition comprises administering a therapeutically effective amount of an S1P promoting composition comprising an S1P promoting agent selected from THI and pharmaceutically acceptable salts, esters, isomers and solvates thereof.

5. The method of claim 1, wherein administering a therapeutically effective amount of an S1P promoting composition comprises administering a therapeutically effective amount of an S1P promoting composition comprising S1P and pharmaceutically acceptable salts, esters, isomers and solvates thereof.

6. The method of claim 1, wherein administering a therapeutically effective amount of an S1P promoting composition comprises administering a therapeutically effective amount of an S1P promoting composition comprising a sphingosine analog and pharmaceutically acceptable salts, esters, isomers and solvates thereof.

7. The method of claim 1, wherein the S1P promoting agent comprises a biologically active derivative of THI according to Formula III: 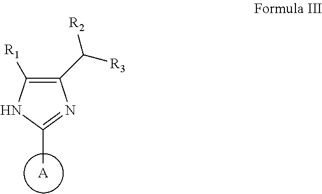
and pharmaceutically acceptable salts, esters, isomers and solvates thereof, wherein:
A is an optionally substituted heterocycle;
R₁ is OR_{1A}, OC(O)R_{1A}, C(O)OR_{1A}, hydrogen, halogen, nitrile, or optionally substituted alkyl, aryl, alkylaryl, arylalkyl, heteroalkyl, heterocycle, alkylheterocycle, or heterocyclealkyl;
R₂ is OR_{2A}, OC(O)R_{2A}, hydrogen, halogen, or optionally substituted alkyl, aryl, alkylaryl, arylalkyl, heteroalkyl, heterocycle, alkylheterocycle, or heterocyclealkyl;
R₃ is N(R_{3A})₂, hydrogen, hydroxy, or optionally substituted alkyl, aryl, alkylaryl, arylalkyl, heteroalkyl, heterocycle, alkylheterocycle, or heterocyclealkyl; and
each of R_{1A}, R_{2A}, and R_{3A} is independently hydrogen or optionally substituted alkyl, aryl, alkylaryl, arylalkyl, heteroalkyl, heterocycle, alkylheterocycle, or heterocyclealkyl.

8. A method according to claim 1, wherein the S1P promoting agent comprises a biologically active derivative of THI according to Formula IV: 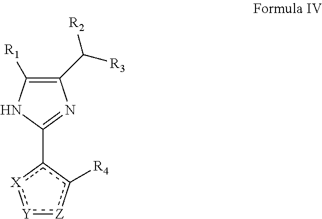
and pharmaceutically acceptable salts, esters, isomers and solvates thereof, wherein:
X is CR₄, CHR₄, N, NR₉, O or S; Y is CR₄, CHR₄, N, NR₉, O or S; Z is CR₄, CHR₄, N, NR₉, O or S;
R₁ is OR_{1A}, C(O)OR_{1A}, hydrogen, halogen, nitrile, or optionally substituted alkyl, aryl, alkylaryl, arylalkyl, heteroalkyl, heterocycle, alkylheterocycle, or heterocyclealkyl;
R₂ is OR_{2A}, OC(O)R_{2A}, hydrogen, halogen, or optionally substituted alkyl, aryl, alkylaryl, arylalkyl, heteroalkyl, heterocycle, alkylheterocycle, or heterocyclealkyl;
R₃ is N(R_{3A})₂, hydrogen, hydroxy, or optionally substituted alkyl, aryl, alkylaryl, arylalkyl, heteroalkyl, heterocycle, alkylheterocycle, or heterocyclealkyl;
R₄ is OR_{4A}, OC(O)R_{4A}, hydrogen, halogen, or optionally substituted alkyl, aryl, alkylaryl, arylalkyl, heteroalkyl, heterocycle, alkylheterocycle, or heterocyclealkyl;
each R₉ is independently hydrogen or optionally substituted alkyl, aryl, alkylaryl, arylalkyl, heteroalkyl, heterocycle, alkylheterocycle, or heterocyclealkyl; and
each of R_{1A}, R_{2A}, R_{3A} and R_{4A} is independently hydrogen or optionally substituted alkyl, aryl, alkylaryl, arylalkyl, heteroalkyl, heterocycle, alkylheterocycle, or heterocyclealkyl.

9. The method of claim 1, wherein the S1P promoting agent comprises a biologically active derivative of THI according to Formula V: 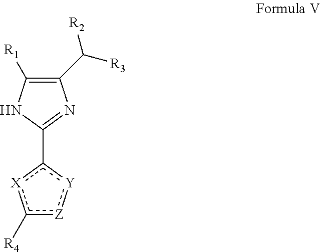
and pharmaceutically acceptable salts, esters, isomers and solvates thereof, wherein:
X is CR₄, CHR₄, N, NR₉, O or S;
Y is CR₄, CHR₄, N, NR₉, O or S;
Z is CR₄, CHR₄, N, NR₉, O or S;
R₁ is OR_{1A}, C(O)OR_{1A}, hydrogen, halogen, nitrile, or optionally substituted alkyl, aryl, alkylaryl, arylalkyl, heteroalkyl, heterocycle, alkylheterocycle, or heterocyclealkyl;
R₂ is OR_{2A}, OC(O)R_{2A}, hydrogen, halogen, or optionally substituted alkyl, aryl, alkylaryl, arylalkyl, heteroalkyl, heterocycle, alkylheterocycle, or heterocyclealkyl;
R₃ is N(R_{3A})₂, hydrogen, hydroxy, or optionally substituted alkyl, aryl, alkylaryl, arylalkyl, heteroalkyl, heterocycle, alkylheterocycle, or heterocyclealkyl;
R₄ is independently OR_{4A}, OC(O)R_{4A}, hydrogen, halogen, or optionally substituted alkyl, aryl, alkylaryl, arylalkyl, heteroalkyl, heterocycle, alkylheterocycle, or heterocyclealkyl;
each R₉ is independently hydrogen or optionally substituted alkyl, aryl, alkylaryl, arylalkyl, heteroalkyl, heterocycle, alkylheterocycle, or heterocyclealkyl; and
each of R_{1A}, R_{2A}, R_{3A} and R_{4A} is independently hydrogen or optionally substituted alkyl, aryl, alkylaryl, arylalkyl, heteroalkyl, heterocycle, alkylheterocycle, or heterocyclealkyl.

10. The method of claim 1, wherein the S1P promoting agent comprises a biologically active derivative of THI according to Formula VI: 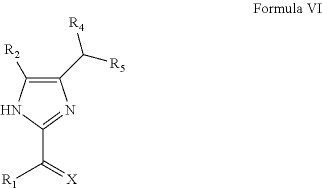
and pharmaceutically acceptable salts, esters, isomers and solvates thereof, wherein:
X is O or NR₃;
R₁ is OR_{1A}, NHOH, hydrogen, or optionally substituted alkyl, aryl, alkylaryl, arylalkyl, heteroalkyl, heterocycle, alkylheterocycle, or heterocyclealkyl;
R₂ is OR_{2A}, C(O)OR_{2A}, hydrogen, halogen, nitrile, or optionally substituted alkyl, aryl, alkylaryl, arylalkyl, heteroalkyl, heterocycle, alkylheterocycle, or heterocyclealkyl;
R₃ is OR_{3A}, N(R_{3A})₂, NHC(O)R_{3A}, NHSO₂R_{3A}, or hydrogen;
R₄ is OR_{4A}, OC(O)R_{4A}, hydrogen, halogen, or optionally substituted alkyl, aryl, alkylaryl, arylalkyl, heteroalkyl, heterocycle, alkylheterocycle, or heterocyclealkyl;
R₅ is N(R_{5A})₂, hydrogen, hydroxy, or optionally substituted alkyl, aryl, alkylaryl, arylalkyl, heteroalkyl, heterocycle, alkylheterocycle, or heterocyclealkyl; and
each of R_{1A}, R_{2A}, R_{3A}, R_{4A}, and R_{5A} is independently hydrogen or optionally substituted alkyl, aryl, alkylaryl, arylalkyl, heteroalkyl, heterocycle, alkylheterocycle, or heterocyclealkyl.

11. The method of claim 1, wherein the S1P promoting agent comprises a biologically active derivative of THI according to Formula VII: 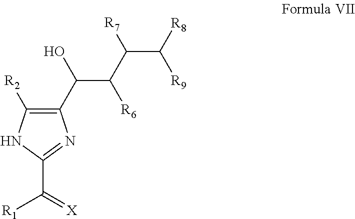
and pharmaceutically acceptable salts, esters, isomers and solvates thereof, wherein:
X is O or NR₃; R₁ is OR_{1A}, NHOH, hydrogen, or optionally substituted alkyl, aryl, alkylaryl, arylalkyl, heteroalkyl, heterocycle, alkylheterocycle, or heterocyclealkyl;
R₂ is OR_{2A}, C(O)OR_{2A}, hydrogen, halogen, nitrile, or optionally substituted alkyl, aryl, alkylaryl, arylalkyl, heteroalkyl, heterocycle, alkylheterocycle, or heterocyclealkyl;
R₃ is OR_{3A}, N(R_{3A})₂, NHC(O)R_{3A}, NHSO₂R_{3A}, or hydrogen;
R₆ is OR_{6A}, OC(O)R_{6A}, N(R_{6B})₂, NHC(O)R_{6B}, hydrogen, or halogen;
R₇ is OR_{7A}, OC(O)R_{7A}, N(R_{7B})₂, NHC(O)R_{7B}, hydrogen, or halogen;
R₈ is OR_{gA}, OC(O)R_{BA}, N(R_{8B})₂, NHC(O)R_{8B}, hydrogen, or halogen;
R₉ is CH₂OR_{9A}, CH₂OC(O)R_{9A}, N(R_{9B})₂, NHC(O)R_{9B}, hydrogen, or halogen;
each of R_{1A}, R_{2A}, R_{3A}, R_{6A}, R_{7A}, R_{8A} and R_{9A} is independently hydrogen or optionally substituted alkyl, aryl, alkylaryl, arylalkyl, heteroalkyl, heterocycle, alkylheterocycle, or heterocyclealkyl; and
each of R_{6B}, R_{7B}, R_{8B} and R_{9B} is independently hydrogen or alkyl optionally substituted with one or more hydroxy or halogen groups.

12. The method of claim 1, wherein the S1P promoting agent comprises a biologically active derivative of THI according to Formula VIII: 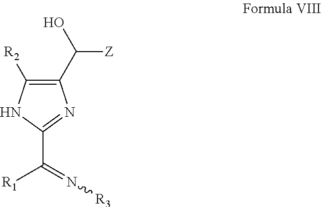
and pharmaceutically acceptable salts, esters, isomers and solvates thereof, wherein:
Z is optionally substituted alkyl; R₁ is OR_{1A}, NHOH, hydrogen, or optionally substituted alkyl, aryl, alkylaryl, arylalkyl, heteroalkyl, heterocycle, alkylheterocycle, or heterocyclealkyl;
R₂ is OR_{2A}, C(O)OR_{2A}, hydrogen, halogen, nitrile, or optionally substituted alkyl, aryl, alkylaryl, arylalkyl, heteroalkyl, heterocycle, alkylheterocycle, or heterocyclealkyl;
R₃ is OR_{3A}, N(R_{3A})₂, NHC(O)R_{3A}, NHSO₂R_{3A}, or hydrogen; and
each of R_{1A}, R_{2A}, and R_{3A} is independently hydrogen or optionally substituted alkyl, aryl, alkylaryl, arylalkyl, heteroalkyl, heterocycle, alkylheterocycle, or heterocyclealkyl.

13. The method of claim 1, wherein the S1P promoting agent comprises a biologically active derivative of THI according to Formula IX: 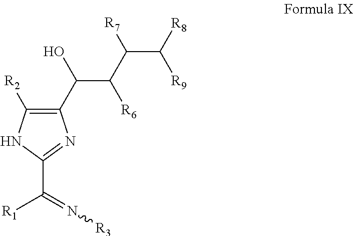
and pharmaceutically acceptable salts, esters, isomers and solvates thereof, wherein:
R₁ is OR_{1A}, NHOH, hydrogen, or optionally substituted alkyl, aryl, alkylaryl, arylalkyl, heteroalkyl, heterocycle, alkylheterocycle, or heterocyclealkyl;
R₃ is OR_{3A}, N(R_{3A})₂, NHC(O)R_{3A}, NHSO₂R_{3A}, or hydrogen;
R₆ is OR_{6A}, OC(O)R_{6A}, N(R_{6B})₂, NHC(O)R_{6B}, hydrogen, or halogen;
R₇ is OR_{7A}, OC(O)R_{7A}, N(R_{7B})₂, NHC(O)R_{7B}, hydrogen, or halogen;
R₈ is OR_{8A}, OC(O)R_{8A}, N(R_{8B})₂, NHC(O)R_{8B}, hydrogen, or halogen;
R₉ is CH₂OR_{9A}, CH₂OC(O)R_{9A}, N(R_{9B})₂, NHC(O)R_{9B}, hydrogen, or halogen; and
each of R_{1A}, R_{3A}, R_{6A}, R_{7A}, R_{8A} and R_{9A} is independently hydrogen or optionally substituted alkyl, aryl, alkylaryl, arylalkyl, heteroalkyl, heterocycle, alkylheterocycle, or heterocyclealkyl.

14. The method of claim 1, wherein the S1P promoting agent comprises a biologically active derivative of THI selected from:
(1R,2S,3R)-1-(2-(5-methylisoxazol-3-yl)-1H-imidazol-5-yl)butane-1,2,3,4-tetraol;
(1R,2S,3R)-1-(2-(5-ethylisoxazol-3-yl)-1H-imidazol-5-yl)butane-1,2,3,4-tetraol;
(1R,2S,3R)-1-(2-(isoxazol-3-yl)-1H-imidazol-5-yl)butane-1,2,3,4-tetraol;
(1R,2S,3R)-1-(2-(2-methylthiazol-4-yl)-1H-imidazol-4-yl)butane-1,2,3,4-tetraol;
(1R,2S,3R)-1-(2-(1-benzyl-1H-1,2,4-triazol-3-yl)-1H-imidazol-4-yl)butane-1,2,3,4-tetraol hydrochloride;
(1R,2S,3R)-1-(1H,1′H-2,2′-biimidazol-5-yl)butane-1,2,3,4-tetraol;
(1R,2S,3R)-1-(2-(5-methoxy-4,5-dihydroisoxazol-3-yl)-1H-imidazol-5-yl)butane-1,2,3,4-tetraol;
(1R,2S,3R)-1-(2-(isoxazol-3-yl)-1H-imidazol-4-yl)butane-1,2,3,4-tetraol;
(1R,2S,3R)-1-(2-(5-methyl-1H-pyrazol-3-yl)-1H-imidazol-5-yl)butane-1,2,3,4-tetraol; and
pharmaceutically acceptable salts, esters, isomers and solvates thereof.

15. The method of claim 1, wherein the S1P promoting agent comprises a biologically active derivative of THI selected from:
1-(4-((1R,2S,3R)-1,2,3,4-tetrahydroxybutyl)-1H-imidazol-2-yl)-ethanone oxime;
(E)-1-(4-((1R,2S,3R)-1,2,3,4-tetrahydroxybutyl)-1H-imidazol-2-yl)-ethanone oxime;
(Z)-1-(4-((1R,2S,3R)-1,2,3,4-tetrahydroxybutyl)-1H-imidazol-2-yl)-ethanone oxime;
1-(4-((1R,2S,3R)-1,2,3,4-tetrahydroxybutyl)-1H-imidazol-2-yl)ethanone O-methyl oxime;
(E)-1-(4-((1R,2S,3R)-1,2,3,4-tetrahydroxybutyl)-1H-imidazol-2-yl)ethanone O-methyl oxime;
(Z)-1-(4-((1R,2S,3R)-1,2,3,4-tetrahydroxybutyl)-1H-imidazol-2-yl)ethanone O-methyl oxime;
N′-(1-(4-((1R,2S,3R)-1,2,3,4-tetrahydroxybutyl)-1H-imidazol-2-yl)ethylidene)acetohydrazide;
4-methyl-N′-(1-(4-((1R,2S,3R)-1,2,3,4-tetrahydroxybutyl)-1H-imidazol-2-yl)ethylidene)benzenesulfonohydrazide;
(E)-4-methyl-N′-(1-(4-((1R,2S,3R)-1,2,3,4-tetrahydroxybutyl)-1H-imidazol-2-yl)ethylidene)benzenesulfonohydrazide;
(Z)-4-methyl-N′-(1-(4-((1R,2S,3R)-1,2,3,4-tetrahydroxybutyl)-1H-imidazol-2-yl)ethylidene)benzenesulfonohydrazide;
N′-(1-(4-((1R,2S,3R)-1,2,3,4-tetrahydroxybutyl)-1H-imidazol-2-yl)ethylidene)benzohydrazide;
ethyl 2-(1-(4-((1R,2S,3R)-1,2,3,4-tetrahydroxybutyl)-1H-imidazol-2-yl)ethylidene)hydrazinecarboxylate;
(E)-ethyl 2-(1-(4-((1R,2S,3R)-1,2,3,4-tetrahydroxybutyl)-1H-imidazol-2-yl)ethylidene)hydrazinecarboxylate;
(Z)-ethyl 2-(1-(4-((1R,2S,3R)-1,2,3,4-tetrahydroxybutyl)-1H-imidazol-2-yl)ethylidene)hydrazinecarboxylate;
N′-(1-(4-((1R,2S,3R)-1,2,3,4-tetrahydroxybutyl)-1H-imidazol-2-yl)ethylidene)nicotinohydrazide;
(E)-N′-(1-(4-((1R,2S,3R)-1,2,3,4-tetrahydroxybutyl)-1H-imidazol-2-yl)ethylidene)nicotinohydrazide;
(Z)—N′-(1-(4-((1R,2S,3R)-1,2,3,4-tetrahydroxybutyl)-1H-imidazol-2-yl)ethylidene)nicotinohydrazide;
3-chloro-N′-(1-(4-((1R,2S,3R)-1,2,3,4-tetrahydroxybutyl)-1H-imidazol-2-yl)ethylidene)benzohydrazide;
4-fluoro-N′-(1-(4-((1R,2S,3R)-1,2,3,4-tetrahydroxybutyl)-1H-imidazol-2-yl)ethylidene)benzohydrazide;
(E)-4-fluoro-N′-(1-(4-((1R,2S,3R)-1,2,3,4-tetrahydroxybutyl)-1H-imidazol-2-yl)ethylidene)benzohydrazide;
(Z)-4-fluoro-N′-(1-(4-((1R,2S,3R)-1,2,3,4-tetrahydroxybutyl)-1H-imidazol-2-yl)ethylidene)benzohydrazide;
6-amino-N′-(1-(4-((1R,2S,3R)-1,2,3,4-tetrahydroxybutyl)-1H-imidazol-2-yl)ethylidene)nicotinohydrazide;
(E)-6-amino-N′-(1-(4-((1R,2S,3R)-1,2,3,4-tetrahydroxybutyl)-1H-imidazol-2-yl)ethylidene)nicotinohydrazide;
(Z)-6-amino-N′-(1-(4-((1R,2S,3R)-1,2,3,4-tetrahydroxybutyl)-1H-imidazol-2-yl)ethylidene)nicotinohydrazide;
N′-(1-(4-((1R,2S,3R)-1,2,3,4-tetrahydroxybutyl)-1H-imidazol-2-yl)ethylidene)isonicotinohydrazide;
(E)-N′-(1-(4-((1R,2S,3R)-1,2,3,4-tetrahydroxybutyl)-1H-imidazol-2-yl)ethylidene)isonicotinohydrazide;
(Z)—N′-(1-(4-((1R,2S,3R)-1,2,3,4-tetrahydroxybutyl)-1H-imidazol-2-yl)ethylidene)isonicotinohydrazide;
N′-(1-(4-((1R,2S,3R)-1,2,3,4-tetrahydroxybutyl)-1H-imidazol-2-yl)ethylidene)biphenyl-3-carbohydrazide;
(E)-N′-(1-(4-((1R,2S,3R)-1,2,3,4-tetrahydroxybutyl)-1H-imidazol-2-yl)ethylidene)biphenyl-3-carbohydrazide;
(Z)—N′-(1-(4-((1R,2S,3R)-1,2,3,4-tetrahydroxybutyl)-1H-imidazol-2-yl)ethylidene)biphenyl-3-carbohydrazide; and
pharmaceutically acceptable salts, esters, isomers and solvates thereof.

16. The method of claim 1, wherein administration of the therapeutically effective amount of the S1P promoting composition in the subject suffering from the degenerative muscle condition increases the presence of S1P in muscle tissue of the subject.

17. The method of claim 1, wherein administration of the therapeutically effective amount of the S1P promoting composition in the subject suffering from the degenerative muscle condition promotes proliferation of satellite cells in muscle tissue of the subject.

18. The method of claim 1, wherein administration of the therapeutically effective amount of the S1P promoting composition in the subject suffering from the degenerative muscle condition promotes an increase in muscle fiber size in muscle tissue of the subject.

19. The method of claim 1, wherein administration of the therapeutically effective amount of the S1P promoting composition in the subject suffering from the degenerative muscle condition inhibits fibrosis in muscle tissue of the subject.

20. The method of claim 1, wherein administration of the therapeutically effective amount of the S1P promoting composition in the subject suffering from the degenerative muscle condition inhibits fat deposition in muscle tissue of the subject.

21. A method of inhibiting fibrosis in muscle tissue in a subject suffering from a degenerative muscle condition, the method comprising administering a therapeutically effective amount of an S1P promoting composition comprising one or more S1P promoting agent selected from at least one of S1P, THI, a biologically active derivative of THI, and pharmaceutically acceptable salts, esters, isomers and solvates thereof.

22. A method of promoting proliferation of satellite cells in muscle tissue of a subject suffering from a degenerative muscle condition, the method comprising administering a therapeutically effective amount of an S1P promoting composition comprising one or more S1P promoting agent selected from at least one of S1P, THI, a biologically active derivative of THI, and pharmaceutically acceptable salts, esters, isomers and solvates thereof.

23. A method of promoting an increase in muscle fiber size or number in muscle tissue of a subject suffering from a degenerative muscle condition, the method comprising administering a therapeutically effective amount of an S1P promoting composition comprising one or more S1P promoting agent selected from at least one of S1P, THI, a biologically active derivative of THI, and pharmaceutically acceptable salts, esters, isomers and solvates thereof.

24. A method of inhibiting fat deposition in muscle tissue of a subject suffering from a degenerative muscle condition, the method comprising administering a therapeutically effective amount of an S1P promoting composition comprising one or more S1P promoting agent selected from at least one of S1P, THI, a biologically active derivative of THI, and pharmaceutically acceptable salts, esters, isomers and solvates thereof.

25. A method of promoting muscle regeneration in muscle tissue of a subject suffering from a degenerative muscle condition, the method comprising administering a therapeutically effective amount of an S1P promoting composition comprising one or more S1P promoting agent selected from at least one of S1P, THI, a biologically active derivative of THI, a sphingosine analog, and pharmaceutically acceptable salts, esters, isomers and solvates thereof.

26. The method of claim 1, wherein the degenerative muscle condition is selected from muscle injury, sarcopenia and muscular dystrophy.

27. The method of claim 1, wherein the degenerative muscle condition is selected from at least one of Duchenne Muscular Dystrophy, Becker Muscular Dystrophy, muscle injury resulting in loss of muscle tissue, muscle atrophy, or muscle wasting, muscle overuse, muscle disuse atrophy, dysferlinopathy, denervation muscle atrophy, AIDS/HIV, diabetes, chronic obstructive pulmonary disease, kidney disease, cancer, aging, autoimmune disease, polymyositis, and dermatomyositis.

28. A pharmaceutical composition for the treatment of a degenerative muscle condition in a subject, the pharmaceutical composition comprising a therapeutically effective amount of a S1P promoting composition comprising an S1P promoting agent selected from S1P, THI, a biologically active derivative of THI, a sphingosine analog and pharmaceutically acceptable salts, esters, isomers and solvates thereof.

29. The pharmaceutical composition of claim 28, wherein the S1P promoting agent is S1P.

30. A pharmaceutical composition according to claim 28, wherein the S1P promoting agent is a biologically active derivative of THI according to Formula III: 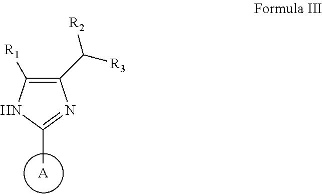
and pharmaceutically acceptable salts, esters, isomers and solvates thereof, wherein:
A is an optionally substituted heterocycle;
R₁ is OR_{1A}, OC(O)R_{1A}, C(O)OR_{1A}, hydrogen, halogen, nitrile, or optionally substituted alkyl, aryl, alkylaryl, arylalkyl, heteroalkyl, heterocycle, alkylheterocycle, or heterocyclealkyl;
R₂ is OR_{2A}, OC(O)R_{2A}, hydrogen, halogen, or optionally substituted alkyl, aryl, alkylaryl, arylalkyl, heteroalkyl, heterocycle, alkylheterocycle, or heterocyclealkyl;
R₃ is N(R_{3A})₂, hydrogen, hydroxy, or optionally substituted alkyl, aryl, alkylaryl, arylalkyl, heteroalkyl, heterocycle, alkylheterocycle, or heterocyclealkyl;
and each of R_{1A}, R_{2A}, and R_{3A} is independently hydrogen or optionally substituted alkyl, aryl, alkylaryl, arylalkyl, heteroalkyl, heterocycle, alkylheterocycle, or heterocyclealkyl.

31. A pharmaceutical composition according to claim 28, wherein the S1P promoting agent is a biologically active derivative of THI according to Formula IV: 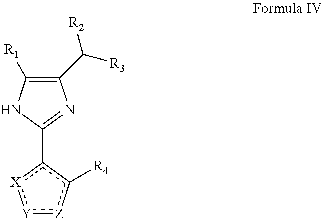
and pharmaceutically acceptable salts, esters, isomers and solvates thereof, wherein:
X is CR₄, CHR₄, N, NR₉, O or S; Y is CR₄, CHR₄, N, NR₉, O or S; Z is CR₄, CHR₄, N, NR₉, O or S;
R₁ is OR_{1A}, C(O)OR_{1A}, hydrogen, halogen, nitrile, or optionally substituted alkyl, aryl, alkylaryl, arylalkyl, heteroalkyl, heterocycle, alkylheterocycle, or heterocyclealkyl;
R₂ is OR_{2A}, OC(O)R_{2A}, hydrogen, halogen, or optionally substituted alkyl, aryl, alkylaryl, arylalkyl, heteroalkyl, heterocycle, alkylheterocycle, or heterocyclealkyl;
R₃ is N(R_{3A})₂, hydrogen, hydroxy, or optionally substituted alkyl, aryl, alkylaryl, arylalkyl, heteroalkyl, heterocycle, alkylheterocycle, or heterocyclealkyl;
R₄ is OR_{4A}, OC(O)R_{4A}, hydrogen, halogen, or optionally substituted alkyl, aryl, alkylaryl, arylalkyl, heteroalkyl, heterocycle, alkylheterocycle, or heterocyclealkyl;
each R₉ is independently hydrogen or optionally substituted alkyl, aryl, alkylaryl, arylalkyl, heteroalkyl, heterocycle, alkylheterocycle, or heterocyclealkyl; and
each of R_{1A}, R_{2A}, R_{3A} and R_{4A} is independently hydrogen or optionally substituted alkyl, aryl, alkylaryl, arylalkyl, heteroalkyl, heterocycle, alkylheterocycle, or heterocyclealkyl.

32. A pharmaceutical composition according to claim 28, wherein the S1P promoting agent is a biologically active derivative of THI according to Formula V: 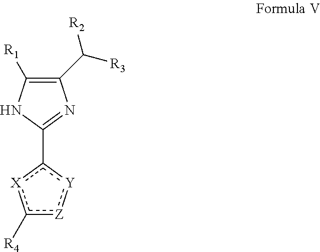
and pharmaceutically acceptable salts, esters, isomers and solvates thereof, wherein:
X is CR₄, CHR₄, N, NR₉, O or S;
Y is CR₄, CHR₄, N, NR₉, O or S;
Z is CR₄, CHR₄, N, NR₉, O or S;
R₁ is OR_{1A}, C(O)OR_{1A}, hydrogen, halogen, nitrile, or optionally substituted alkyl, aryl, alkylaryl, arylalkyl, heteroalkyl, heterocycle, alkylheterocycle, or heterocyclealkyl;
R₂ is OR_{2A}, OC(O)R_{2A}, hydrogen, halogen, or optionally substituted alkyl, aryl, alkylaryl, arylalkyl, heteroalkyl, heterocycle, alkylheterocycle, or heterocyclealkyl;
R₃ is N(R_{3A})₂, hydrogen, hydroxy, or optionally substituted alkyl, aryl, alkylaryl, arylalkyl, heteroalkyl, heterocycle, alkylheterocycle, or heterocyclealkyl;
R₄ is independently OR_{4A}, OC(O)R_{4A}, hydrogen, halogen, or optionally substituted alkyl, aryl, alkylaryl, arylalkyl, heteroalkyl, heterocycle, alkylheterocycle, or heterocyclealkyl;
each R₉ is independently hydrogen or optionally substituted alkyl, aryl, alkylaryl, arylalkyl, heteroalkyl, heterocycle, alkylheterocycle, or heterocyclealkyl;
and each of R_{1A}, R_{2A}, R_{3A} and R_{4A} is independently hydrogen or optionally substituted alkyl, aryl, alkylaryl, arylalkyl, heteroalkyl, heterocycle, alkylheterocycle, or heterocyclealkyl.

33. A pharmaceutical composition according to claim 28, wherein the S1P promoting agent is an SPL inhibitor comprising a biologically active derivative of THI according to Formula VI: 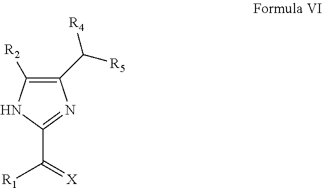
and pharmaceutically acceptable salts, esters, isomers and solvates thereof, wherein:
X is O or NR₃;
R₁ is OR_{1A}, NHOH, hydrogen, or optionally substituted alkyl, aryl, alkylaryl, arylalkyl, heteroalkyl, heterocycle, alkylheterocycle, or heterocyclealkyl;
R₂ is OR_{2A}, C(O)OR_{2A}, hydrogen, halogen, nitrile, or optionally substituted alkyl, aryl, alkylaryl, arylalkyl, heteroalkyl, heterocycle, alkylheterocycle, or heterocyclealkyl;
R₃ is OR_{3A}, N(R_{3A})₂, NHC(O)R_{3A}, NHSO₂R_{3A}, or hydrogen;
R₄ is OR_{4A}, OC(O)R_{4A}, hydrogen, halogen, or optionally substituted alkyl, aryl, alkylaryl, arylalkyl, heteroalkyl, heterocycle, alkylheterocycle, or heterocyclealkyl;
R₅ is N(R_{5A})₂, hydrogen, hydroxy, or optionally substituted alkyl, aryl, alkylaryl, arylalkyl, heteroalkyl, heterocycle, alkylheterocycle, or heterocyclealkyl; and
each of R_{1A}, R_{2A}, R_{3A}, R_{4A}, and R_{5A} is independently hydrogen or optionally substituted alkyl, aryl, alkylaryl, arylalkyl, heteroalkyl, heterocycle, alkylheterocycle, or heterocyclealkyl.

34. A pharmaceutical composition according to claim 28, wherein the S1P promoting agent is a biologically active derivative of THI according to Formula VII: 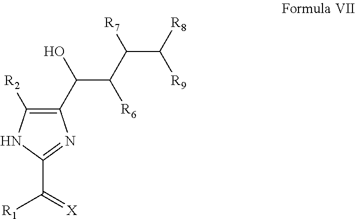
and pharmaceutically acceptable salts, esters, isomers and solvates thereof, wherein:
X is O or NR₃; R₁ is OR_{1A}, NHOH, hydrogen, or optionally substituted alkyl, aryl, alkylaryl, arylalkyl, heteroalkyl, heterocycle, alkylheterocycle, or heterocyclealkyl;
R₂ is OR_{2A}, C(O)OR_{2A}, hydrogen, halogen, nitrile, or optionally substituted alkyl, aryl, alkylaryl, arylalkyl, heteroalkyl, heterocycle, alkylheterocycle, or heterocyclealkyl;
R₃ is OR_{3A}, N(R_{3A})₂, NHC(O)R_{3A}, NHSO₂R_{3A}, or hydrogen;
R₆ is OR_{6A}, OC(O)R_{6A}, N(R_{6B})₂, NHC(O)R_{6B}, hydrogen, or halogen;
R₇ is OR_{7A}, OC(O)R_{7A}, N(R_{7B})₂, NHC(O)R_{7B}, hydrogen, or halogen;
R₈ is OR_{8A}, OC(O)R_{8A}, N(R_{8B})₂, NHC(O)R_{8B}, hydrogen, or halogen;
R₉ is CH₂OR_{9A}, CH₂OC(O)R_{9A}, N(R_{9B})₂, NHC(O)R_{9B}, hydrogen, or halogen;
each of R_{1A}, R_{2A}, R_{3A}, R_{6A}, R_{7A}, R_{8A} and R_{9A} is independently hydrogen or optionally substituted alkyl, aryl, alkylaryl, arylalkyl, heteroalkyl, heterocycle, alkylheterocycle, or heterocyclealkyl;
and each of R_{6B}, R_{7B}, R_{8B} and R_{9B} is independently hydrogen or alkyl optionally substituted with one or more hydroxy or halogen groups.

35. A pharmaceutical composition according to claim 28, wherein the S1P promoting agent is a biologically active derivative of THI according to Formula VIII: 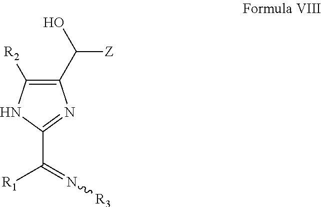
and pharmaceutically acceptable salts, esters, isomers and solvates thereof, wherein:
Z is optionally substituted alkyl; R₁ is OR_{1A}, NHOH, hydrogen, or optionally substituted alkyl, aryl, alkylaryl, arylalkyl, heteroalkyl, heterocycle, alkylheterocycle, or heterocyclealkyl;
R₂ is OR_{2A}, C(O)OR_{2A}, hydrogen, halogen, nitrile, or optionally substituted alkyl, aryl, alkylaryl, arylalkyl, heteroalkyl, heterocycle, alkylheterocycle, or heterocyclealkyl;
R₃ is OR_{3A}, N(R_{3A})₂, NHC(O)R_{3A}, NHSO₂R_{3A}, or hydrogen; and
each of R_{1A}, R_{2A}, and R_{3A} is independently hydrogen or optionally substituted alkyl, aryl, alkylaryl, arylalkyl, heteroalkyl, heterocycle, alkylheterocycle, or heterocyclealkyl.

36. A pharmaceutical composition according to claim 28, wherein the S1P promoting agent is a biologically active derivative of THI according to Formula IX: 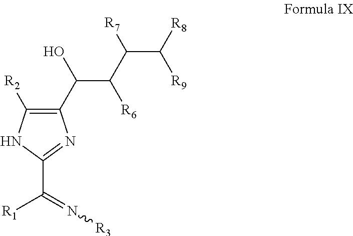
and pharmaceutically acceptable salts, esters, isomers and solvates thereof, wherein:
R₁ is OR_{1A}, NHOH, hydrogen, or optionally substituted alkyl, aryl, alkylaryl, arylalkyl, heteroalkyl, heterocycle, alkylheterocycle, or heterocyclealkyl;
R₃ is OR_{3A}, N(R_{3A})₂, NHC(O)R_{3A}, NHSO₂R_{3A}, or hydrogen;
R₆ is OR_{6A}, OC(O)R_{6A}, N(R_{6B})₂, NHC(O)R_{6B}, hydrogen, or halogen;
R₇ is OR_{7A}, OC(O)R_{7A}, N(R_{7B})₂, NHC(O)R_{7B}, hydrogen, or halogen;
R₈ is OR_{8A}, OC(O)R_{8A}, N(R_{8B})₂, NHC(O)R_{8B}, hydrogen, or halogen;
R₉ is CH₂OR_{9A}, CH₂OC(O)R_{9A}, N(R_{9B})₂, NHC(O)R_{9B}, hydrogen, or halogen; and
each of R_{1A}, R_{3A}, R_{6A}, R_{7A}, R_{8A} and R_{9A} is independently hydrogen or optionally substituted alkyl, aryl, alkylaryl, arylalkyl, heteroalkyl, heterocycle, alkylheterocycle, or heterocyclealkyl.

37. A pharmaceutical composition according to claim 28, wherein the S1P promoting agent is a biologically active derivative of THI selected from:
(1R,2S,3R)-1-(2-(5-methylisoxazol-3-yl)-1H-imidazol-5-yl)butane-1,2,3,4-tetraol;
(1R,2S,3R)-1-(2-(5-ethylisoxazol-3-yl)-1H-imidazol-5-yl)butane-1,2,3,4-tetraol;
(1R,2S,3R)-1-(2-(isoxazol-3-yl)-1H-imidazol-5-yl)butane-1,2,3,4-tetraol;
(1R,2S,3R)-1-(2-(2-methylthiazol-4-yl)-1H-imidazol-4-yl)butane-1,2,3,4-tetraol;
(1R,2S,3R)-1-(2-(1-benzyl-1H-1,2,4-triazol-3-yl)-1H-imidazol-4-yl)butane-1,2,3,4-tetraol hydrochloride;
(1R,2S,3R)-1-(1H,1′H-2,2′-biimidazol-5-yl)butane-1,2,3,4-tetraol;
(1R,2S,3R)-1-(2-(5-methoxy-4,5-dihydroisoxazol-3-yl)-1H-imidazol-5-yl)butane-1,2,3,4-tetraol;
(1R,2S,3R)-1-(2-(isoxazol-3-yl)-1H-imidazol-4-yl)butane-1,2,3,4-tetraol (1R,2S,3R)-1-(2-(5-methyl-1H-pyrazol-3-yl)-1H-imidazol-5-yl)butane-1,2,3,4-tetraol; and
pharmaceutically acceptable salts, esters, isomers and solvates thereof.

38. A pharmaceutical composition according to claim 28, wherein the S1P promoting agent is a biologically active derivative of THI selected from:
1-(4-((1R,2S,3R)-1,2,3,4-tetrahydroxybutyl)-1H-imidazol-2-yl)-ethanone oxime;
(E)-1-(4-((1R,2S,3R)-1,2,3,4-tetrahydroxybutyl)-1H-imidazol-2-yl)-ethanone oxime;
(Z)-1-(4-((1R,2S,3R)-1,2,3,4-tetrahydroxybutyl)-1H-imidazol-2-yl)-ethanone oxime;
1-(4-((1R,2S,3R)-1,2,3,4-tetrahydroxybutyl)-1H-imidazol-2-yl)ethanone O-methyl oxime;
(E)-1-(4-((1R,2S,3R)-1,2,3,4-tetrahydroxybutyl)-1H-imidazol-2-yl)ethanone O-methyl oxime;
(Z)-1-(4-((1R,2S,3R)-1,2,3,4-tetrahydroxybutyl)-1H-imidazol-2-yl)ethanone O-methyl oxime;
N′-(1-(4-((1R,2S,3R)-1,2,3,4-tetrahydroxybutyl)-1H-imidazol-2-yl)ethylidene)acetohydrazide;
4-methyl-N′-(1-(4-((1R,2S,3R)-1,2,3,4-tetrahydroxybutyl)-1H-imidazol-2-yl)ethylidene)benzenesulfonohydrazide;
(E)-4-methyl-N′-(1-(4-((1R,2S,3R)-1,2,3,4-tetrahydroxybutyl)-1H-imidazol-2-yl)ethylidene)benzenesulfonohydrazide;
(Z)-4-methyl-N′-(1-(4-((1R,2S,3R)-1,2,3,4-tetrahydroxybutyl)-1H-imidazol-2-yl)ethylidene)benzenesulfonohydrazide;
N′-(1-(4-((1R,2S,3R)-1,2,3,4-tetrahydroxybutyl)-1H-imidazol-2-yl)ethylidene)benzohydrazide;
ethyl 2-(1-(4-((1R,2S,3R)-1,2,3,4-tetrahydroxybutyl)-1H-imidazol-2-yl)ethylidene)hydrazinecarboxylate;
(E)-ethyl 2-(1-(4-((1R,2S,3R)-1,2,3,4-tetrahydroxybutyl)-1H-imidazol-2-yl)ethylidene)hydrazinecarboxylate;
(Z)-ethyl 2-(1-(4-((1R,2S,3R)-1,2,3,4-tetrahydroxybutyl)-1H-imidazol-2-yl)ethylidene)hydrazinecarboxylate;
N′-(1-(4-((1R,2S,3R)-1,2,3,4-tetrahydroxybutyl)-1H-imidazol-2-yl)ethylidene)nicotinohydrazide;
(E)-N′-(1-(4-((1R,2S,3R)-1,2,3,4-tetrahydroxybutyl)-1H-imidazol-2-yl)ethylidene)nicotinohydrazide;
(Z)—N′-(1-(4-((1R,2S,3R)-1,2,3,4-tetrahydroxybutyl)-1H-imidazol-2-yl)ethylidene)nicotinohydrazide;
3-chloro-N′-(1-(4-((1R,2S,3R)-1,2,3,4-tetrahydroxybutyl)-1H-imidazol-2-yl)ethylidene)benzohydrazide;
4-fluoro-N′-(1-(4-((1R,2S,3R)-1,2,3,4-tetrahydroxybutyl)-1H-imidazol-2-yl)ethylidene)benzohydrazide;
(E)-4-fluoro-N′-(1-(4-((1R,2S,3R)-1,2,3,4-tetrahydroxybutyl)-1H-imidazol-2-yl)ethylidene)benzohydrazide;
(Z)-4-fluoro-N′-(1-(4-((1R,2S,3R)-1,2,3,4-tetrahydroxybutyl)-1H-imidazol-2-yl)ethylidene)benzohydrazide;
6-amino-N′-(1-(4-((1R,2S,3R)-1,2,3,4-tetrahydroxybutyl)-1H-imidazol-2-yl)ethylidene)nicotinohydrazide;
(E)-6-amino-N′(1-(4-((1R,2S,3R)-1,2,3,4-tetrahydroxybutyl)-1H-imidazol-2-yl)ethylidene)nicotinohydrazide;
(Z)-6-amino-N′-(1-(4-((1R,2S,3R)-1,2,3,4-tetrahydroxybutyl)-1H-imidazol-2-yl)ethylidene)nicotinohydrazide;
N′-(1-(4-((1R,2S,3R)-1,2,3,4-tetrahydroxybutyl)-1H-imidazol-2-yl)ethylidene)isonicotinohydrazide;
(E)-N′-(1-(4-((1R,2S,3R)-1,2,3,4-tetrahydroxybutyl)-1H-imidazol-2-yl)ethylidene)isonicotinohydrazide;
(Z)—N′-(1-(4-((1R,2S,3R)-1,2,3,4-tetrahydroxybutyl)-1H-imidazol-2-yl)ethylidene)isonicotinohydrazide;
N′-(1-(4-((1R,2S,3R)-1,2,3,4-tetrahydroxybutyl)-1H-imidazol-2-yl)ethylidene)biphenyl-3-carbohydrazide;
(E)-N′-(1-(4-((1R,2S,3R)-1,2,3,4-tetrahydroxybutyl)-1H-imidazol-2-yl)ethylidene)biphenyl-3-carbohydrazide;
(Z)—N′-(1-(4-((1R,2S,3R)-1,2,3,4-tetrahydroxybutyl)-1H-imidazol-2-yl)ethylidene)biphenyl-3-carbohydrazide; and
pharmaceutically acceptable salts, esters, isomers and solvates thereof.

39. A pharmaceutical composition according to claim 28, wherein the S1P promoting agent is a sphingosine analog according to Formula X: 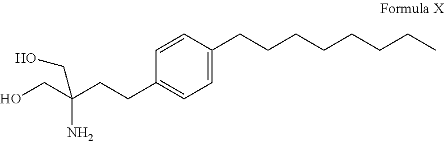 [SMILES: CCCCCCCCc1ccc(CCC(N)(CO)CO)cc1]
and pharmaceutically acceptable salts, esters, isomers and solvates thereof.

40. A pharmaceutical composition according to claim 28, wherein the degenerative muscle condition is sarcopenia and the therapeutically effective amount of the S1P promoting agent included in the S1P promoting composition is sufficient to promote proliferation of satellite cells in muscle tissue of a subject to which the pharmaceutical composition is administered.

41. A pharmaceutical composition according to claim 28, wherein the degenerative muscle condition is sarcopenia and the therapeutically effective amount of the S1P promoting agent included in the S1P promoting composition is sufficient to inhibit fat deposition in muscle tissue of a subject to which the pharmaceutical composition is administered.

42. A pharmaceutical composition according to claim 28, wherein the degenerative muscle condition is sarcopenia and the therapeutically effective amount of the S1P promoting agent included in the S1P promoting composition is sufficient to inhibit fibrosis in muscle tissue of a subject to which the pharmaceutical composition is administered.

43. A pharmaceutical composition according to claim 28, wherein the degenerative muscle condition is sarcopenia and the therapeutically effective amount of the S1P promoting agent included in the S1P promoting composition is sufficient to promote an increase in the size of muscle fiber in muscle tissue of a subject to which the pharmaceutical composition is administered.

44. A pharmaceutical composition according to claim 28, wherein the degenerative muscle condition is sarcopenia and the therapeutically effective amount of the S1P promoting agent included in the S1P promoting composition is sufficient to promote regeneration of muscle tissue of a subject to which the pharmaceutical composition is administered.

45. A pharmaceutical composition according to claim 28, wherein the degenerative muscle condition is muscular dystrophy and the therapeutically effective amount of the S1P promoting agent included in the S1P promoting composition is sufficient to promote proliferation of satellite cells in muscle tissue of a subject to which the pharmaceutical composition is administered.

46. A pharmaceutical composition according to claim 28, wherein the degenerative muscle condition is muscular dystrophy and the therapeutically effective amount of the S1P promoting agent included in the S1P promoting composition is sufficient to inhibit fat deposition in muscle tissue of a subject to which the pharmaceutical composition is administered.

47. A pharmaceutical composition according to claim 28, wherein the degenerative muscle condition is muscular dystrophy and the therapeutically effective amount of the S1P promoting agent included in the S1P promoting composition is sufficient to inhibit fibrosis in muscle tissue of a subject to which the pharmaceutical composition is administered.

48. A pharmaceutical composition according to claim 28, wherein the degenerative muscle condition is muscular dystrophy and the therapeutically effective amount of the S1P promoting agent included in the S1P promoting composition is sufficient to promote an increase in size of muscle fiber in muscle tissue of a subject to which the pharmaceutical composition is administered.

49. A pharmaceutical composition according to claim 28, wherein the degenerative muscle condition is muscular dystrophy and the therapeutically effective amount of the S1P promoting agent included in the S1P promoting composition is sufficient to promote regeneration of muscle tissue of a subject to which the pharmaceutical composition is administered.

50. A pharmaceutical composition according to claim 28, wherein the degenerative muscle condition is Duchenne Muscular Dystrophy or Becker Muscular Dystrophy.

51. A pharmaceutical composition according to claim 28, wherein the degenerative muscle condition is associated with at least one of muscle disuse atrophy, denervation muscle atrophy, dysferlinopathy, AIDS/HIV, diabetes, chronic obstructive pulmonary disease, kidney disease, cancer, aging, autoimmune disease, polymyositis, and dermatomyositis.

52. A pharmaceutical composition according to claim 28, wherein the degenerative muscle condition is a muscle injury and the therapeutically effective amount of the S1P promoting agent included in the S1P promoting composition is sufficient to promote proliferation of satellite cells in muscle tissue of a subject to which the pharmaceutical composition is administered.

53. A pharmaceutical composition according to claim 28, wherein the degenerative muscle condition is a muscle injury and the therapeutically effective amount of the S1P promoting agent included in the S1P promoting composition is sufficient to inhibit fat deposition in muscle tissue of a subject to which the pharmaceutical composition is administered.

54. A pharmaceutical composition according to claim 28, wherein the degenerative muscle condition is a muscle injury and the therapeutically effective amount of the S1P promoting agent included in the S1P promoting composition is sufficient to inhibit fibrosis in muscle tissue of a subject to which the pharmaceutical composition is administered.

55. A pharmaceutical composition according to claim 28, wherein the degenerative muscle condition is a muscle injury and the therapeutically effective amount of the S1P promoting agent included in the S1P promoting composition is sufficient to promote an increase in size of muscle fiber in muscle tissue of a subject to which the pharmaceutical composition is administered.

56. A pharmaceutical composition according to claim 28, wherein the degenerative muscle condition is a muscle injury and the therapeutically effective amount of the S1P promoting agent included in the S1P promoting composition is sufficient to promote regeneration of muscle tissue of a subject to which the pharmaceutical composition is administered.

57. A pharmaceutical composition according to claim 52, wherein the muscle injury is selected from a muscle injury, including an acute muscle injury, resulting in loss of muscle tissue, muscle atrophy or muscle wasting, and muscle overuse.

58. A method of treating a degenerative muscle condition in a subject, the method comprising administering to the subject a therapeutically effective amount of THI and pharmaceutically acceptable salts, esters, isomers and solvates thereof.

59. A method according to claim 58, wherein the degenerative muscle condition is selected from sarcopenia and muscular dystrophy.

60. A method according to claim 58, wherein the degenerative muscle condition is selected from at least one of Duchenne Muscular Dystrophy, Becker Muscular Dystrophy, muscle injury, muscle disuse atrophy, denervation muscle atrophy, dysferlinopathy, AIDS/HIV, diabetes, chronic obstructive pulmonary disease, kidney disease, cancer, aging, autoimmune disease, polymyositis, and dermatomyositis.

61. A method according to claim 60, wherein the degenerative muscle condition is a muscle injury and the muscle injury is selected from a muscle injury, including an acute muscle injury, resulting in loss of muscle tissue, muscle atrophy or muscle wasting, and muscle overuse.

62. A method according to claim 58, wherein administration of the therapeutically effective amount of THI increases the presence of S1P in muscle tissue of the subject.

63. A method according to claim 58, wherein administration of the therapeutically effective amount of THI promotes proliferation of satellite cells in muscle tissue of the subject.

64. A method according to claim 58, wherein administration of the therapeutically effective amount of THI promotes an increase in muscle fiber size in muscle tissue of the subject.

65. A method according to claim 58, wherein administration of the therapeutically effective amount of THI inhibits fibrosis in muscle tissue of the subject.

66. A method according to claim 58, wherein administration of the therapeutically effective amount of THI inhibits fat deposition in muscle tissue of the subject.
